(19) United States
(12) Patent Application Publication
(10) Pub. No.: US 2024/0018151 A1
(43) Pub. Date: Jan. 18, 2024

(54) NOVEL INHIBITORS OF PIKFYVE AND METHODS USING SAME

(71) Applicant: TME Therapeutics LLC, Acton, MA (US)

(72) Inventor: Vinod F. PATEL, Acton, MA (US)

(21) Appl. No.: 18/249,728

(22) PCT Filed: Oct. 19, 2021
(86) PCT No.: PCT/US2021/055651

§ 371 Date: Apr. 19, 2023

Related U.S. Application Data
(60) Provisional application No. 63/093,705, filed on Oct. 19, 2020.
(60) Provisional application No. 63/253,404, filed on Oct. 7, 2021.

(51) Int. Cl. C07D 487/04; A61P 35/00; A61P 31/16

(52) U.S. Cl. CPC C07D 487/04; A61P 35/00; A61P 31/16

(57) ABSTRACT

The invention relates to novel inhibitors of the PIKFYVE, a phosphoinositide kinase, useful for the treatment of diseases or disorders characterized by dysregulation of phosphoinositide mediated signal transduction pathways, including hyperproliferative diseases, autoimmune diseases, Crohn's disease, psoriasis, neurological diseases, diabetes, corneal fleck dystrophy, and viral infection (including HIV, Ebola, and coronavirus infections). The invention further relates to pharmaceutical compositions comprising PIKFYVE inhibitors and methods of treatment of such diseases and disorders.

### CROSS-REFERENCE TO RELATED APPLICATIONS

This application is an international application which claims priority to, and the benefit of, U.S. Provisional Application Ser. No. 63/093,705, filed on Oct. 19, 2020, and U.S. Provisional Application Ser. No. 63/253,404, filed on Oct. 7, 2021, the contents of each of which are hereby incorporated by reference in their entireties.

### FIELD OF THE INVENTION

The invention relates to novel inhibitors of the PIKFYVE, a phosphoinositide kinase, useful for the treatment of diseases or disorders characterized by dysregulation of phosphoinositide-mediated signal transduction pathways, including hyperproliferative diseases (such as MET or RAS dependent cancers, including prostate cancer), autoimmune diseases, Crohn's disease, psoriasis, neurological diseases, diabetes, corneal fleck dystrophy, and viral infection (including HIV, Ebola, and coronavirus infections). The invention further relates to pharmaceutical compositions comprising PIKFYVE inhibitors and methods of treatment of such diseases and disorders.

### BACKGROUND OF THE INVENTION

Protein kinases represent a large family of proteins which play a variety of crucial roles in the regulation of a wide range of cellular processes. Such kinases include lipid kinases, serine-threonine protein kinases, tyrosine protein kinases, and other kinases. Inhibition of various protein kinases, especially selective inhibition, has become an important strategy in treating many diseases and disorders.

PIKFYVE (or PIKfyve) is a phosphoinositide kinase whose primary function is the phosphorylation of phosphoinositide-3-phosphate (PtdIns3P or PI3P) to form phosphoinositide-3,5-diphosphate (PtdIns(3,5)P2 or PI(3,5P)2). PIKFYVE also phosphorylates phosphoinositide to form phosphoinositide-5-phosphate (PtdIns5P or PI5P). PIKFYVE includes an FYVE-finger domain, a zinc-finger domain, which is responsible for binding of the protein to PI3P. PI3P is a membrane bound lipid, and binding of PIKFYVE to PI3P can result in insertion of the kinase into cellular membranes, such as endosomes, vacuoles and other intracellular vesicles.

PI(3,5)P2 is one of seven phosphoinositides found in eukaryotic cell membranes, along with the more abundant PI3P, PI4P (phosphoinositide-4-phosphate), PI5P, PI(4,5)P2 (phosphoinositide-4,5-diphosphate), and PIP3 (phosphoinositide-3,4,5-triphosphate). Phosphoinositides are membrane-bound regulatory lipids, and they participate in signaling events that control cytoskeletal dynamics, intracellular membrane trafficking, cell proliferation, and many other cellular functions. Like other phosphoinositides, PI(3,5)P2 acts as a signaling molecule in various cellular signaling pathways, as well as being a precursor for the synthesis of PI5P.

PI(3,5)P2 is present at the lowest concentration of the phosphoinositides and after formation is it is rapidly dephosphorylated back to PI3P by the phosphatase Sac3. PIKFYVE is the only kinase which forms PI(3,5)P2 and unusually, PIKFYVE exists in a large multi-protein complex, the PAS complex, also comprising Sac3. The presence of a kinase and a phosphatase with opposite activities in the same complex suggests the critical importance of the concentration of PI(3,5)P2 to normal cell functioning. In addition to PIKFYVE and Sac1, the PAS complex also contains ArPIKFYVE, a regulatory protein which scaffolds the complex. Studies show that in most eukaryotic organisms, silencing or knockout of PIKFYVE function (or its equivalent) is lethal during embryonic development, further suggesting the critical importance of this protein and its product PI(3,5)P2.

PI(3,5)P2 helps regulate endosomal operations, such as membrane fission and fusion, that maintain endosomal homeostasis and support trafficking pathways throughout cells. Inhibition of PIKFYVE function in in-vitro cell studies shows the formation of numerous cytosolic vacuoles which grow larger over time, but such defects are shown to be reversible upon resupply of PI(3,5)P2 or functioning PIKFYVE. While homozygous knockout models of PIKFYVE are lethal, heterozygous knockout it not. This as well as other studies suggest that PIKFYVE activity, and consequently PI(3,5)P2 cellular concentration, is normally in excess of that required for normal cell functioning.

Under the sustained activation of glutamate receptors, PIKFYVE has also been shown to facilitate the lysosomal degradation of type 1.2 voltage-dependent calcium channels in neurons. This helps protect neurons from excitotoxicity, and suggests a role in treating or preventing central nervous system dysfunction. In neuroendocrine cells, PIKFYVE also negatively regulates calcium-dependent exocytosis. In addition, PIKFYVE has also been shown to phosphorylate Transcription Factor EB (TFEB), which may be related to the activity of PIKFYVE inhibitors in treating multiple myeloma.

PIKFYVE and PI(3,5)P2 have been linked to the pathogenesis of several diseases and disorders. PIKFYVE mutations are found in 8 out of 10 families with Francois-Neetens corneal fleck dystrophy. Interference with PIKFYVE function is associated with impaired glucose uptake. Studies in mice show that selective PIKFYVE disruption in skeletal muscle cells results in systemic insulin resistance, glucose intolerance, hyperinsulinemia and increased adiposity, all of which are signs of prediabetes in humans. This is further supported by studies showing that acute insulin treatment results in increases in PI(3,5)P2 concentration in adipocytes, and this promotes increased GLUT4 translocation and surface expression, increasing glucose transport into cells. In various other cell and animal models, PI(3,5)P2 has been shown to be elevated by hyperosmotic shock in adipocytes, mitogenic signals (such as IL-2 and UV light in lymphocytes), protein kinase C activation in platelets, and epidermal growth factor stimulation of COS cells.

Several small molecule inhibitors of PIKFYVE have recently been reported, including Apilimod. Apilimod, studied as an autoimmune disease treatment (Crohn's disease, rheumatoid arthritis) was originally identified as an inhibitor of IL-12 and IL-23 synthesis, but was later found to also have potent PIKFYVE inhibitory activity. It is suspected that Apilimod's activity may have been due to PIKFYVE inhibition rather than interreference with IL-12 and IL-23. PIKFYVE inhibitors have also shown promise as cancer therapies, in particular, for the treatment of non-Hodgkin lymphoma, multiple myeloma, melanoma, liver cancer, and glioblastoma. PIKFYVE inhibition has also shown promise as a therapy for amyotrophic lateral sclerosis (ALS) and fronto-temporal dementia (FTD), in particular ALS and FTD marked by repeat expansions of the C9ORF72 gene (C9FTD/ALS). PIKFYVE inhibition has also been suggested to be useful in the downstream inhibition of RANK signaling (receptor activator of nuclear factor kappa N), which plays an important role in bone remodeling and may be useful in treating bone resorption in multiple myeloma, prostate cancer and breast cancer patients.

Because of the critical role of intracellular vesicle trafficking in the life cycle of some eukaryotic viruses, PIKFYVE inhibitors also have been found effective in inhibiting viral infection. Enveloped viruses have a life cycle that begins with binding of a viral surface protein to a specific extracellular membrane protein on the target cell. For some viruses, such as HIV, receptor binding triggers fusion of the viral envelope with the cell membrane, resulting in deposition of the viral nucleoprotein complex into the cytoplasm. However, for other viruses, including Ebola, influenza A, vesicular stomatitis virus, Lassa fever virus, lymphocytic choriomeningitis virus, and coronaviruses (including MERS-CoV, SARS-CoV and SARS-CoV-2), receptor binding triggers endocytosis of the entire viral particle. The resulting endosome is transported within the cell until something triggers fusion of the viral envelope with the endosome membrane, resulting in deposition of the viral nucleoprotein complex into the cytoplasm. The triggering event can be acidification of the endosome or proteolysis of viral surface proteins.

Apilimod and other PIKFYVE inhibitors have been found to prevent infection by some of these enveloped viruses, either by interfering with endosome formation or by blocking endosome trafficking or otherwise preventing the triggering of endosome-viral envelope fusion.

There continues to be a need for new, selective inhibitors of PIKFYVE. The present disclosure provides novel, highly effective small-molecule inhibitors of PIKFYVE.

### SUMMARY OF THE INVENTION

Therefore, first aspect, the invention provides a compound of Formula I:

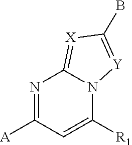

in free or pharmaceutically acceptable salt form, wherein
(i) X is —CH— or —N— (e.g., —CH—);
(ii) Y is —CH— or —N— (e.g., —N—);
(iii) A is an optionally substituted heteroaryl (e.g., 5-membered heteroaryl) or optionally substituted heterocycloalkyl (e.g., 3- to 6-membered heterocycloalkyl); (iv) B is halo, an optionally substituted aryl, optionally substituted heteroaryl, optionally substituted C₃₋₆cycloalkyl, optionally substituted 3- to 6-membered heterocycloalkyl, optionally substituted 3- to 6-membered heterocycloalkenyl, optionally substituted C₁₋₆alkyl, optionally substituted C₂₋₆alkenyl (e.g., vinyl), —N(Rₐ)—R₂, —O—R₂, —(CO)—R₂, —(CO)—O—R₂, —(CO)—N(Rₐ)—R₂, —O—(CO)—R₂, —N(Rₐ)—(CO)—R₂, —(CO)—N(Rₐ)—(CO)—R₂, N(Rₐ)—(CO)—N(Rₐ)—R₂, optionally substituted —(C₁₋₆alkyl)-(3- to 6-membered heterocycloalkyl), optionally substituted —(C₁₋₆alkyl)-(C₃₋₆cycloalkyl), optionally substituted —(C₁₋₆alkyl)-N(Rₐ)—R₂, optionally substituted —(C₁₋₆alkyl)-O—R₂, optionally substituted —(C₁₋₆alkyl)-(CO)—N(Rₐ)—R₂, optionally substituted —CH₂-(3- to 6-membered heterocycloalkyl), optionally substituted —C(O)-(3- to 6-membered heterocycloalkyl), optionally substituted —C(O)—(C₃₋₆cycloalkyl), optionally substituted —CH₂—(C₃₋₆cycloalkyl), —CH₂—N(Rₐ)—R₂, —CH₂—O—R₂, or —CH₂—(CO)—N(Rₐ)—R₂;
(v) R₁ is an optionally substituted C₁₋₆alkyl, optionally substituted C₃₋₆cycloalkyl, optionally substituted C₁₋₆alkoxy, optionally substituted 3- to 8-membered heterocycloalkyl (e.g., 3- to 7-membered or 3- to 6-membered heterocycloalkyl), —C(O)—R₂, —C(O)O—R₂, —OC(O)—R₂, —C(O)N(Rₐ)—R₂, —N(Rₐ)C(O)—R₂, —N(Rₐ)—R₂, or —O—R₂;
(vi) Rₐ is H, optionally substituted C₁₋₆alkyl, or optionally substituted C₃₋₆cycloalkyl; and
(vii) R₂ is optionally substituted C₁₋₆alkyl, optionally substituted C₃₋₆cycloalkyl, optionally substituted C₁₋₆alkoxy, optionally substituted aryl, optionally substituted heteroaryl, or optionally substituted 3- to 7-membered heterocycloalkyl;
   provided that:
      (a) R₁ is not optionally substituted piperazine when A is unsubstituted furan or thiophene, and B is optionally substituted phenyl;
      (b) R₁ is not optionally substituted piperazine when A is unsubstituted furan and B is unsubstituted furan or thiophene;
      (c) R₁ is not optionally substituted piperazine, morpholine or pyrrolidine, when A is unsubstituted pyridine and B is unsubstituted phenyl; and
      (d) R₁ is not unsubstituted morpholine or pyrrolidine when A is 3-methyl-2-quinoxalinyl and B is unsubstituted 1-pyrrolidinyl or 3-fluoro-1-pyrrolidinyl.

In a second aspect, the invention provides a pharmaceutical composition comprising the compound of Formula I, in free or pharmaceutically acceptable salt form, in admixture with a pharmaceutically acceptable diluent or carrier.

In a third aspect, the invention provides a method for the treatment or prophylaxis of a disease or disorder characterized by dysregulation of phosphoinositide-mediated signal transduction pathways or which may be ameliorated by modulating (e.g., inhibiting) PIKFYVE-dependent signaling pathways or by modulating (e.g., inhibiting) endosome formation or trafficking, comprising administering to a patient in need thereof a therapeutically effective amount of the compound of Formula I, in free or pharmaceutically acceptable salt form.

### DETAILED DESCRIPTION

In a first aspect, the invention provides a compound of Formula I:

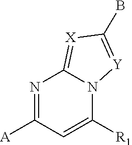

in free or pharmaceutically acceptable salt form, wherein
(i) X is —CH— or —N— (e.g., —CH—);
(ii) Y is —CH— or —N— (e.g., —N—);
(iii) A is an optionally substituted heteroaryl (e.g., 5-membered heteroaryl) or optionally substituted heterocycloalkyl (e.g., 3- to 6-membered heterocycloalkyl);
(iv) B is halo, an optionally substituted aryl, optionally substituted heteroaryl, optionally substituted C₃₋₆cycloalkyl, optionally substituted 3- to 6-membered heterocycloalkyl, optionally substituted 3- to 6-membered heterocycloalkenyl, optionally substituted C₁₋₆alkyl, optionally substituted C₂₋₆alkenyl (e.g., vinyl), —N(Rₐ)—R₂, —O—R₂, —(CO)—R₂, —(CO)—O—R₂, —(CO)—N(Rₐ)—R₂, —O—(CO)—R₂, —N(Rₐ)—(CO)—R₂, —(CO)—N(Rₐ)—(CO)—R₂, N(Rₐ)—(CO)—N(Rₐ)—R₂, optionally substituted —(C₁₋₆alkyl)-(3- to 6-membered heterocycloalkyl), optionally substituted —(C₁₋₆alkyl)-(C₃₋₆cycloalkyl), optionally substituted —(C₁₋₆alkyl)-N(Rₐ)—R₂, optionally substituted —(C₁₋₆alkyl)-O—R₂, optionally substituted —(C₁₋₆alkyl)-(CO)—N(Rₐ)—R₂, optionally substituted —CH₂-(3- to 6-membered heterocycloalkyl), optionally substituted —C(O)-(3- to 6-membered heterocycloalkyl), optionally substituted —C(O)—(C₃₋₆cycloalkyl), optionally substituted —CH₂—(C₃₋₆cycloalkyl), —CH₂—N(Rₐ)—R₂, —CH₂—O—R₂, or —CH₂—(CO)—N(Rₐ)—R₂;
(v) R₁ is an optionally substituted C₁₋₆alkyl, optionally substituted C₃₋₆cycloalkyl, optionally substituted C₁₋₆alkoxy, optionally substituted 3- to 8-membered heterocycloalkyl (e.g., 3- to 7-membered or 3- to 6-membered heterocycloalkyl), —C(O)—R₂, —C(O)O—R₂, —OC(O)—R₂, —C(O)N(Rₐ)—R₂, —N(Rₐ)C(O)—R₂, —N(Rₐ)—R₂, or —O—R₂;
(vi) Rₐ is H, optionally substituted C₁₋₆alkyl, or optionally substituted C₃₋₆cycloalkyl; and
(vii) R₂ is optionally substituted C₁₋₆alkyl, optionally substituted C₃₋₆cycloalkyl, optionally substituted C₁₋₆alkoxy, optionally substituted aryl, optionally substituted heteroaryl, or optionally substituted 3- to 7-membered heterocycloalkyl;
   provided that:
      (a) R₁ is not optionally substituted piperazine when A is unsubstituted furan or thiophene, and B is optionally substituted phenyl;
      (b) R₁ is not optionally substituted piperazine when A is unsubstituted furan and B is unsubstituted furan or thiophene;
      (c) R₁ is not optionally substituted piperazine, morpholine or pyrrolidine, when A is unsubstituted pyridine and B is unsubstituted phenyl; and
      (d) R₁ is not unsubstituted morpholine or pyrrolidine when A is 3-methyl-2-quinoxalinyl and B is unsubstituted 1-pyrrolidinyl or 3-fluoro-1-pyrrolidinyl.

In particular embodiments, the invention provides a compound according to the following Formulas:
1.1 The compound of Formula I, wherein X is —N— and Y is —CH;
1.2 The compound of Formula I, wherein X is —CH— and Y is —CH—;
1.3 The compound of Formula I, wherein X is —N— and Y is —N—;
1.4 The compound of Formula I, wherein X is —CH— and Y is —N—;
1.5 The compound of Formula I or any of 1.1-1.4, wherein A is an optionally substituted heteroaryl;
1.6 The compound of Formula 1.5, wherein said heteroaryl is selected from pyridine, pyrimidine, pyridazine, pyrazine, triazine, thiophene, furan, pyrrole, oxazole, imidazole, thiazole, pyrazole, isoxazole, isothiazole, triazole (e.g., 1,2,3-triazole, or 1,2,4-triazole), oxadiazole (e.g., 1,2,3-oxadiazole, or 1,2,4-oxadiazole), thiadiazole (e.g., 1,2,3-thiadiazole, or 1,2,4-thiadiazole), tetrazole (e.g., 1,2,3,4-tetrazole), and indole;
1.7 The compound of Formula 1.5, wherein said heteroaryl is selected from pyrrole, oxazole, imidazole, thiazole, pyrazole, isoxazole, isothiazole, indole, benzimidazole, benzoxazole, benzothiazole, indazole, benzisoxazole, and benzisothiazole;
1.8 The compound of Formula 1.5, wherein said heteroaryl is pyrazole (e.g., 3-substituted-1-pyrazolyl or 1-substituted-3-pyrazolyl);
1.9 Any of Compounds 1.1-1.8, wherein said heteroaryl is substituted with one or more groups selected from OH, CN, C₁₋₆alkyl (e.g., methyl), halogen (e.g., F), C₁₋₆alkoxy (e.g., methoxy), hydroxyC₁₋₆alkyl (e.g., 2-hydroxyethyl or 1-hydroxyethyl), C₁₋₆alkyl-C₁₋₆alkoxy (e.g., methoxymethyl or ethoxymethyl), C₁₋₆alkyl-C₁₋₆thioalkyl (e.g., methylthiomethyl), haloC₁₋₆alkyl (e.g., CHF₂ or CF₃), haloC₁₋₆alkoxy (e.g., OCF₃), carboxy (COOH), aryl, heteroaryl, C₃₋₆cycloalkyl, 3- to 10-membered heterocycloalkyl (e.g., 3- to 6-membered heterocycloalkyl, such as 1-piperidinyl or 3-piperidinyl), and 3- to 10-membered heterocycloalkenyl (e.g., 3- to 6-membered heterocycloalkenyl, such as 1,2,3,6-tetrahydropyridin-1-yl or 1,2,3,6-tetrahydropyridin-3-yl), wherein said alkyl, alkoxy, thioalkyl, aryl, heteroaryl, cycloalkyl, heterocycloalkyl, and heterocycloalkenyl, are each optionally independently substituted with one or more groups selected from OH, CN, C₁₋₆alkyl (e.g., methyl or t-butyl), halogen (e.g., F or Br), C₁₋₆alkoxy (e.g., methoxy, ethoxy), haloC₁₋₆alkyl (e.g., CF₃), haloC₁₋₆alkoxy (e.g., OCF₃), carboxy (COOH), C₃₋₆cycloalkyl, and 5- or 6-membered heterocycloalkyl;
1.10 The compound of Formula 1.9, wherein said heteroaryl is substituted with aryl (e.g., phenyl) or heteroaryl (e.g., pyridyl or pyrimidinyl), wherein said aryl or heteroaryl is optionally substituted with one or more groups selected from OH, CN, C₁₋₆alkyl (e.g., methyl or t-butyl), halogen (e.g., F or Br), C₁₋₆alkoxy (e.g., methoxy or ethoxy), hydroxyC₁₋₆alkyl (e.g., 2-hydroxyethyl or 1-hydroxyethyl), C₁₋₆alkyl-C₁₋₆alkoxy (e.g., methoxymethyl or ethoxymethyl), C₁₋₆alkyl-C₁₋₆thioalkyl (e.g., methylthiomethyl), haloC₁₋₆alkoxy (e.g., OCF₃), and haloC₁₋₆alkyl (e.g., CHF₂ or CF₃);
1.11 The compound of Formula 1.10, wherein said heteroaryl is substituted with an optionally substituted heteroaryl selected from pyridyl, pyrimidinyl, pyrazinyl, pyridazinyl, indolyl (e.g., indol-1-yl, or indol-3-yl), indazolyl (e.g., indazol-1-yl, or indazol-3-yl), benzimidazolyl (e.g., benzimidazol-1-yl, or benzimidazol-2-yl), benzisoxazolyl (e.g., benzisoxazol-3-yl), benzisothiazolyl (e.g., benzisothiazol-3-yl), benzoxazolyl (e.g., benzoxazol-2-yl), benzothiazolyl (e.g., benzothiazol-2-yl), thiazolyl (e.g., 4-thiazolyl or 2-thiazolyl), and pyrazolyl (e.g., 4-pyrazolyl);
1.12 The compound of Formula 1.10, wherein said heteroaryl is substituted with unsubstituted phenyl or C₁₋₆alkyl (e.g., methyl or t-butyl);
1.13 The compound of Formula 1.10, wherein said heteroaryl is substituted with phenyl substituted with one, two or three groups independently selected from CN, C₁₋₆alkyl (e.g., methyl or t-butyl), halogen (e.g., F or Br), C₁₋₆alkoxy (e.g., methoxy or ethoxy), hydroxyC₁₋₆alkyl (e.g., 2-hydroxyethyl or 1-hydroxyethyl), C₁₋₆alkyl-C₁₋₆alkoxy (e.g., methoxymethyl or ethoxymethyl), haloC₁₋₆alkoxy (e.g., OCF₃), and haloC₁₋₆alkyl (e.g., CF₃);
1.14 The compound of Formula 1.13, wherein said heteroaryl is 3-substituted-pyrazol-1-yl or 1-substituted-pyrazol-3-yl, and said substituent is phenyl substituted with one, two or three groups independently selected from CN, C₁₋₆alkyl (e.g., methyl or t-butyl), halogen (e.g., F or Br), C₁₋₆alkoxy (e.g., methoxy or ethoxy), hydroxyC₁₋₆alkyl (e.g., 2-hydroxyethyl or 1-hydroxyethyl), C₁₋₆alkyl-C₁₋₆alkoxy (e.g., methoxymethyl or ethoxymethyl), haloC₁₋₆alkoxy (e.g., OCF₃), and haloC₁₋₆alkyl (e.g., CF₃);
1.15 The compound Formula 1.13, wherein said heteroaryl is 3-substituted-pyrazol-1-yl or 1-substituted-pyrazol-3-yl, and said substituent is phenyl substituted with one group selected from CN, C₁₋₆alkyl (e.g., methyl or t-butyl), halogen (e.g., F or Br), C₁₋₆alkoxy (e.g., methoxy or ethoxy), hydroxyC₁₋₆alkyl (e.g., 2-hydroxyethyl or 1-hydroxyethyl), C₁₋₆alkyl-C₁₋₆alkoxy (e.g., methoxymethyl or ethoxymethyl), haloC₁₋₆alkoxy (e.g., OCF₃), and haloC₁₋₆alkyl (e.g., CF₃);
1.16 The compound of Formula 1.15, wherein said phenyl is meta-substituted;
1.17 The compound of Formula 1.15, wherein said phenyl is substituted with one group selected from methyl, bromo, chloro, fluoro, methoxy, ethoxy, trifluoromethyl, and trifluoromethoxy.
1.18 The compound of Formula 1.15, wherein substituent A is 3-(3-methoxyphenyl)-pyrazol-1-yl, 3-(3-tolyl)-pyrazol-1-yl, 3-(3-bromophenyl)-pyrazol-1-yl, 3-(3-fluorophenyl)-pyrazol-1-yl, 3-(3-ethoxyphenyl)-pyrazol-1-yl 3-(3-trifluoromethylphenyl)-pyrazol-1-yl, or 3-(3-trifluoromethoxyphenyl)-pyrazol-1-yl;
1.19 The compound of Formula I or any of 1.1-1.4, wherein A is an optionally substituted heterocycloalkyl;
1.20 The compound of Formula 1.19, wherein said heterocycloalkyl is selected from aziridine, azetidine, oxetane, pyrrolidine, tetrahydrofuran, tetrahydropyran, morpholine, piperidine, and piperazine;
1.21 The compound of Formula 1.19, wherein said heterocycloalkyl is pyrrolidine;
1.22 Any of Compounds 1.19-1.21, wherein said heterocycloalkyl is substituted with one or more groups selected from OH, CN, C₁₋₆alkyl (e.g., methyl), halogen (e.g., F), C₁₋₆alkoxy (e.g., methoxy), haloC₁₋₆alkyl (e.g., CF₃), haloC₁₋₆alkoxy (e.g., OCF₃), carboxy (COOH), aryl, heteroaryl, C₃₋₆cycloalkyl, and 3- to 6-membered heterocycloalkyl, wherein said alkyl, alkoxy, aryl, heteroaryl, cycloalkyl, and heterocycloalkyl is each optionally independently substituted with one or more groups selected from OH, CN, C₁₋₆alkyl (e.g., methyl or t-butyl), halogen (e.g., F or Br), C₁₋₆alkoxy (e.g., methoxy), haloC₁₋₆alkyl (e.g., CF₃), haloC₁₋₆alkoxy (e.g., OCF₃), carboxy (COOH), C₃₋₆cycloalkyl, and 5- or 6-membered heterocycloalkyl;
1.23 The compound of Formula 1.22, wherein said heterocycloalkyl is substituted with aryl (e.g., phenyl) or heteroaryl (e.g., pyridyl or pyrimidinyl), wherein said aryl or heteroaryl is optionally substituted with one or more groups selected from OH, CN, C₁₋₆alkyl (e.g., methyl or t-butyl), halogen (e.g., F or Br), C₁₋₆alkoxy (e.g., methoxy), haloC₁₋₆alkoxy (e.g., OCF₃), and haloC₁₋₆alkyl (e.g., CF₃);
1.24 The compound of Formula 1.23, wherein said heterocycloalkyl is substituted with unsubstituted phenyl;
1.25 The compound of Formula 1.23, wherein said heterocycloalkyl is substituted with phenyl substituted with one, two or three groups independently selected from CN, C₁₋₆alkyl (e.g., methyl or t-butyl), halogen (e.g., F or Br), C₁₋₆alkoxy (e.g., methoxy), haloC₁₋₆alkoxy (e.g., OCF₃), and haloC₁₋₆alkyl (e.g., CF₃);
1.26 The compound of Formula I, or any of 1.1-1.25, wherein B is selected from halo (e.g., bromo), phenyl, pyridine, pyrimidine, pyridazine, pyrazine, triazine, thiophene, furan, pyrrole, oxazole, imidazole, thiazole, pyrazole, isoxazole, isothiazole, indazole, benzimidazole, benzisoxazole, benzisothiazole, benzoxazole, benzothiazole, and indole, each optionally an N-oxide thereof (e.g., pyridyl-N-oxide), and each optionally substituted;
1.27 The compound of Formula 1.26, wherein B is selected from phenyl, pyridine, pyrimidine, pyridazine, and pyrazine, each optionally substituted;
1.28 The compound of Formula 1.26, wherein B is selected from pyridine, pyridazine, and pyrimidine, each optionally substituted;
1.29 The compound of Formula 1.26, wherein B is pyridine, e.g., 2-pyridinyl, 3-pyridinyl, or 4-pyridinyl, or 4-pyridinyl-N-oxide, each optionally substituted;
1.30 The compound of Formula I, or any of 1.1-1.25, wherein B is heterocycloalkyl or heterocycloalkenyl, each optionally substituted;
1.31 The compound of Formula 1.30, wherein B is selected from morpholine, piperidine, 1,2,3,6-tetrahydropyridinyl, piperazine, tetrahydropyran, pyrrolidine, tetrahydrofuran, oxetane, azetidine, oxirane, and aziridine, each optionally substituted (e.g., with C₁₋₆alkyl (e.g., N-substituted));
1.32 The compound of Formula I, or any of 1.1-1.25, wherein B is optionally substituted —CH₂-(3- to 6-membered heterocycloalkyl) or optionally substituted —C(O)-(3- to 6-membered heterocycloalkyl);
1.33 The compound of Formula 1.32, wherein B is optionally substituted —CH₂-(3- to 6-membered heterocycloalkyl) or optionally substituted —C(O)-(3- to 6-membered heterocycloalkyl) and said heterocycloalkyl is selected from morpholine, piperidine, piperazine, tetrahydropyran, pyrrolidine, tetrahydrofuran, oxetane, azetidine, oxirane, and aziridine, each optionally substituted (e.g., spiro-substituted);
1.34 The compound of Formula 1.33, wherein B is selected from —CH₂-(morpholine), —CH₂-(piperidine), —CH₂-(piperazine), —CH₂-(pyrrolidine), —C(O)-(morpholine), —C(O)-(piperidine), —C(O)-(piperazine), and —C(O)-(pyrrolidine), each optionally substituted (e.g., spiro substituted);
1.35 The compound of Formula I, or any of 1.1-1.25, wherein B is optionally substituted C₁₋₆alkyl (e.g., wherein B is methyl, ethyl, propyl, isopropyl n-butyl, s-butyl, or t-butyl, optionally further substituted by methyl, ethyl, etc.);
1.36 The compound of Formula I, or any of Compounds 1.1-1.35, wherein substituent B is substituted with one or more groups selected from OH, CN, C₁₋₆alkyl (e.g., methyl), halogen (e.g., F), C₁₋₆alkoxy (e.g., methoxy), haloC₁₋₆alkyl (e.g., CF₃), haloC₁₋₆alkoxy (e.g., OCF₃), carboxy (COOH), C₃₋₆cycloalkyl (e.g., cyclopropyl), —NH(C₁₋₆alkyl), —N(C₁₋₆alkyl)(C₁₋₆alkyl), —SO—C₁₋₆alkyl, —SO₂—C₁₋₆alkyl, —NH—SO₂—(C₁₋₆alkyl), —N(C₁₋₆alkyl)—SO₂—(C₁₋₆alkyl), —NH—C(O)—(C₁₋₆alkyl), —N(C₁₋₆alkyl)-C(O)—(C₁₋₆alkyl), —(C₁₋₆alkyl)-C(O)NH₂, —(C₁₋₆alkyl)-C(O)NH(C₁₋₆alkyl), —(C₁₋₆alkyl)-C(O)NH(C₁₋₆alkyl)(C₁₋₆alkyl), aryl, heteroaryl, C₃₋₆cycloalkyl, 3- to 6-membered heterocycloalkyl (e.g., morpholine), spiro-3-6-membered heterocycloalkyl (e.g., spiro-oxetane), wherein said alkyl, alkoxy, aryl, heteroaryl, cycloalkyl, and heterocycloalkyl is each optionally independently substituted with one or more groups selected from OH, CN, C₁₋₆alkyl (e.g., methyl or t-butyl), halogen (e.g., F or Br), haloC₁₋₆alkoxy (e.g., OCF₃), C₁₋₆alkoxy (e.g., methoxy), haloC₁₋₆alkyl (e.g., CF₃), carboxy (COOH), C₃₋₆cycloalkyl (e.g., cyclopropyl), and 3- to 6-membered heterocycloalkyl (e.g., morpholine, oxetane);
1.37 The compound of Formula 1.36, wherein substituent B is substituted with one group selected from halo (e.g., F), C₁₋₆alkyl (e.g., methyl), —NH(C₁₋₆alkyl), —N(C₁₋₆alkyl)(C₁₋₆alkyl), —SO—C₁₋₆alkyl, —SO₂—C₁₋₆alkyl, —NH—SO₂—(C₁₋₆alkyl), —N(C₁₋₆alkyl)-SO₂—(C₁₋₆alkyl), —NH—C(O)—(C₁₋₆alkyl), —(C₁₋₆alkyl)-C(O)NH₂, —(C₁₋₆alkyl)-C(O)NH(C₁₋₆alkyl), —(C₁₋₆alkyl)-C(O)NH(C₁₋₆alkyl)(C₁₋₆alkyl), —N(C₁₋₆alkyl)-C(O)—(C₁₋₆alkyl), spiro-3-6-membered heterocycloalkyl (e.g., spiro-oxetane), wherein said alkyl each optionally independently substituted with one or more groups selected from OH and halogen;
1.38 The compound of Formula 1.37, wherein each of said C₁₋₆alkyl is methyl or ethyl;
1.39 The compound of any one of Formulas 1.32-1.38, wherein group B is unsubstituted —CH₂-(morpholine), unsubstituted —C(O)-(morpholine), or —CH₂-(piperidine), —CH₂-(piperazine), —CH₂-(pyrrolidine), C(O)-(piperidine), —C(O)-(piperazine), or —C(O)-(pyrrolidine), each unsubstituted or substituted by one group selected from the group consisting of halo (e.g., fluoro), C₁₋₆alkyl (e.g., methyl, isopropyl), —N(C₁₋₆alkyl)(C₁₋₆alkyl) (e.g., —NMe₂), —SO₂—C₁₋₆alkyl (e.g., —SO₂-Me), —N(C₁₋₆alkyl)-SO₂—(C₁₋₆alkyl) (e.g., —N(Me)-SO₂-Me), —N(C₁₋₆alkyl)-C(O)—(C₁₋₆alkyl) (e.g., —N(Me)-C(O)-Me), C₁₋₆alkyl substituted by OH (e.g., —C(Me)₂OH), C₃₋₆cycloalkyl (e.g., cyclopropyl), 3-6 membered heterocycloalkyl (e.g., morpholine, oxetane), —(C₁₋₆alkyl)-C(O)—NH₂ (e.g., —N(Me)-C(O)—NH₂), and spiro-3-6-membered heterocycloalkyl (e.g., spiro-oxetane);
1.40 The compound of any one of Formulas 1.26-1.39, wherein group B is selected from the group consisting of:

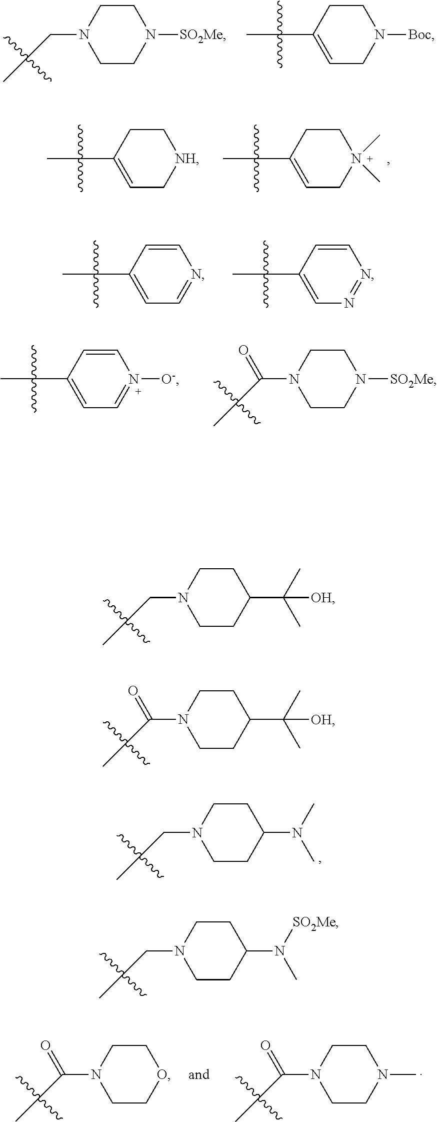

1.41 The compound of Formula I, or any of Compounds 1.1-1.35, wherein substituent B is unsubstituted;
1.42 The compound of Formula I, or any of Compounds 1.1-1.41, wherein R₁ is C₁₋₆alkyl, C₃₋₆cycloalkyl, or C₁₋₆alkoxy, each substituted with an optionally substituted 3- to 8-membered heterocycloalkyl;
1.43 The compound of Formula I, or any of Compounds 1.1-1.41, wherein R₁ is —C(O)—R₂, —C(O)O—R₂, —OC(O)—R₂, —C(O)N(Rₐ)—R₂, —N(Rₐ)C(O)—R₂, —N(Rₐ)—R₂, or —O—R₂; and wherein R₂ is an optionally substituted 3- to 8-membered heterocycloalkyl;
1.44 The compound of Formula I, or any of Compounds 1.1-1.41, wherein R₁ is an optionally substituted 3- to 8-membered heterocycloalkyl;
1.45 The compound of any of Formulas 1.42-1.44, wherein said 3- to 8-membered heterocycloalkyl is selected from aziridine, azetidine (e.g., azetidine-3-one), oxetane, pyrrolidine (e.g., 3,3-difluoropyrrolidin-1-yl), pyrrolidinone (e.g., 1-pyrrolidin-3-one), tetrahydrofuran, dihydropyran, tetrahydropyran, morpholine, piperidine (e.g., 4,4-difluoropiperidine), piperazine, oxazepane (e.g., 1,4-oxazepane), hexahydro-1H-furo[3,4-c]pyrrole, and oxa-azaspiro[3.3]heptane (e.g., 2-oxa-6-azaspiro[3.3]heptan-6-yl), each optionally substituted;
1.46 The compound of Formula 1.45, wherein said heterocycloalkyl is morpholine (e.g., 2-methyl-4-morpholinyl, or 3-methyl-4-morpholinyl, or 4-morpholinyl (i.e., N-morpholinyl));
1.47 The compound of Formula 1.45, wherein said heterocycloalkyl is selected from aziridine, azetidine (e.g., azetidine-3-one or azetidine-3-spiro-oxetane), pyrrolidine (e.g., 3,3-difluoropyrrolidin-1-yl), piperidine (e.g., 4,4-difluoropiperidin-1-yl), 1-pyrrolidin-3-one, and 2-oxa-6-azaspiro[3.3]heptan-6-yl;
1.48 The compound of any of Formulas 1.42-1.47, wherein said heterocycloalkyl is substituted with one or more groups selected from OH, CN, C₁₋₆alkyl (e.g., methyl), halogen (e.g., F), C₁₋₆alkoxy (e.g., methoxy), haloC₁₋₆alkyl (e.g., CF₃), carboxy (COOH), and 3-6 membered heterocycloalkyl (e.g., morpholine, oxetane, optionally spiro connected);
1.49 The compound of any of Formulas 1.42-1.47, wherein said heterocycloalkyl (e.g., 4-morpholinyl) is unsubstituted;
1.50 The compound according to Formula I or any of Formulas 1.1-1.49, wherein the compound is selected from:

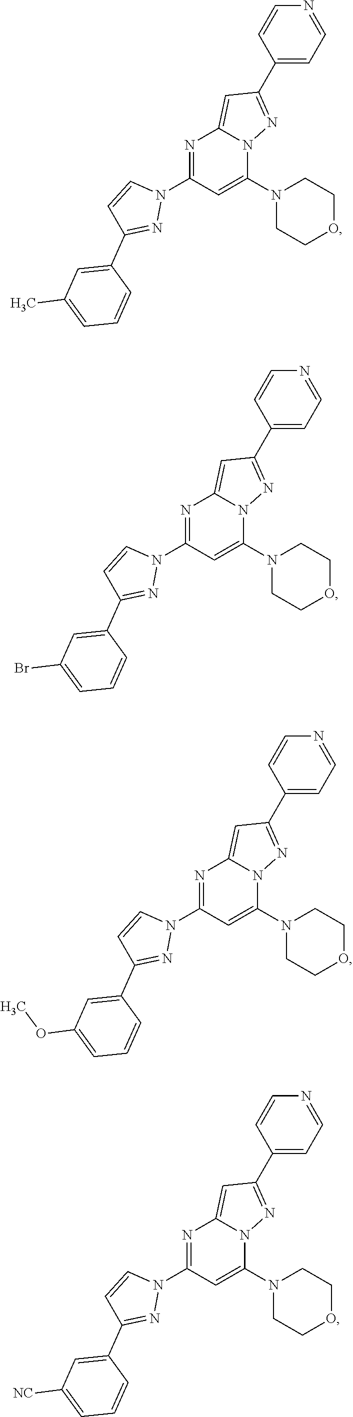 [SMILES: Cc1cccc(-c2ccn(-c3cc(N4CCOCC4)n4nc(-c5ccncc5)cc4n3)n2)c1 ; Brc1cccc(-c2ccn(-c3cc(N4CCOCC4)n4nc(-c5ccncc5)cc4n3)n2)c1 ; COc1cccc(-c2ccn(-c3cc(N4CCOCC4)n4nc(-c5ccncc5)cc4n3)n2)c1 ; N#Cc1cccc(-c2ccn(-c3cc(N4CCOCC4)n4nc(-c5ccncc5)cc4n3)n2)c1]

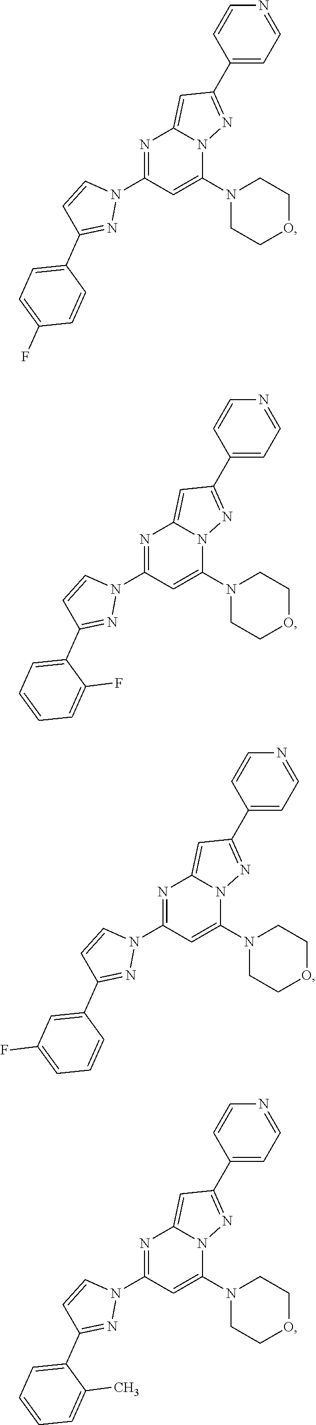 [SMILES: Fc1ccc(-c2ccn(-c3cc(N4CCOCC4)n4nc(-c5ccncc5)cc4n3)n2)cc1 ; Fc1ccccc1-c1ccn(-c2cc(N3CCOCC3)n3nc(-c4ccncc4)cc3n2)n1 ; Fc1cccc(-c2ccn(-c3cc(N4CCOCC4)n4nc(-c5ccncc5)cc4n3)n2)c1 ; Cc1ccccc1-c1ccn(-c2cc(N3CCOCC3)n3nc(-c4ccncc4)cc3n2)n1]

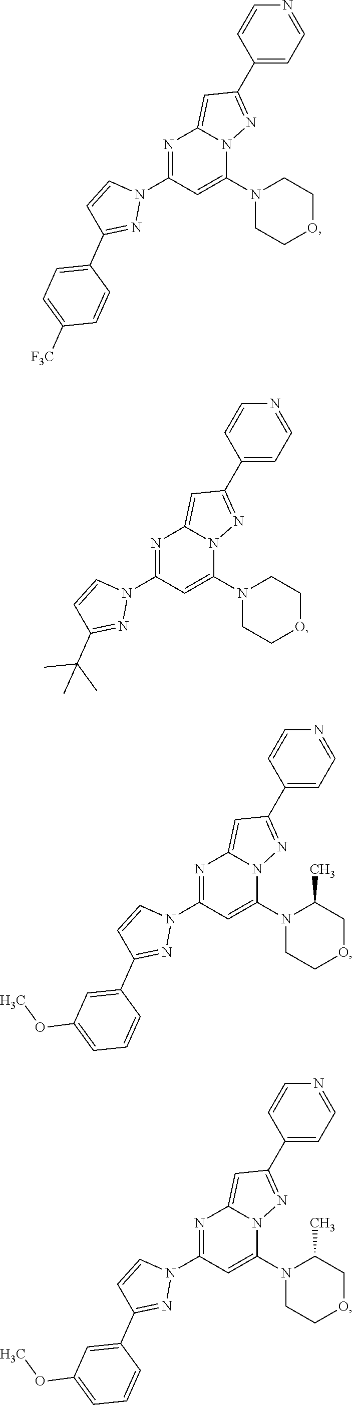 [SMILES: FC(F)(F)c1ccc(-c2ccn(-c3cc(N4CCOCC4)n4nc(-c5ccncc5)cc4n3)n2)cc1 ; CC(C)(C)c1ccn(-c2cc(N3CCOCC3)n3nc(-c4ccncc4)cc3n2)n1 ; COc1cccc(-c2ccn(-c3cc(N4CCOC[C@@H]4C)n4nc(-c5ccncc5)cc4n3)n2)c1 ; COc1cccc(-c2ccn(-c3cc(N4CCOC[C@H]4C)n4nc(-c5ccncc5)cc4n3)n2)c1]

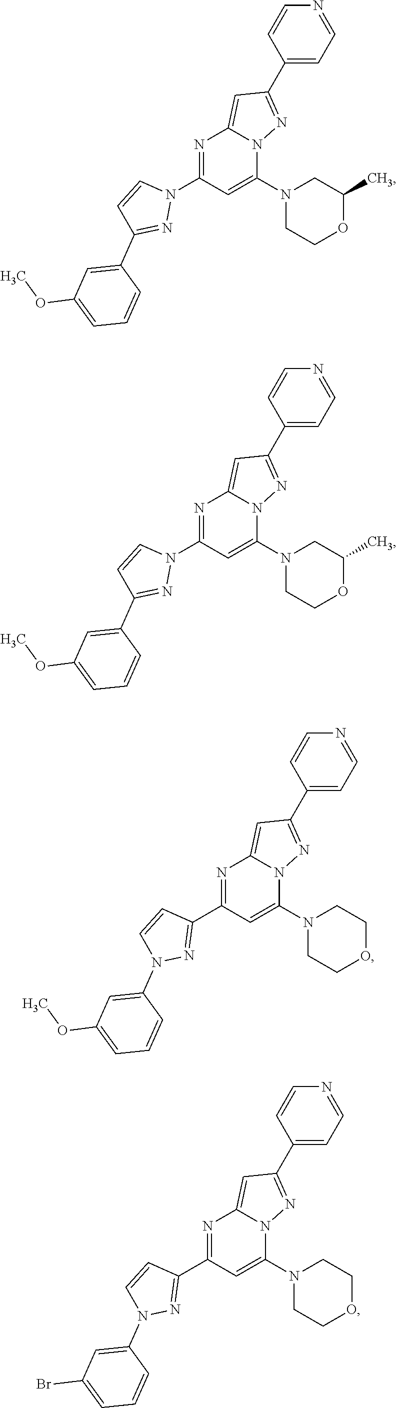 [SMILES: COc1cccc(-c2ccn(-c3cc(N4CCO[C@H](C)C4)n4nc(-c5ccncc5)cc4n3)n2)c1 ; COc1cccc(-c2ccn(-c3cc(N4CCO[C@@H](C)C4)n4nc(-c5ccncc5)cc4n3)n2)c1 ; COc1cccc(-n2ccc(-c3cc(N4CCOCC4)n4nc(-c5ccncc5)cc4n3)n2)c1 ; Brc1cccc(-n2ccc(-c3cc(N4CCOCC4)n4nc(-c5ccncc5)cc4n3)n2)c1]

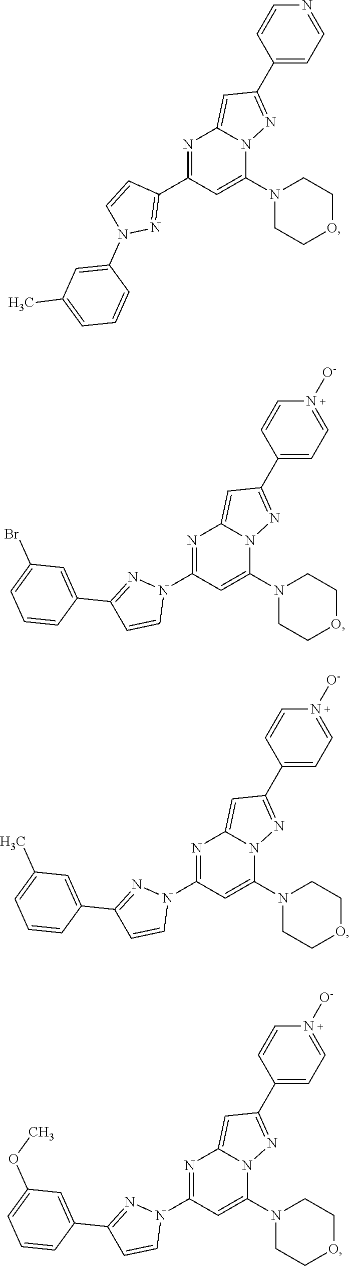

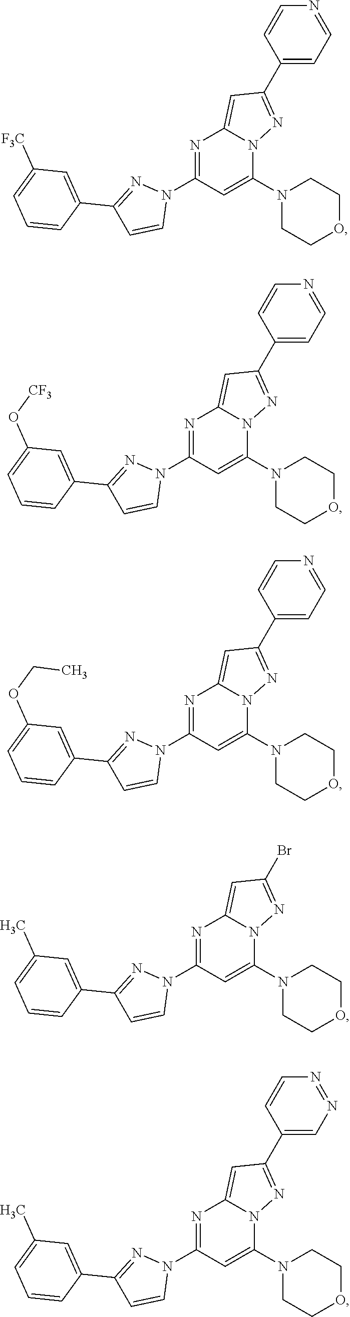

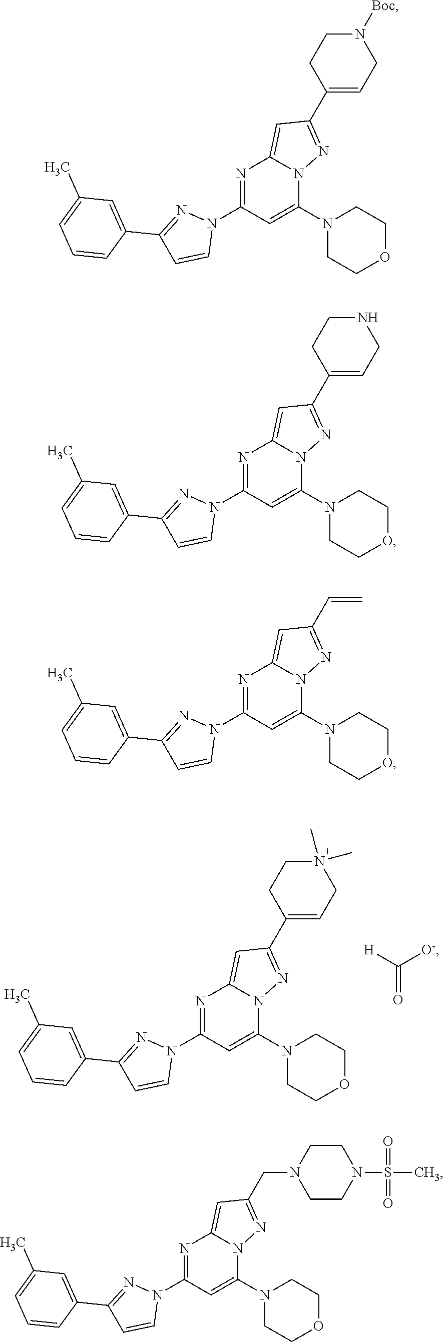

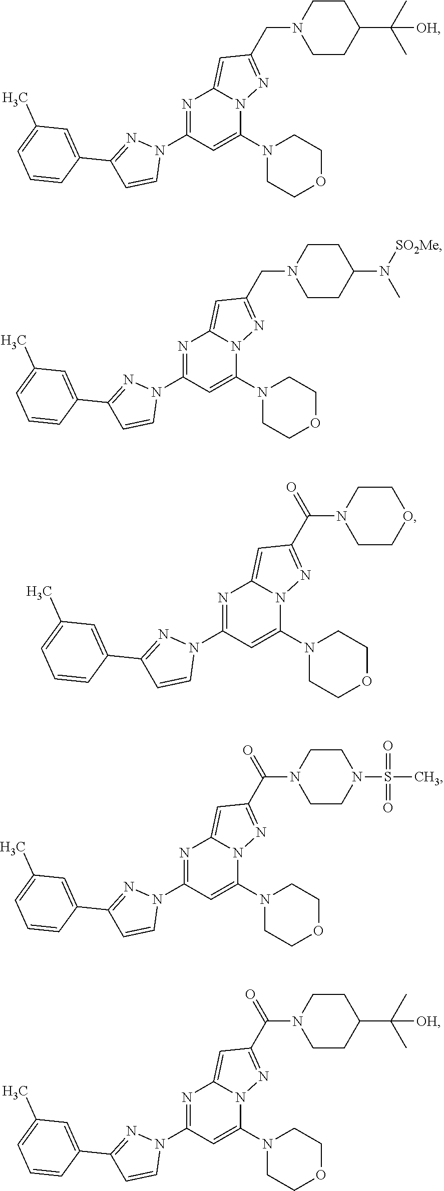 [SMILES: Cc1cccc(-c2ccn(-c3cc(N4CCOCC4)n4nc(CN5CCC(C(C)(C)O)CC5)cc4n3)n2)c1 ; Cc1cccc(-c2ccn(-c3cc(N4CCOCC4)n4nc(CN5CCC(N(C)S(C)(=O)=O)CC5)cc4n3)n2)c1 ; Cc1cccc(-c2ccn(-c3cc(N4CCOCC4)n4nc(C(=O)N5CCOCC5)cc4n3)n2)c1 ; Cc1cccc(-c2ccn(-c3cc(N4CCOCC4)n4nc(C(=O)N5CCN(S(C)(=O)=O)CC5)cc4n3)n2)c1 ; Cc1cccc(-c2ccn(-c3cc(N4CCOCC4)n4nc(C(=O)N5CCC(C(C)(C)O)CC5)cc4n3)n2)c1]

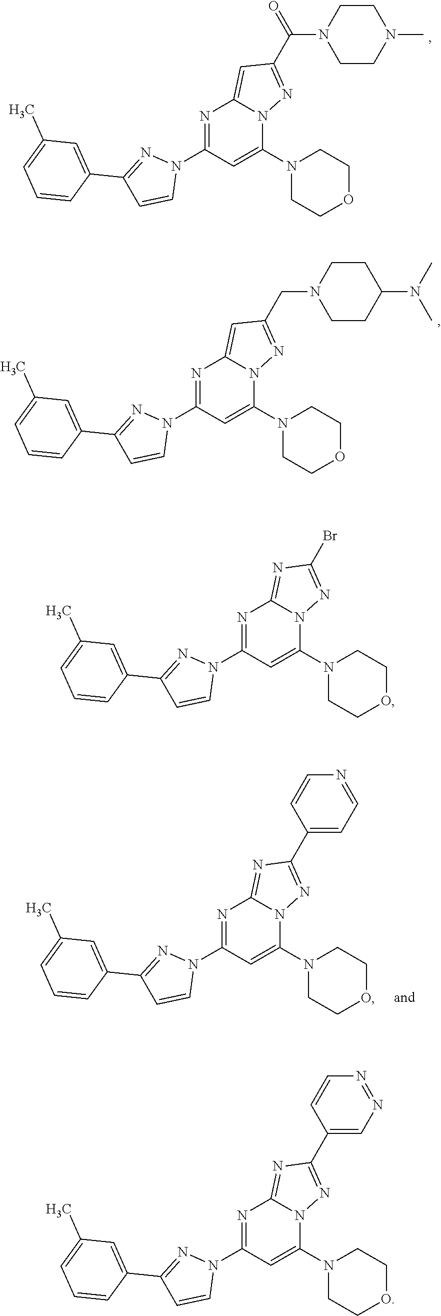 [SMILES: Cc1cccc(-c2ccn(-c3cc(N4CCOCC4)n4nc(C(=O)N5CCN(C)CC5)cc4n3)n2)c1 ; Cc1cccc(-c2ccn(-c3cc(N4CCOCC4)n4nc(CN5CCC(N(C)C)CC5)cc4n3)n2)c1 ; Cc1cccc(-c2ccn(-c3cc(N4CCOCC4)n4nc(Br)nc4n3)n2)c1 ; Cc1cccc(-c2ccn(-c3cc(N4CCOCC4)n4nc(-c5ccncc5)nc4n3)n2)c1 ; Cc1cccc(-c2ccn(-c3cc(N4CCOCC4)n4nc(-c5ccnnc5)nc4n3)n2)c1]

1.51 The compound according to Formula I or any of Formulas 1.1-1.49, wherein the compound is selected from any one or more of the compounds of Examples 1 to 423.
1.52 The compound according to Formula I or any of Formulas 1.1-1.49, wherein the compound has any combination of variables X, Y, A, B and R₁ appearing in Table 1.
1.53 The compound according to Formula I or any of Formulas 1.1-1.52, wherein the compound is in free base form;
1.54 The compound according to Formula I or any of Formulas 1.1-1.52, wherein the compound is in the form of a pharmaceutically acceptable acid addition salt (e.g., hydrochloride);
1.55 The compound according to Formula I or any of Formulas 1.1-1.54, wherein the compound is an inhibitor of PIKFYVE (e.g., the compound having a Ki or IC₅₀ of less than 10 μM, or less than 1 μM, or less than 100 nM, or less than 50 nM, or less than 25 nM, or less than 10 nM; and/or the compound provides >50% inhibition at a concentration of 1 puM, or >75%, or >85% or >90% inhibition at said concentration;
in free or pharmaceutically acceptable salt form.

In a second aspect, the invention provides a pharmaceutical composition comprising the compound of Formula I or any of 1.1-1.55 as described herein, in free or pharmaceutically acceptable salt form, in admixture with a pharmaceutically acceptable diluent or carrier. In some embodiments, the composition is a composition for oral administration, such as a tablet or capsule. In some embodiments, such an oral dosage form is an immediate-release composition, or a delayed release composition, or a sustained release composition. In other embodiments, the pharmaceutical composition is an injectable composition, such as for intravenous, intramuscular, intrathecal, intraabdominal, intraperitoneal, or subcutaneous injection. In other embodiments, the pharmaceutical composition may be an inhalational composition, including powdered and aerosol compositions (i.e., gas liquid/emulsions), such as an intranasal composition (e.g., spray) or an intrapulmonary composition (e.g., metered dose inhaler).

Pharmaceutical compositions include all compositions wherein the compounds of the present invention are contained in an amount that is effective to achieve its intended purpose. While individual needs vary, determination of optimal ranges of effective amounts of each component is within the skill of the art. Preferably, the compounds may be administered to mammals, e.g., humans, orally at a dose of 0.0025 to 50 mg/kg, or an equivalent amount of the pharmaceutically acceptable salt thereof, per day of the body weight of the mammal being treated. For intramuscular injection, the dose is generally about one-half of the oral dose.

The unit oral dose may comprise from about 0.01 to about 1000 mg, preferably about 0.1 to about 100 mg of the compound, or 0.1 to 50 mg. The unit dose may be administered one or more times daily as needed to achieve the desired intended daily dosage.

The compounds of Formula I or any of 1.1-1.55 as described herein are highly effective inhibitors of PIKFYVE, preferably producing inhibition at nanomolar concentrations. In some embodiments the compounds are selective PIKFYVE inhibitors, e.g., the compounds have little or no inhibitory activity of other kinases, for example, other lipid kinases (e.g., other phosphoinositide kinases, such as phosphoinositide 3-kinases, phosphoinositide 4-kinases, phosphoinositide 5-kinases, phosphoinositide-5-phosphate 4-kinases, and phosphatidyl inositol 4-phosphate 5-kinases), and protein kinases (e.g., tyrosine kinases and serine-threonine kinases). In some embodiments, the compounds have a Kd or IC₅₀ of greater than 100 nM, or greater than 500 nM, or greater than 1000 nM, or greater than 10,000 nM, or greater than 50,000 nM against one or more of these other kinases, and/or the compound provides <50% inhibition at a concentration of 1 μM, or <25%, or <10%% or <5%, or <1% inhibition at said concentration against one or more of these other kinases.

PIKFYVE inhibitors according to the invention are therefore useful for treatment and prophylaxis of diseases and disorders which may be ameliorated by modulating (e.g., inhibiting) PIKFYVE-dependent signaling pathways or by modulating (e.g., inhibiting) endosome formation or trafficking.

Therefore, in the third aspect, the invention provides a method for the treatment or prophylaxis of a disease or disorder characterized by dysregulation of phosphoinositide-mediated signal transduction pathways or which may be ameliorated by modulating (e.g., inhibiting) PIKFYVE-dependent signaling pathways or by modulating (e.g., inhibiting) endosome formation or trafficking, comprising administering to a patient in need thereof a therapeutically effective amount of the compound of Formula I, or any of formulae 1.1-1.55 as described herein, in free or pharmaceutically acceptable salt form.

In some embodiments, the disease or disorder is a hyperproliferative disease (e.g., cancer), an autoimmune disease (such as Crohn's disease or rheumatoid arthritis), a neurological disease (such as amyotrophic lateral sclerosis (ALS) and fronto-temporal dementia (FTD), and in particular C9FTD/ALS), diabetes or prediabetes, or Francois-Neetens corneal fleck dystrophy. In some embodiments, the disease or disorder is a cancer, such as a cancer having a genotype or phenotype indicative of PIKFYVE overactivity or Sac underactivity. Cancer which may be amenable to treatment with a PIKFYVE inhibitor include, but are not limited to, non-Hodgkin lymphoma, multiple myeloma, melanoma, liver cancer, glioblastoma, multiple myeloma, prostate cancer and breast cancer. In particular embodiments, the cancer is castration-resistant prostate cancer. In some embodiments the disease or disorder is infection by an enveloped virus, such as a virus which gains cellular entry by endocytosis. Such viruses include, but are not limited to, Ebola, influenza A, vesicular stomatitis virus, Lassa fever virus, lymphocytic choriomeningitis virus, and coronaviruses (including MERS-CoV, SARS-CoV and SARS-CoV-2). Thus, in another embodiment, the present disclosure provides a method for treating or preventing a viral infection by an enveloped virus, such as Ebola, influenza A, vesicular stomatitis virus, Lassa fever virus, lymphocytic choriomeningitis virus, and coronaviruses (including MERS-CoV, SARS-CoV and SARS-CoV-2).

The PIKFYVE inhibitor compounds described herein for the treatment or prophylaxis of a disease or disorder according to the foregoing methods may be used as a sole therapeutic agent or may be used in combination with one or more other therapeutic agents useful for the treatment of said diseases or disorders.

Such other agents include inhibitors of other protein kinases or other proteins associated with cancer development (for example, serine-threonine kinases, tyrosine kinases, growth factor receptors), traditional cytotoxic anticancer agents (e.g., DNA alkylating agents, antimetabolites, anti-microtubule agents, topoisomerase inhibitors, and cytotoxic antibiotics), and monoclonal antibody therapies (e.g., pembrolizumab, rituximab, trastuzumab, alemtuzumab, cetuximab, panitumumab, bevacizumab, and ipilimumab).

Small molecule targeted therapies include inhibitors of such proteins as Bcr-Abl kinase, PDGFR, EGFR, VEGFR, RAF kinases, Ras-kinases, c-Kit, Src kinase, ephrin receptors, HER2/neu (ErbB2), proteasomes, estrogen receptors, JAK kinase, ALK, Bcl-2, PARP, PI3K, Braf, MEK, MAPK, CDK, HSP90, mTOR, inhibitors of checkpoint proteins (e.g., PD1, PDL1 and CTLA inhibitors), and modulators of the adaptive and innate immune system. Examples of these small molecule inhibitors include pembrolizumab, imatinib, gefitinib, erlotinib, sorafenib, sunitinib, dasatinib, lapatinib, nilotinib, bortezomib, tamoxifen, tofacitinib, crizotinib, obatoclax, navitoclax, gossypol, iniparib, olaparib, perifosine, apatinib, vemurafenib, dabrafenib, trametinib, CDK inhibitors, temsirolimus, everolimus, vemurafenib, and trametinib. Cytotoxic chemotherapeutic agents include cyclophosphamide, chlormethine, uramustine, melphalan, chlorambucil, ifosfamide, bendamustine, carmustine, lomustine, streptozotocin, busulfan, cisplatin, carboplatin, dicycloplatin, eptaplatin, lobaplatin, miriplatin, nedaplatin, oxaliplatin, picoplatin, satraplatin, triplatin, procarbazine, altretamine, dacarbazine, temozolomide, 5-fluorouracil, 6-mercaptopurine, thioguanine, capecitabine, azacytidine, decitabine, nelarabine, cladribine, clofarabine, cytarabine, floxuridine, fludarabine, gemcitabine, pentostatin, hydroxycarbamide, methotrexate, pemetrexed, daunorubicin, doxorubicin, epirubicin, idarubicin, actinomycin-D, bleomycin, mitomycin-C, mitoxantrone, vincristine, vinblastine, vinorelbine, vindesine, vinflunine, paclitaxel, docetaxel, etoposide, teniposide, irinotecan, topotecan, novobiocin, aclarubicin, pirarubicin, and aclarubicin. In some embodiments, particularly for the treatment of a cancer having activated MET or RAS signaling pathways, the compounds of the present disclosure are combined with compounds which inhibit MET activity or inhibit RAS activity, or inhibit upstream or downstream effectors in the MET or RAS signaling pathways, such as salirasib, tipifarnib, lonafarnib, crizotinib, cabozanitib, tivantinib, and tepotinib. In a particular embodiment, the cancer to eb treated is a prostate cancer (e.g., a castration-resistant prostate cancer and the compound of the present disclosure is combined with an anti-PD-1 antibody or a PD-1 inhibitor, such as pembrolizumab.

Such other agents also include small-molecule antiviral agents, such entry inhibitors, uncoating inhibitors, transcription or reverse transcription inhibitors, integrase inhibitors, translation inhibitors, protease inhibitors, assembly inhibitors, release inhibitors, and immune system stimulants (e.g., interferons). Examples of such agents include: abacavir, acyclovir, adefovir, amantadine, ampligen, amprenavir, arbidol umfenovir, atazanavir, atripla, baloxavir marboxil, biktarvy, boceprevir, bulevirtide, cidofovir, cobicistat, combivir, daclatasvir, darunavir, delavirdine, descovy, didanosine, docosanol, dolutegravir, doravirine, edoxudine, efavirenz, elvitegravir, emtricitabine, enfuvirtide, entecavir, etravirine, famciclovir, fomivirsen, fosamprenavir, foscarnet, ganciclovir, ibacitabine, ibalizumab, idoxuridine, imiquimod, imunovir, indinavir, lamivudine, letermovir, lopinavir, loviride, maraviroc, methisazone, moroxydine, nelfinavir, nevirapine, nexavir, nitazoxanide, norvir, oseltamivir, penciclovir, peramivir, pleconaril, raltegravir, remdesivir, ribavirin, rifampicin, rimantadine, ritonavir, saquinavir, simeprevir, sofosbuvir, stavudine, taribavirin, telaprevir, telbivudine, tenofovir, tenofovir alafenamide, tenofovir disoproxil, tipranavir, trifluridine, trizivir, tromantadine, Truvada, umifenovir, valaciclovir, valganciclovir, vicriviroc, vidarabine, zalcitabine, zanamivir, and zidovudine.

Other agents that might be combined with the compounds of the present invention include: corticosteroids, methotrexate, thiopurine, chloroquine, hydroxychloroquine, sulfasalazine, leflunomide, certolizumab, infliximab, etanercept, abatacept, anakinra, rituximab, tocilizumab, cyclosporin, golimumab, adalimumab, insulin, exenatide, liraglutide, pramlintide, metformin, phenformin, buformin, rosiglitazone, pioglitazone, troglitazone, tolbutamide, acetohexamide, tolazamide, chlorpropamide, glipizide, glyburide, gilmepiride, gliclazide, glyclopyramide, gliquidone, meglitinide, repaglinide, nateglinide, miglitol, acarbose, voglibose, taspoglutide, lixisenatide, semaglutide, dulaglutide, vildagliptin, sitagliptin, saxagliptin, linagliptin, alogliptin, septagliptin, teneligliptin, gemigliptin, dapagliflozin, canagliflozin, empagliflozin, and remogliflozin,

In another aspect, the invention provides the following:
(i) the compound of Formula I or any of 1.1-1.55 as described herein, in free or pharmaceutically acceptable salt form, for use in any of the methods or in the treatment or prophylaxis of any disease or disorder as set forth herein,
(ii) a combination as described hereinbefore, comprising a compound of Formula I or any of 1.1-1.55 as described herein, in free or pharmaceutically acceptable salt form and a second therapeutic agent useful for the treatment or prophylaxis of any disease or disorder set forth herein;
(iii) use of the compound of Formula I or any of 1.1-1.55, in free or pharmaceutically acceptable salt form, or the combination described herein, (in the manufacture of a medicament) for the treatment or prophylaxis of any disease or condition as set forth herein,
(iv) the compound of Formula I or any of 1.1-1.55, in free or pharmaceutically acceptable salt form, the combination described herein or the pharmaceutical composition of the invention as hereinbefore described for use in the treatment or prophylaxis of any disease or condition as set forth herein.

If not otherwise specified or clear from context, the following terms herein have the following meanings:
(a) “Alkyl” as used herein is a saturated or unsaturated hydrocarbon moiety, preferably saturated, preferably having one to six carbon atoms, in some embodiments, one to four carbon atoms, which may be linear or branched, and may be optionally mono-, di- or tri-substituted, e.g., with halogen (e.g., chloro or fluoro) or hydroxy. Exemplary “C₁₋₆ alkyl” groups include methyl, ethyl, n-propyl, isopropyl, n-butyl, isobutyl, tert-butyl, etc. Of course, other “C₁₋₆ alkyl” groups will be readily apparent to those of skill in the art given the benefit of the present disclosure. Alkyl substituents may be identified by commonly understood abbreviations, such as “Me” for methyl, “Et” for ethyl, “iPr” for isopropyl, and the like.
(b) “Aryl” as used herein means any carbocyclic aromatic ring system, i.e., any aromatic ring system comprising only carbon atoms as ring atoms. This includes 6-membered monocyclic aryl ring systems and 9-membered or 10-membered fused bicyclic aryl ring systems, and larger fused ring systems, as long such ring systems comprise at least one 6-membered aromatic carbocyclic ring (i.e., a benzene ring) within the fused ring system, and as long as no ring-atoms are heteroatoms. Aryl includes phenyl and napthyl.
(c) “Heteroaryl” as used herein means any cyclic heteroaromatic ring system, i.e., any aromatic ring system comprising at least one heteroatom (e.g., N, S, or O) ring atom. This includes 5-membered and 6-membered monocyclic heteroaryl ring systems and 9-membered or 10-membered fused bicyclic heteroaryl ring systems, and larger fused ring systems, as long such ring systems comprise at least one aromatic carbocyclic or aromatic heterocyclic ring within the fused ring system and at least one heteroatom (e.g., N, S or O) ring-atom within the fused ring system (either in an aromatic ring or non-aromatic ring). Heteroaryl therefore includes bicyclic fused ring system selected from aromatic-heteroaromatic, aromatic-heterocyclic, heteroaromatic-carbocyclic, heterocyclic-aromatic, and heteroaromatic-heteroaromatic, as well as larger fused ring systems comprising some combination of benzene, cycloalkane, heterocycloalkane and heteroaromatic rings. Exemplary heteroaryl groups include furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidinyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, 6,7-dihydro-5H-[1]pyridinyl, benzo[b]thiophenyl, 5,6,7,8-tetrahydro-quinolin-3-yl, benzoxazolyl, benzothiazolyl, benzisothiazolyl, benzisoxazolyl, benzimidazolyl, thianapthenyl, isothianapthenyl, benzofuranyl, isobenzofuranyl, isoindolyl, indolyl, indolizinyl, indazolyl, isoquinolyl, quinolyl, phthalazinyl, quinoxalinyl, quinazolinyl and benzoxazinyl, etc. It is understood that for heteroaryl systems in which the both ring carbon atoms and ring heteroatoms have open valencies, bonds can be formed to either such atom types (e.g., C-linked or N-linked). For example, where a pyrazolyl moiety is the group A, substituted at one atom to connect to the core of the compound of Formula I, and substituted at one or more other atoms with other substituent groups, either the core of the Compound of Formula I or any one or more other substituents may be attached to either a pyrazole ring nitrogen atom (N-linked) or a pyrazole ring carbon atom (C-linked).
(d) “Heterocycloalkyl” means any cyclic nonaromatic ring system comprising at least one heteroatom (e.g., N, S, or O) ring atom. This includes 3- to 12-membered monocyclic and fused bicyclic ring systems, and any larger multi-ring fused ring systems, as long such ring systems do not comprise any aromatic carbocyclic or aromatic heterocyclic ring. Exemplary heterocycloalkyl groups include pyrrolidinyl, tetrahydrofuranyl, dihydrofuranyl, tetrahydropyranyl, pyranyl, thiopyranyl, azindinyl, azetidinyl, oxiranyl, methylenedioxyl, chromenyl, barbituryl, isoxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, tetrahydroazepinyl, piperazinyl, piperizin-2-onyl, piperizin-3-onyl, chromanyl, 2-pyrrolinyl, 3-pyrrolinyl, imidazolidinyl, 2-imidazolidinyl, 1,4-dioxanyl, 8-azabicyclo[3.2.1]octanyl, 3-azabicyclo[3.2.1]octanyl, 3,8-diazabicyclo[3.2.1]octanyl, 2,5-diazabicyclo[2.2.1]heptanyl, 2,5-diazabicyclo[2.2.2]octanyl, octahydro-2H-pyrido[1,2-a]pyrazinyl, 3-azabicyclo[4.1.0]heptanyl, 3-azabicyclo[3.1.0]hexanyl, 2-azaspiro[4.4]nonanyl, 7-oxa-1-azaspiro[4.4]nonanyl, 7-azabicyclo[2.2.2]heptanyl, octahydro-1H-indolyl, etc. In general, the heterocycloalkyl group typically is attached to the main structure via a carbon atom or a nitrogen atom. Of course, other heterocycloalkyl groups will be readily apparent to those of skill in the art given the benefit of the present disclosure.
(e) “Cycloalkyl” means a nonaromatic saturated or unsaturated free radical forming at least one ring consisting essentially of 3 to 10 carbon atoms and a corresponding number of hydrogen atoms. The term “cycloalkyl” therefore includes cycloalkenyl groups, as further defined below. As such, cycloalkyl groups can be monocyclic or polycyclic. Individual rings of such polycyclic cycloalkyl groups can have different connectivities, e.g., fused, bridged, spiro, etc., in addition to covalent bond substitution. Exemplary cycloalkyl groups include cyclopropyl, cyclobutyl, cyclopentyl, cyclohexyl, norbornanyl, bicyclo[3.2.1]octanyl, octahydro-pentalenyl, spiro[4.5]decanyl, adamantyl, cyclopropyl substituted with cyclobutyl, cyclobutyl substituted with cyclopentyl, cyclohexyl substituted with cyclopropyl, etc. Of course, other cycloalkyl groups will be readily apparent to those of skill in the art given the benefit of the present disclosure.
(f) “Substituted” and “optionally substituted”, in the context of substituted variations of a saturated carbocyclic or heterocyclic ring (e.g., C₃₋₆cycloalkyl or 3- to 8-membered heterocycloalkyl) includes spiro substitution of the ring. For example, a cyclopropane ring spiro-joined to a cyclohexane ring is within the scope of an “optionally substituted C₃₋₆cycloalkyl” because the spiro cyclopropane is a substituent of the cyclohexane (C₆) ring, despite the complete ring system having 8 carbon atoms. Similarly, a piperidine ring with a spiro-joined morpholine ring is within the scope of an “optionally substituted 3-to-8-membered heterocycloalkyl” because the morpholine ring is a substituent of the piperidine ring, despite the complete spiro ring system having 11 members. Therefore, as used herein, the terms “substituted” and “optionally substituted”, in the context of substituted variations of a saturated carbocyclic or heterocyclic ring includes the option of substitution by one or more (e.g., 2 or 3) spiro-joined C₃₋₆cycloalkyl rings and one or more (e.g., 2 or 3) spiro joined 3-6 membered heterocycloalkyl rings, or a combination thereof. Thus, for examples, this includes morpholine with a spiro-joined oxetane, piperidine with a spiro-joined cyclopropane, cyclohexane with a spiro-joined cyclopentane, cyclopentane with a spiro-joined oxetane, etc. As used herein, such substitutions may be indicated by the term “spiro,” for example, “piperidine-spiro-oxetane.”

It is understood that when describing the substituents attached in various positions to the core structure of Formula I, including substituents attached to substituents, in some cases, the substituent may be referred to using the name of the corresponding chemical compound, especially in the case of rings, whereas in some cases the same substituent may be referred to using the name of the corresponding chemical radical (e.g., having an “-yl” suffix), but these terms are interchangeable. For example, when referring to the substituent B, or a heteroaryl ring attached to a substituent A, the terms “pyridine” and “pyridyl” are equivalent, as are the terms “morpholine” and “morpholinyl.” The skilled artisan will recognize that such terms are used to denote attachment of, for example, a pyridine or morpholine ring at the designated position, thus converting said ring to a pyridyl or morpholinyl substituent, respectively. Absent an indication otherwise, such attachments may be made at any chemically permissible location of the attached ring.

Compounds of the Invention, e.g., the compound of Formula I or any of formulae 1.1-1.55 as described herein, may exist in free or salt form, e.g., as acid addition salts (e.g., hydrochloride).

In this specification unless otherwise indicated, language such as “Compounds of the Invention” is to be understood as embracing the compounds in any form, for example free or acid addition salt form, or where the compounds contain acidic substituents, in base addition salt form. The Compounds of the Invention are intended for use as pharmaceuticals, therefore pharmaceutically acceptable salts are preferred. Salts which are unsuitable for pharmaceutical uses may be useful, for example, for the isolation or purification of free Compounds of the Invention or their pharmaceutically acceptable salts, are therefore also included.

The Compounds of the Invention include their enantiomers, diastereomers and racemates, as well as their polymorphs, hydrates, solvates and complexes. Some individual compounds within the scope of this invention may contain double bonds. Representations of double bonds in this invention are meant to include both the E and the Z isomer of the double bond. In addition, some compounds within the scope of this invention may contain one or more asymmetric centers. This invention includes the use of any of the optically pure stereoisomers as well as any combination of stereoisomers.

The Compounds of the present disclosure may comprise one or more chiral carbon atoms. The compounds thus exist in individual isomeric, e.g., enantiomeric or diastereomeric form or as mixtures of individual forms, e.g., racemic/diastereomeric mixtures. Any isomer may be present in which the asymmetric center is in the (R)-, (S)-, or (R,S)-configuration. The invention is to be understood as embracing both individual optically active isomers as well as mixtures (e.g., racemic/diastereomeric mixtures) thereof. Accordingly, the Compounds of the Invention may be a racemic mixture or it may be predominantly, e.g., in pure, or substantially pure, isomeric form, e.g., greater than 70% enantiomeric/diastereomeric excess (“e.e.”), preferably greater than 80% e.e., more preferably greater than 90% e.e., most preferably greater than 95% e.e. The purification of said isomers and the separation of said isomeric mixtures may be accomplished by standard techniques known in the art (e.g., column chromatography, preparative TLC, preparative HPLC, simulated moving bed and the like).

It is also intended that the Compounds of the Invention encompass their stable and unstable isotopes. Stable isotopes are nonradioactive isotopes which contain one additional neutron compared to the abundant nuclides of the same species (i.e., element).

It is expected that the activity of compounds comprising such isotopes would be retained, and such compound would also have utility for measuring pharmacokinetics of the non-isotopic analogs. For example, the hydrogen atom at a certain position on the Compounds of the Invention may be replaced with deuterium (a stable isotope which is non-radioactive). Examples of known stable isotopes include, but are not limited to, deuterium (²H), ¹³C, ¹⁵N, is ¹⁸O. Alternatively, unstable isotopes, which are radioactive isotopes which contain additional neutrons compared to the abundant nuclides of the same species (i.e., element), e.g., tritium (³H), ¹²³I, ¹³¹I, ¹²⁵I, ¹⁴C, ¹⁸F, may replace the corresponding abundant species of H, I, C and F. Another example of useful isotope of the compound of the invention is the ¹¹C isotope. These radio isotopes are useful for radio-imaging and/or pharmacokinetic studies of the compounds of the invention. Thus, for example, any recitation of a methyl group, also embraces a CD₃ group (e.g., a 3-methylphenyl substituent can be d₃-methylphenyl substituent).

Melting points are uncorrected and (dec) indicates decomposition. Temperatures are given in degrees Celsius (° C.); unless otherwise stated, operations are carried out at room or ambient temperature, that is, at a temperature in the range of 18-25° C. Chromatography means flash chromatography on silica gel; thin layer chromatography (TLC) is carried out on silica gel plates. NMR data is in the delta values of major diagnostic protons, given in parts per million (ppm) relative to tetramethylsilane (TMS) as an internal standard. Conventional abbreviations for signal shape are used. Coupling constants (J) are given in Hz. For mass spectra (MS), the lowest mass major ion is reported for molecules where isotope splitting results in multiple mass spectral peaks Solvent mixture compositions are given as volume percentages or volume ratios. In cases where the NMR spectra are complex, only diagnostic signals are reported.

The words “treatment” and “treating” are to be understood accordingly as embracing treatment or amelioration of symptoms of disease as well as treatment of the cause of the disease. For methods of treatment, the word “effective amount” is intended to encompass a therapeutically effective amount to treat a specific disease or disorder.

The term “patient” includes human and non-human (i.e., animal) patients. In particular embodiments, the invention encompasses both human and nonhuman patients. In another embodiment, the invention encompasses non-human patients. In other embodiment, the term encompasses human patients.

The term “comprising” as used in this disclosure is intended to be open-ended and does not exclude additional, unrecited elements or method steps.

Compounds of the Invention, e.g., compounds of Formula I or any of formulas 1.1-1.55 as hereinbefore described, in free or pharmaceutically acceptable salt form, may be used as a sole therapeutic agent, but may also be used in combination or for co-administration with other active agents.

Dosages employed in practicing the methods of present invention will of course vary depending, e.g., on the particular disease or condition to be treated, the particular compound used, the mode of administration, and the therapy desired. The compound may be administered by any suitable route, including orally, parenterally, transdermally, or by inhalation, but are preferably administered orally. In general, satisfactory results, e.g., for the treatment of diseases as hereinbefore set forth are indicated to be obtained on oral administration at dosages of the order from about 0.001 to 2.0 mg/kg. In larger mammals, for example humans, an indicated daily dosage for oral administration will accordingly be in the range of from about 0.01 to 1000 mg, conveniently administered once, or in divided doses 2 to 4 times, daily or in sustained release form. Unit dosage forms for oral administration thus for example may comprise from about 0.2 to 75 or 150 mg or 300 mg, e.g., from about 0.2 or 2.0 to 10, 25, 50, 75, 100, 150, 200 or 300 mg of the compound disclosed herein, together with a pharmaceutically acceptable diluent or carrier therefor.

The term “pharmaceutically acceptable diluent or carrier” is intended to mean diluents and carriers that are useful in pharmaceutical preparations, and that are free of substances that are allergenic, pyrogenic or pathogenic, and that are known to potentially cause or promote illness. Pharmaceutically acceptable diluents or carriers thus exclude bodily fluids such as example blood, urine, spinal fluid, saliva, and the like, as well as their constituent components such as blood cells and circulating proteins. Suitable pharmaceutically acceptable diluents and carriers can be found in any of several well-known treatises on pharmaceutical formulations, for example Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000; and Martindale, The Extra Pharmacopoeia, Thirty-Second Edition (The Pharmaceutical Press, London, 1999); all of which are incorporated by reference herein in their entirety.

All numerical designations, e.g., pH, temperature, time, concentration, molecular weight, including ranges, are approximations which are varied (+) or (−) by increments of 0.1 or 1.0, where appropriate. It is to be understood, although not always explicitly stated, that all numerical designations are preceded by the term “about.” Depending on the context, the exact bounds of the term “about” will vary but will be understandable to those of skill in the art. In some contexts, for example, the term “about” may indicate a deviation from a stated value or range of +/−10%, or +/−25%, or +/−50%. It also is to be understood, although not always explicitly stated, that the reagents described herein are merely exemplary and that equivalents of such are known in the art.

Pharmaceutical compositions comprising Compounds of the Invention may be prepared using conventional diluents or excipients and techniques known in the galenic art. Thus, oral dosage forms may include tablets, capsules, solutions, suspensions and the like.

### Methods of Making Compounds of the Invention

The Compounds of the Invention and their pharmaceutically acceptable salts may be made using the methods as described and exemplified herein and/or by methods similar thereto and/or by methods known in the chemical art. Such methods include, but not limited to, those described below. If not commercially available, starting materials for these processes may be made by procedures, which are selected from the chemical art using techniques which are similar or analogous to the synthesis of known compounds.

For example, Compounds of the Invention may be prepared according to the general synthetic scheme provided below, and as shown in the Examples section below.

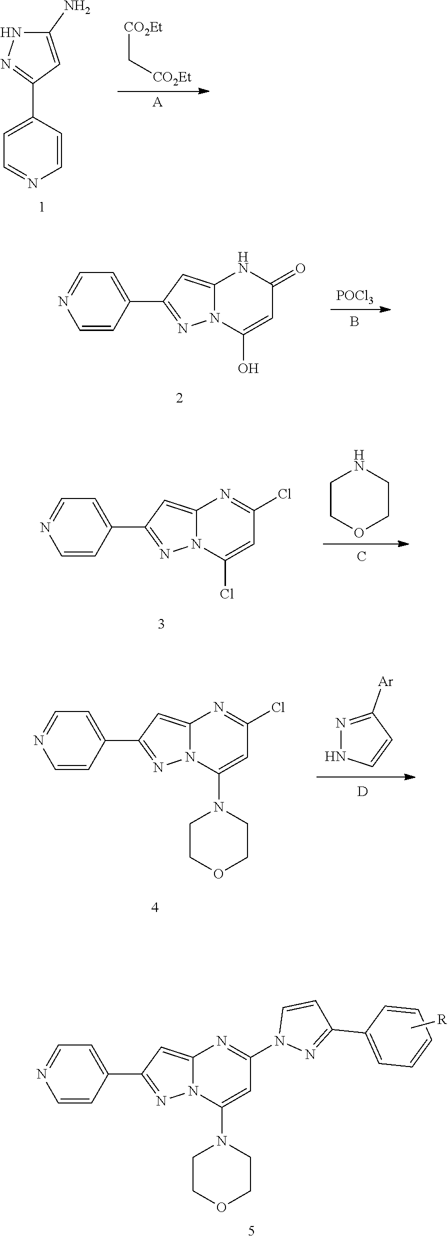

### Terms and Abbreviations

AcN: Acetonitrile
m-CPBA: meta-chloroperoxybenzoic acid
DBU: 1,8-Diazabicyclo[5.4.0]undec-7-ene
DCM: Dichloromethane
DIPEA: N,N-diisopropylethylamine
DMF: Dimethylformamide
DMSO: Dimethylsulfoxide
DMTMM: 4-(4,6-Dimethoxy[1,3,5]triazin-2-yl)-4-methylmorpholinium chloride
EtOAc: Ethyl acetate
HATU: 1-[Bis(dimethylamino)methylene]-1H-1,2,3-triazolo[4,5-b]pyridinium 3-oxide hexafluorophosphate
HCl: hydrochloric acid
H₂O: Water
HPLC: High-pressure liquid chromatography
LCMS: Liquid chromatography-mass spectroscopy
MeOH: Methanol
MeCN: Acetonitrile
NaH: sodium hydride
NaOH: Sodium Hydroxide
NMR: nuclear magnetic resonance spectroscopy
POCl₃: Phosphorus oxychloride
RT: Room temperature
SOCl₂: Thionyl chloride
t-butylXphos: 2-Di-tert-butylphosphino-2′,4′,6′-triisopropylbiphenyl
TBSCl: tert-Butyldimethylsilyl chloride
TBAF: Tetra-n-butylammonium fluoride
TEA: Triethylamine
T3P: Propylphosphonic anhydride
THF: Tetrahydrofuran
TLC: Thin-layer chromatography

### EXAMPLES

### Example 1

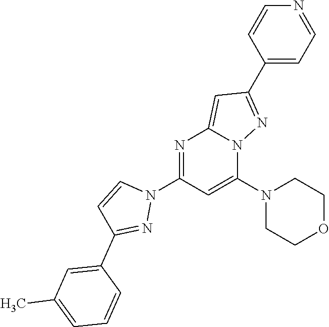 [SMILES: Cc1cccc(-c2ccn(-c3cc(N4CCOCC4)n4nc(-c5ccncc5)cc4n3)n2)c1]

Step A: To a solution of compound 1 (15 g, 93.6 mmol) in methanol (250 mL) were added sodium methylate (5 g, 93.6 mmol) and diethyl malonate (14.2 mL, 93.6 mmol) and the mixture was stirred under reflux for 9 hours. After cooling the reaction mixture, the obtained precipitate was filtered off and dried at 80° C. to give compound 2 (10 g, 44 mmol, 47% yield).

Step B: To compound 2 (10 g, 44 mmol) was added phosphoryl chloride (100 mL) and the resulting suspension was stirred for 4 hours at 110° C. After that, phosphoryl chloride was distilled off from the reaction mixture, ethanol was added to the residue with ice-cooling and the mixture was stirred for 15 minutes and concentrated in vacuo. The residue was purified by silica gel column chromatography (methanol/chloroform) to give compound 3 (3 g, 11.32 mmol, 26%).

Step C: A solution of 5,7-dichloro-4-yl-2-pyridin-4-yl-pyrazolo[1,5-a]pyrimidine 3 (1.5 g, 5.64 mmol) in 1,4-dioxane (50 mL) was prepared and morpholine (1 mL, 11.32 mmol) was added to the solution. The mixture was stirred at room temperature for 16 h. After completion of the reaction, the suspension was concentrated to dryness and the residue was purified by flash column chromatography (methanol/chloroform) to give compound (4) (1.1 g, 3.64 mmol, 65%).

Step D: To a stirred suspension of NaH (0.007 g, 0.174 mmol, 60% dispersion in mineral oil) in DMSO (1 mL) was added 3-(m-tolyl)-1H-pyrazole (0.027 g, 0.174 mmol) and the reaction mixture was stirred at r.t. for 30 min. Then 4-(5-chloro-2-(pyridin-4-yl)pyrazolo[1,5-a]pyrimidin-7-yl)morpholine (1) (0.050 g, 0.158 mmol) was added and the reaction mixture was stirred at 110° C. overnight, cooled to r.t. and diluted with water (5 mL). The solid was filtered and washed with warm hexanes to give desired product (31 mg, 45%). LCMS (ESI) m/z 438 [M+1]⁺ (100% purity, RT 1.53 min).

### Example 2

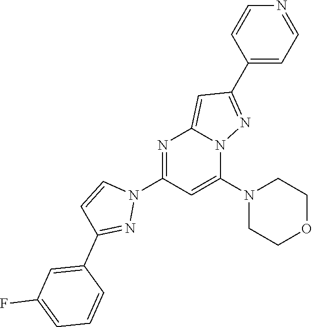 [SMILES: Fc1cccc(-c2ccn(-c3cc(N4CCOCC4)n4nc(-c5ccncc5)cc4n3)n2)c1]

Step D: To a stirred suspension of NaH (0.007 g, 0.174 mmol, 60% dispersion in mineral oil) in DMSO (1 mL) was added 3-(3-fluorophenyl)-1H-pyrazole (0.028 g, 0.174 mmol) and the reaction mixture was stirred at r.t. for 30 min. Then 4-(5-chloro-2-(pyridin-4-yl)pyrazolo[1,5-a]pyrimidin-7-yl)morpholine (1) (0.050 g, 0.158 mmol) was added and the reaction mixture was stirred at 110° C. overnight, cooled to r.t. and diluted with water (5 mL). The solid was filtered and washed with warm hexanes to give desired product (29 mg, 42%). LCMS (ESI) m/z 442 [M+1]⁺ (100% purity, RT 1.49 min).

### Example 3

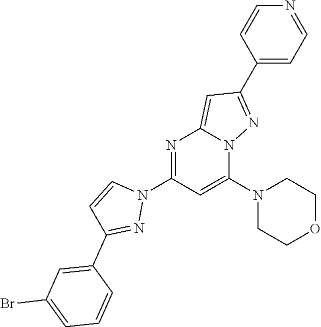 [SMILES: Brc1cccc(-c2ccn(-c3cc(N4CCOCC4)n4nc(-c5ccncc5)cc4n3)n2)c1]

Step D: To a stirred suspension of NaH (0.007 g, 0.174 mmol, 60% dispersion in mineral oil) in DMSO (1 mL) was added 3-(3-bromophenyl)-1H-pyrazole (0.039 g, 0.174 mmol) and the reaction mixture was stirred at r.t. for 30 min. Then 4-(5-chloro-2-(pyridin-4-yl)pyrazolo[1,5-a]pyrimidin-7-yl)morpholine (1) (0.050 g, 0.158 mmol) was added and the reaction mixture was stirred at 110° C. overnight, cooled to r.t. and diluted with water (5 mL). The solid was filtered and washed with warm hexanes to give desired product (20 mg, 25%). LCMS (ESI) m/z 503 [M+1]⁺ (100% purity, RT 1.44 min).

### Example 4

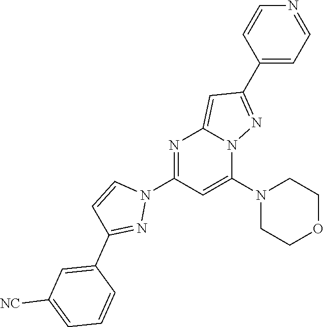 [SMILES: N#Cc1cccc(-c2ccn(-c3cc(N4CCOCC4)n4nc(-c5ccncc5)cc4n3)n2)c1]

Step D: To a stirred suspension of NaH (0.007 g, 0.174 mmol, 60% dispersion in mineral oil) in DMSO (1 mL) was added 3-(1H-pyrazol-3-yl)benzonitrile (0.029 g, 0.174 mmol) and the reaction mixture was stirred at r.t. for 30 min. Then 4-(5-chloro-2-(pyridin-4-yl)pyrazolo[1,5-a]pyrimidin-7-yl)morpholine (1) (0.050 g, 0.158 mmol) was added and the reaction mixture was stirred at 110° C. overnight, cooled to r.t. and diluted with water (5 mL). The solid was filtered and washed with warm hexanes to give crude compound (˜90% purity). The solid was washed with methanol to give desired product (21 mg, 30%). LCMS (ESI) m/z 449 [M+1]⁺ 100% purity, RT 1.37 min).

### Example 5

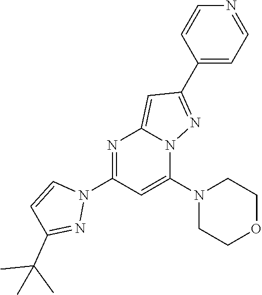 [SMILES: CC(C)(C)c1ccn(-c2cc(N3CCOCC3)n3nc(-c4ccncc4)cc3n2)n1]

Step D: To a stirred suspension of NaH (0.007 g, 0.174 mmol, 60% dispersion in mineral oil) in DMSO (1 mL) was added 3-(tert-butyl)-1H-pyrazole (0.022 g, 0.174 mmol) and the reaction mixture was stirred at r.t. for 30 min. Then 4-(5-chloro-2-(pyridin-4-yl)pyrazolo[1,5-a]pyrimidin-7-yl)morpholine (1) (0.050 g, 0.158 mmol) was added and the reaction mixture was stirred at 110° C. overnight, cooled to r.t. and diluted with water (5 mL). The solid was filtered and washed with warm hexanes to give desired product (15 mg, 24%). LCMS (ESI) m/z 404 [M+1]⁺ (100% purity, RT 1.48 min).

### Example 6

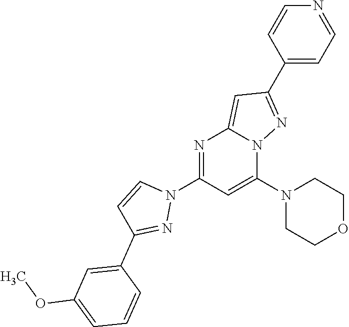 [SMILES: COc1cccc(-c2ccn(-c3cc(N4CCOCC4)n4nc(-c5ccncc5)cc4n3)n2)c1]

Step D: To a stirred suspension of NaH (0.007 g, 0.174 mmol, 60% dispersion in mineral oil) in DMSO (1 mL) was added 3-(3-methoxyphenyl)-1H-pyrazole (0.174 mmol) and the reaction mixture was stirred at r.t. for 30 min. Then 4-(5-chloro-2-(pyridin-4-yl)pyrazolo[1,5-a]pyrimidin-7-yl)morpholine (1) (0.050 g, 0.158 mmol) was added and the reaction mixture was stirred at 110° C. overnight, cooled to r.t. and diluted with water (5 mL). The solid was filtered and washed with methanol (1 mL) to give desired product (23 mg, 32%). LCMS (ESI) m/z 454 [M+1]⁺ (98% purity, RT 1.29 min).

### Example 7

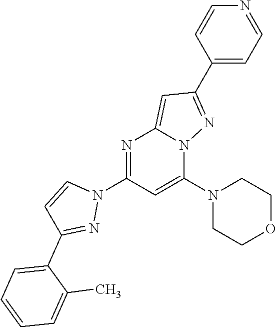 [SMILES: Cc1ccccc1-c1ccn(-c2cc(N3CCOCC3)n3nc(-c4ccncc4)cc3n2)n1]

Step D: To a stirred suspension of NaH (0.007 g, 0.174 mmol, 60% dispersion in mineral oil) in DMSO (1 mL) was added 3-(o-tolyl)-1H-pyrazole (0.174 mmol) and the reaction mixture was stirred at r.t. for 30 min. Then 4-(5-chloro-2-(pyridin-4-yl)pyrazolo[1,5-a]pyrimidin-7-yl)morpholine (1) (0.050 g, 0.158 mmol) was added and the reaction mixture was stirred at 110° C. overnight, cooled to r.t. and diluted with water (5 mL). The solid was filtered and purified by preparative HPLC to give desired product (14 mg, 20%). LCMS (ESI) m/z 448 [M+1]⁺ (100% purity, RT 3.48 min).

### Example 8

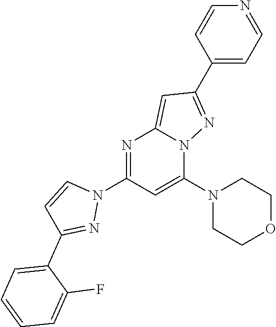 [SMILES: Fc1ccccc1-c1ccn(-c2cc(N3CCOCC3)n3nc(-c4ccncc4)cc3n2)n1]

Step D: To a stirred suspension of NaH (0.007 g, 0.174 mmol, 60% dispersion in mineral oil) in DMSO (1 mL) was added 3-(2-fluorophenyl)-1H-pyrazole (0.174 mmol) and the reaction mixture was stirred at r.t. for 30 min. Then 4-(5-chloro-2-(pyridin-4-yl)pyrazolo[1,5-a]pyrimidin-7-yl)morpholine (1) (0.050 g, 0.158 mmol) was added and the reaction mixture was stirred at 110° C. overnight, cooled to r.t. and diluted with water (5 mL). The solid was filtered and washed with methanol (1 mL) to give desired product (22 mg, 32%). LCMS (ESI) m/z 442 [M+1]⁺ (100% purity, RT 1.28 min).

### Example 9

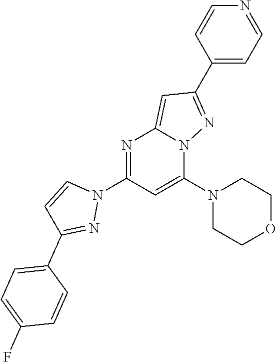 [SMILES: Fc1ccc(-c2ccn(-c3cc(N4CCOCC4)n4nc(-c5ccncc5)cc4n3)n2)cc1]

Step D: To a stirred suspension of NaH (0.007 g, 0.174 mmol, 60% dispersion in mineral oil) in DMSO (1 mL) was added 3-(4-fluorophenyl)-1H-pyrazole (0.174 mmol) and the reaction mixture was stirred at r.t. for 30 min. Then 4-(5-chloro-2-(pyridin-4-yl)pyrazolo[1,5-a]pyrimidin-7-yl)morpholine (1) (0.050 g, 0.158 mmol) was added and the reaction mixture was stirred at 110° C. overnight, cooled to r.t. and diluted with water (5 mL). The solid was filtered and washed with methanol (1 mL) to give desired product (41 mg, 59%). LCMS (ESI) m/z 442 [M+1]⁺ (100% purity, RT 1.30 min).

### Example 10

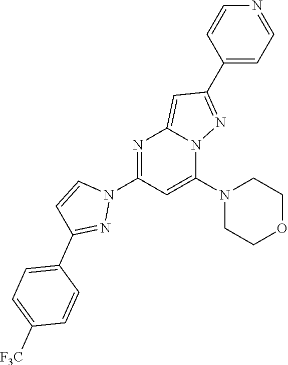 [SMILES: FC(F)(F)c1ccc(-c2ccn(-c3cc(N4CCOCC4)n4nc(-c5ccncc5)cc4n3)n2)cc1]

Step D: To a stirred suspension of NaH (0.007 g, 0.174 mmol, 60% dispersion in mineral oil) in DMSO (1 mL) was added 3-(4-trifluoromethyl)-1H-pyrazole (0.174 mmol) and the reaction mixture was stirred at r.t. for 30 min. Then 4-(5-chloro-2-(pyridin-4-yl)pyrazolo[1,5-a]pyrimidin-7-yl)morpholine (1) (0.050 g, 0.158 mmol) was added and the reaction mixture was stirred at 110° C. overnight, cooled to r.t. and diluted with water (5 mL). The solid was filtered and washed with methanol (1 mL) to give desired product (40 mg, 52%). LCMS (ESI) m/z 492 [M+1]⁺ (100% purity, RT 1.40 min).

### Examples 11-14

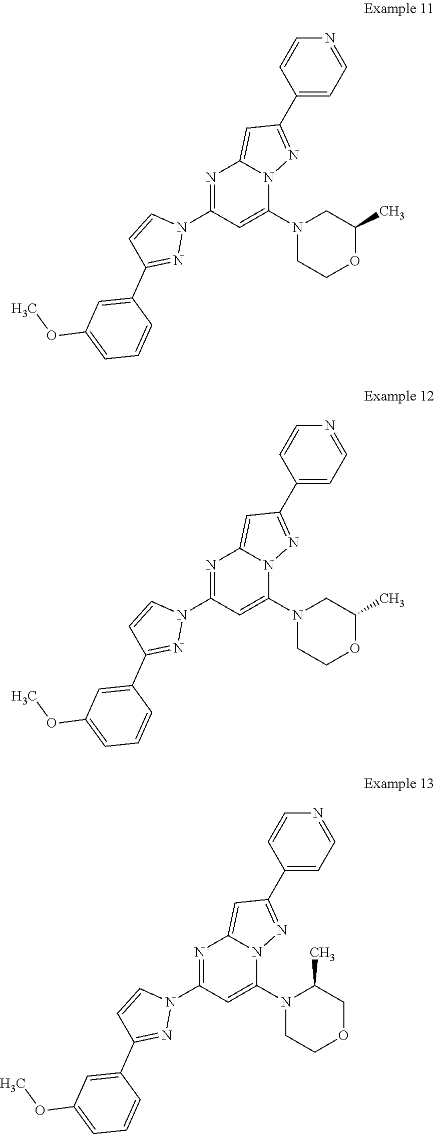

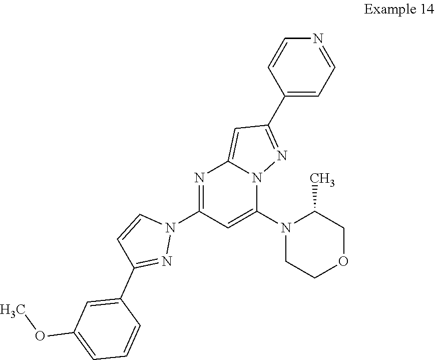 [SMILES: COc1cccc(-c2ccn(-c3cc(N4CCOC[C@H]4C)n4nc(-c5ccncc5)cc4n3)n2)c1]

The methyl-substituted morpholine compounds of Examples 11-14 may be prepared according to the following synthetic scheme:

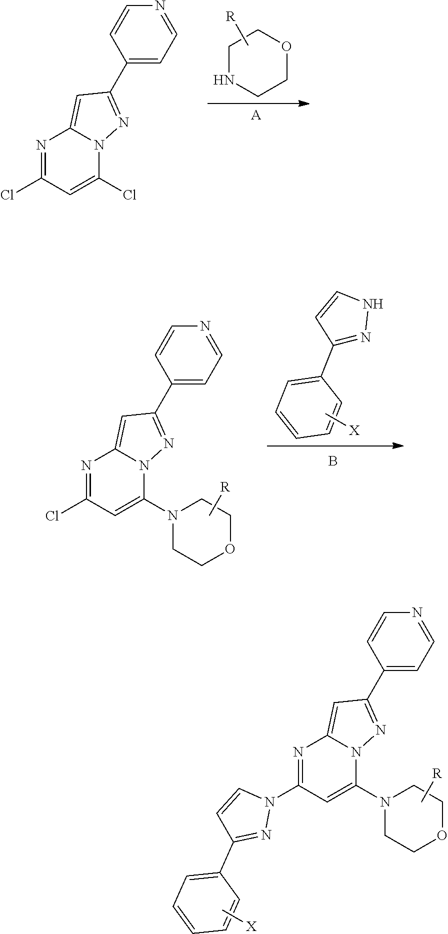

In the above scheme, 5,7-dichloro-2-(pyridin-4-yl)pyrazolo[1,5-a]pyrimidine is reacted in a nucleophilic displacement reaction with the appropriate enantiopure substituted morpholine (R is methyl). The resulting product is reacted with the appropriate substituted 3-phenylpyrazole (e.g., 3-(m-tolyl)-1H-pyrazole). The final product is isolated as similarly provided in the preceding examples.

### Examples 15-17

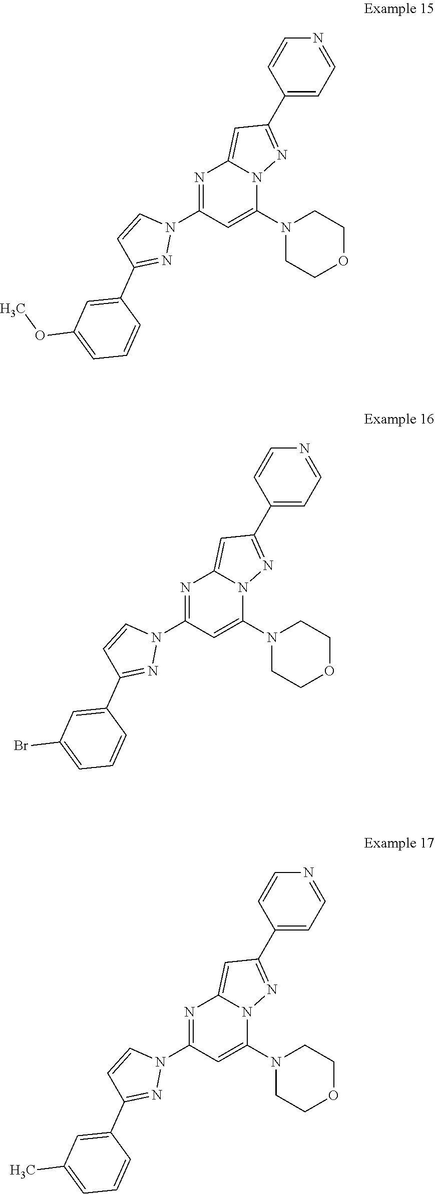 [SMILES: COc1cccc(-c2ccn(-c3cc(N4CCOCC4)n4nc(-c5ccncc5)cc4n3)n2)c1 ; Brc1cccc(-c2ccn(-c3cc(N4CCOCC4)n4nc(-c5ccncc5)cc4n3)n2)c1 ; Cc1cccc(-c2ccn(-c3cc(N4CCOCC4)n4nc(-c5ccncc5)cc4n3)n2)c1]

The 1-substituted-3-pyrazolyl compounds of Examples 15-17 may be prepared according to the following synthetic scheme:

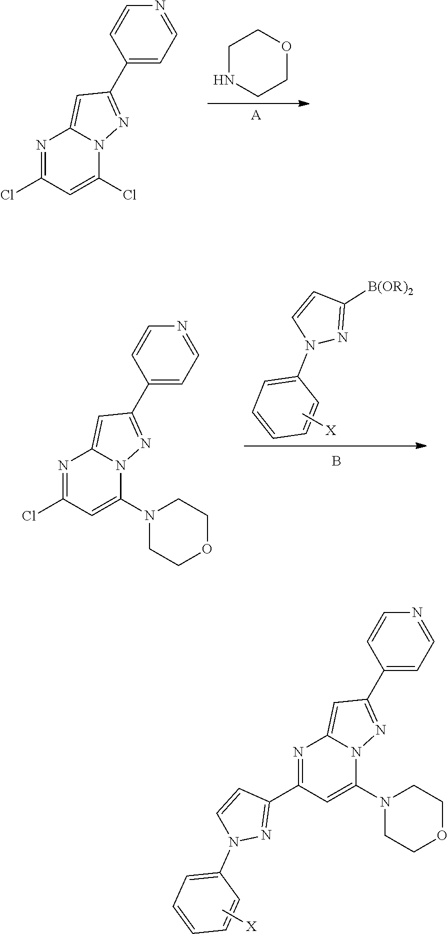

In the above scheme, 5,7-dichloro-2-(pyridin-4-yl)pyrazolo[1,5-a]pyrimidine is reacted in a nucleophilic displacement reaction with morpholine. The resulting product is reacted with the appropriate substituted 1-phenyl-3-pyrazolylboronic acid in a Suzuki coupling reaction. The final product is isolated as similarly provided in the preceding examples.

### Examples 18-29

Using procedures analogs to those described hereinabove, the following additional compounds are prepared:

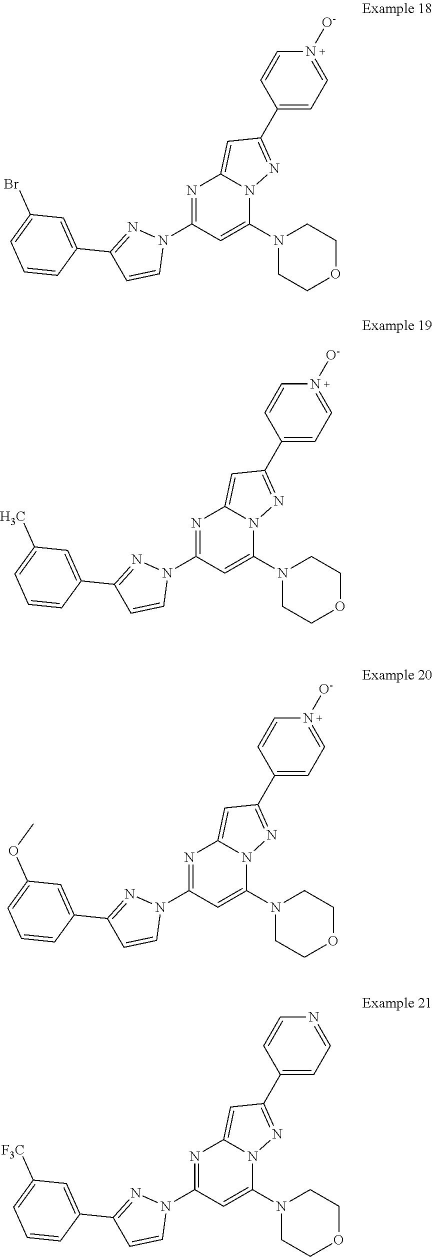

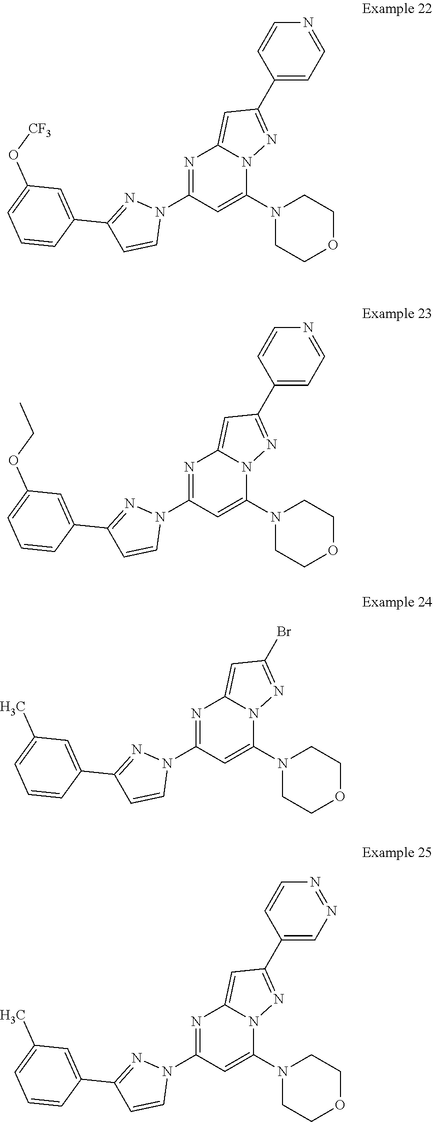 [SMILES: FC(F)(F)Oc1cccc(-c2ccn(-c3cc(N4CCOCC4)n4nc(-c5ccncc5)cc4n3)n2)c1 ; CCOc1cccc(-c2ccn(-c3cc(N4CCOCC4)n4nc(-c5ccncc5)cc4n3)n2)c1 ; Cc1cccc(-c2ccn(-c3cc(N4CCOCC4)n4nc(Br)cc4n3)n2)c1 ; Cc1cccc(-c2ccn(-c3cc(N4CCOCC4)n4nc(-c5ccnnc5)cc4n3)n2)c1]

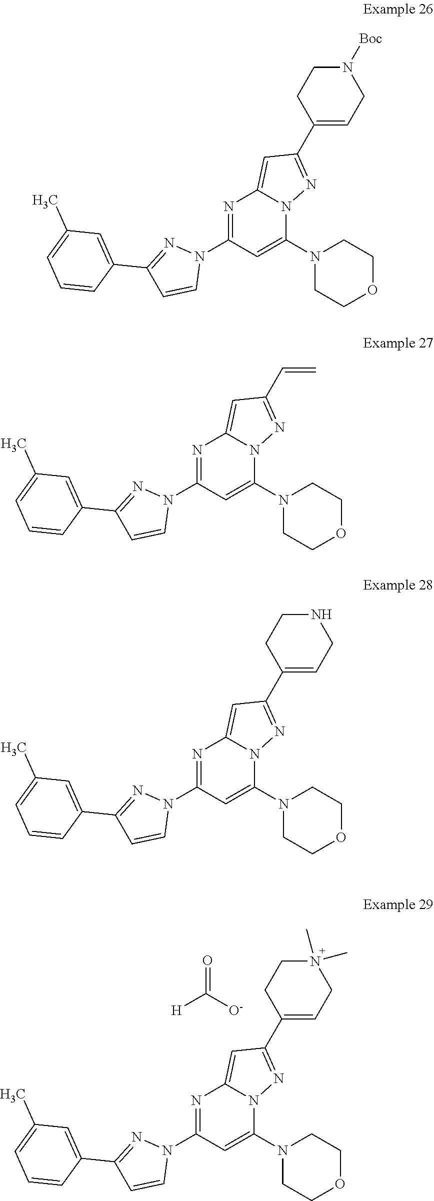

### Examples 18-20

The N-oxides of Examples 18-20 may be prepared according to the following synthetic scheme:

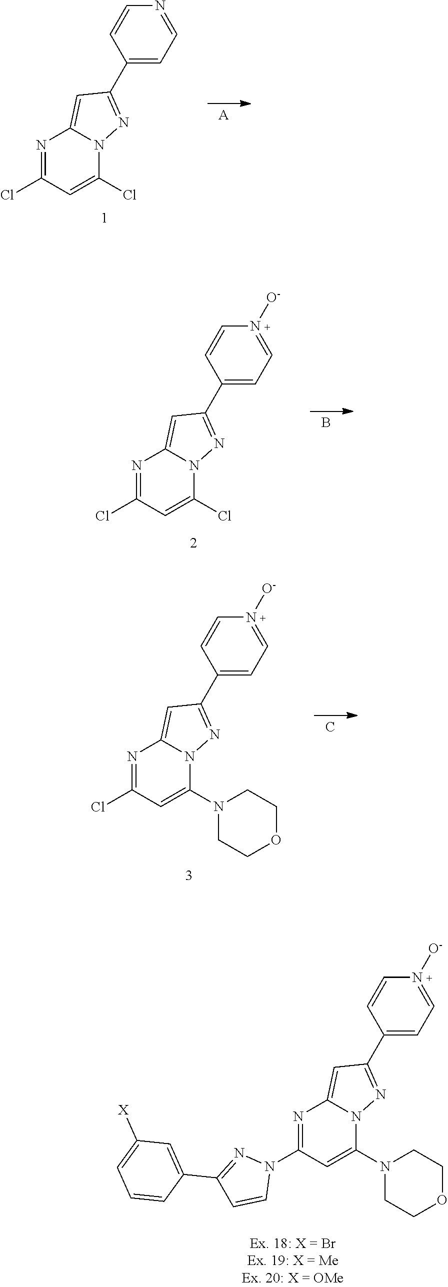

### Step A: 4-(5,7-dichloropyrazolo[1,5-a]pyrimidin-2-yl)pyridine 1-oxide (2)

To a stirred mixture of 5,7-dichloro-2-(pyridin-4-yl)pyrazolo[1,5-a]pyrimidine (1) (200 mg, 0.757 mmol, 1 eq.) in DCM (10 ml) at 0° C. was added m-CPBA (261 mg, 1.514 mmol, 2 eq.) and the reaction was allowed to stir at room temperature for 4 h. The reaction was monitored by TLC. After completion, the reaction mixture was directly purified via neutral alumina oxide column chromatography using 2-4% methanol in dichloromethane as an eluent to give the title compound (2) as a white solid (160 mg, 75.4% yield). Mass (m/z): 280.9 [M+H]⁺. LCMS purity: 69.84%.

### Step B: Synthesis of 4-(5-chloro-7-morpholinopyrazolo[1,5-a]pyrimidin-2-yl)pyridine 1-oxide (3)

To a stirred solution of 4-(5,7-dichloropyrazolo[1,5-a]pyrimidin-2-yl)pyridine 1-oxide (2) (150 mg, 0.535 mmol, 1 eq.) in 1,4-dioxane (12 ml) at 0° C. was added morpholine (118 mg, 0.642 mmol, 1.2 eq.) and the reaction mixture was stirred at room temperature for 16 h. After completion, the reaction mixture was evaporated under reduced pressure to obtain a crude product as an off-white solid. The crude product was further purified via silica gel (60-120 mesh) column chromatography using 3-5% methanol in dichloromethane as an eluent to give the title compound (3) as a white solid (150 mg, 84.7% yield). Mass (m/z): 332.0 [M+H]⁺. LCMS purity: 97.5%. ¹H-NMR (400 MHz, DMSO-ds): 8.32 (dd, J=5.2, 2.0 Hz, 2H), 8.01 (dd, J=5.2, 2.0 Hz, 2H), 7.18 (s, 1H), 6.50 (s, 1H), 3.90-3.88 (m, 4H), 3.84-3.82 (m, 4H)

### Step C: 4-(5-(3-(3-bromophenyl)-1H-pyrazol-1-yl)-7-morpholinopyrazolo[1,5-a]pyrimidin-2-yl)pyridine 1-oxide (Example 18)

To a stirred suspension of NaH (57-63%) (18 mg, 0.453 mmol, 3 eq.) in N-methyl-2-pyrrolidone (NMP) (6 mL) was added 3-(3-bromophenyl)-1H-pyrazole (101 mg, 0.453 mmol, 3 eq.) at 0° C. under N₂ atmosphere. The reaction mixture was stirred for 30 min at the same temperature. 4-(5-chloro-7-morpholinopyrazolo[1,5-a]pyrimidin-2-yl)pyridine 1-oxide (3) (50 mg, 0.151 mmol, 1 eq.) was added and the reaction was allowed to stir at 90° C. for 2 h in a sealed tube. After completion, the reaction was quenched with ice-cold water (20 mL) and extracted with ethyl acetate (3×20 mL). The combined organic layer was washed with brine (50 mL), dried over anhydrous sodium sulfate and evaporated under reduced pressure to obtain the crude material, which was purified by preparative HPLC to give the title compound Example 18 as an off-white solid (6 mg, 7.6% yield). Mass (m/z): 517.9 [M+H]⁺. LCMS purity: 96.34%. ¹H-NMR (400 MHz, DMSO-d₆): 8.75 (d, J=2.8 Hz, 1H), 8.33 (dd, J=5.6, 2.0 Hz, 2H), 8.21 (t, J=1.6 Hz, 1H), 8.05-8.02 (m, 3H), 7.63-7.60 (m, 1H), 7.47 (t, J=8 Hz, 1H), 7.26 (d, J=2.8 Hz, 1H), 7.18 (s, 1H), 7.04 (s, 1H), 3.93-3.92 (m, 8H)

### Step C: 4-(7-morpholino-5-(3-(m-tolyl)-1H-pyrazol-1-yl)pyrazolo[1,5-a]pyrimidin-2-yl)pyridine 1-oxide (Example 19)

To a stirred suspension of NaH (57-63%) (13 mg, 0.302 mmol, 2 eq.) in THF (5 mL) was added 3-(m-tolyl)-1H-pyrazole (24 mg, 0.151 mmol, 3 eq.) at 0° C. under N₂ atmosphere. The reaction mixture was stirred for 30 min at the same temperature. 4-(5-chloro-7-morpholinopyrazolo[1,5-a]pyrimidin-2-yl)pyridine 1-oxide (3) (50 mg, 0.151 mmol, 1 eq.) was added and the reaction was allowed to stir at 120° C. for 48 h in a sealed tube. After completion, the reaction mixture was quenched with ice-cold water (20 mL) and extracted with ethyl acetate (3×20 mL). The combined organic layer was washed brine (50 mL), dried over anhydrous sodium sulfate and evaporated under reduced pressure to obtain the crude material, which was purified by preparative HPLC to give the title compound Example 19 as an off-white solid (4 mg, 5.8% yield). Mass (m/z): 454.1 [M+H]⁺. LCMS purity: 98.81%. ¹H-NMR (400 MHz, DMSO-d₆): 8.72 (d, J=2.4 Hz, 1H), 8.33 (d, J=7.2 Hz, 2H), 8.03 (d, J=7.2 Hz, 2H), 7.83-7.80 (m, 2H), 7.38 (d, J=7.6 Hz, 1H), 7.25-7.22 (m, 1H), 7.17 (s, 1H), 7.15 (d, J=2.4 Hz, 1H), 7.03 (s, 1H), 3.92 (bs, 8H), 2.40 (s, 3H)

### Step C: 4-(5-(3-(3-methoxyphenyl)-1H-pyrazol-1-yl)-7-morpholinopyrazolo[1,5-a]pyrimidin-2-yl)pyridine 1-oxide (Example 20)

To a stirred suspension of NaH (57-63%) (15 mg, 0.362 mmol, 3 eq.) in N-methyl-2-pyrrolidone (NMP) (5 mL) was added 3-(3-methoxyphenyl)-1H-pyrazole (63 mg, 0.362 mmol, 3 eq.) at 0° C. under N₂ atmosphere. The reaction mixture was stirred for 30 min at the same temperature, and then 4-(5-chloro-7-morpholinopyrazolo[1,5-a]pyrimidin-2-yl)pyridine 1-oxide (3) (40 mg, 0.120 mmol, 1 eq.) was added, and the reaction was allowed to stir at 90° C. for 2 h in a sealed tube. After completion, the reaction mixture was quenched with ice-cold water (0.2 mL) and MeOH (0.2 mL), and evaporated under vacuum to obtain a crude product, which was purified via neutral alumina oxide column chromatography using 1-2% methanol in dichloromethane as an eluent to give the title compound Example 20 as a white solid (15 mg, 26.7% yield). Mass (m/z): 470.1 [M+H]⁺. LCMS purity: 96.23%. ¹H-NMR (400 MHz, DMSO-d₆): 8.73 (d, J=2.8 Hz, 1H), 8.33 (d, J=6.8 Hz, 2H), 8.03 (d, J=6.8 Hz, 2H), 7.60 (d, J=8 Hz, 1H), 7.54 (bs, 1H), 7.42 (t, J=7.6 Hz, 1H), 7.19-7.17 (m, 2H), 7.03 (s, 1H), 7.01-6.99 (m, 1H), 3.92 (bs, 8H), 3.85 (s, 3H)

### Examples 21-23

The compounds of Examples 21 to 23 may be prepared according to the following synthetic scheme:

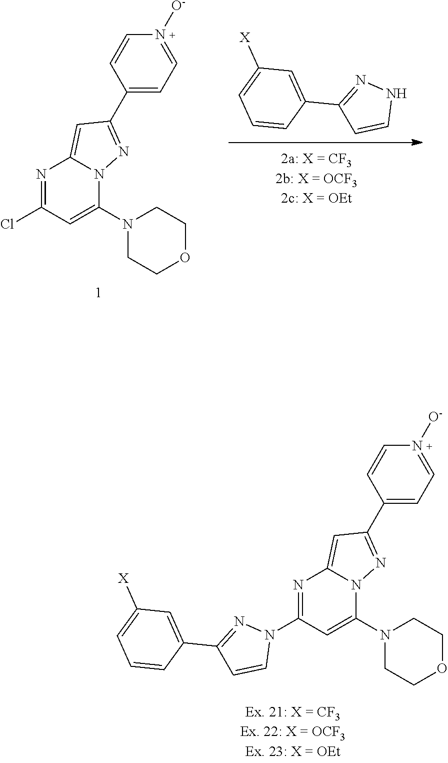

### General Procedure:

To a stirred suspension of NaH (0.007 g, 0.174 mmol, 60% dispersion in mineral oil) in DMSO (1 mL) was added corresponding the pyrazole (2a-c) (0.174 mmol) and the mixture was stirred at room temperature for 30 min. 4-(5-chloro-2-(pyridin-4-yl)pyrazolo[1,5-a]pyrimidin-7-yl)-morpholine (2) (0.050 g, 0.158 mmol) was added and the reaction mixture was stirred at 110° C. overnight, cooled to room temperature, and then diluted with water (5 mL). The resulting solid was filtered and washed with warm hexanes (5 mL) and methanol (2 mL) to give the desired product (Examples 21-23).

### 4-(2-(pyridin-4-yl)-5-(3-(3-(trifluoromethyl)phenyl)-1H-pyrazol-1-yl)pyrazolo[1,5-a]pyrimidin-7-yl)morpholine (Example 19)

Following the above general procedure, 61 mg of product was obtained (80% yield). Mass (m/z): 492.2 [M+H]⁺. LCMS purity: 100%.

### 4-(2-(pyridin-4-yl)-5-(3-(3-(trifluoromethoxy)phenyl)-1H-pyrazol-1-yl)pyrazolo[1,5-a]pyrimidin-7-yl)morpholine (Example 20)

Following the above general procedure, 63 mg of product was obtained (79% yield). Mass (m/z): 508.2 [M+H]⁺. LCMS purity: 100%.

### 4-(5-(3-(3-ethoxyphenyl)-1H-pyrazol-1-yl)-2-(pyridin-4-yl)pyrazolo[1,5-a]pyrimidin-7-yl)morpholine (Example 21)

Following the above general procedure, 38 mg of product was obtained (51% yield). Mass (m/z): 468.2 [M+H]⁺. LCMS purity: 100%.

### Example 24

The compound of Example 24 may be prepared according to the following synthetic scheme:

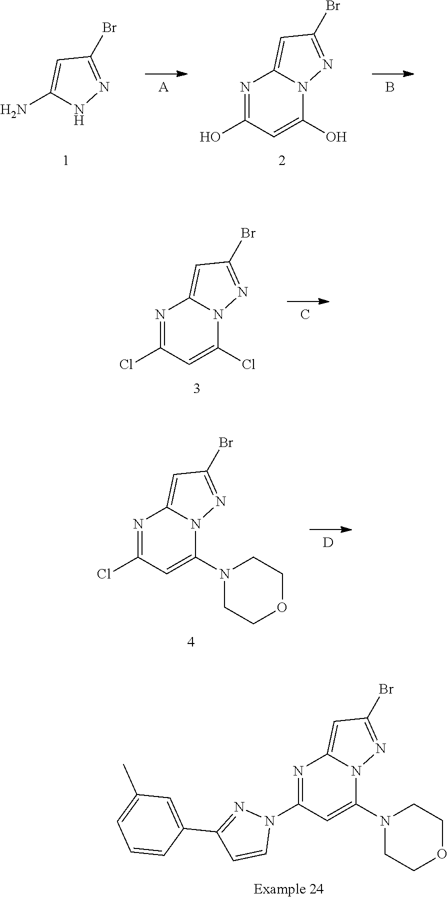

### Step A

To a solution of compound 1 (10 g, 61.7 mmol) in THF (200 mL) was added bis(2,4,6-trichlorophenyl)malonate (28.58 g, 61.7 mmol). The reaction mixture was refluxed for 2 h and cooled to room temperature, and the precipitated solid was collected, washed with THF (50 mL), and dried in oven to afford 10 g of compound 2 (65% yield).

### Step B

To compound 2 (14.13 g, 56.5 mmol) were added sequentially POCl₃ (86 mL, 141 g, 908 mmol) and N,N-dimethylaniline (0.69 g, 5.65 mmol). The reaction mixture was refluxed for 3 hours, during which time all of the precipitate dissolved. The resulting mixture was evaporated under reduced pressure and an ice-water mixture was added to the residue. The obtained mixture was extracted with ethyl acetate (200 mL), and the combined organic layers were washed with brine, dried over anhydrous Na₂SO₄ and evaporated under reduced pressure to obtain 12 g of compound 3 (80% yield).

### Step C

To a solution of compound 3 (5 g, 18.7 mmol) in acetonitrile (100 mL) were added K₂CO₃ (2.59 g, 18.7 mmol) and morpholine (1.632 g, 18.7 mmol). The reaction mixture was stirred for 18 h at room temperature, and was then diluted with water. The precipitated solid was collected, washed with water (100 mL), and dried in oven to afford 5 g of compound 4 (84% yield).

### Step D: 4-(2-bromo-5-(3-(m-tolyl)-1H-pyrazol-1-yl)pyrazolo[1,5-a]pyrimidin-7-yl)morpholine (Example 24)

To a solution of compound 4 (1.51 g, 4.75 mmol) in DMF (75 mL) were added K₂CO₃ (0.79 g, 5.7 mmol) and 5-(m-tolyl)-1H-pyrazole (0.752 g, 4.75 mmol). The reaction mixture was stirred for 7 h at 130° C., cooled to RT, diluted with water (75 mL), and extracted with ethyl acetate (100 mL). The combined organic layers were washed with water, dried over anhydrous Na₂SO₄ and evaporated under reduced pressure. Purification of the residue via flash column chromatography on silica gel afforded 1.5 g of Example 24 (48% yield). Mass (m/z): 441 [M+H]⁺. LCMS purity: 100%.

### Example 25

The compound of Example 25 may be prepared according to the following synthetic scheme:

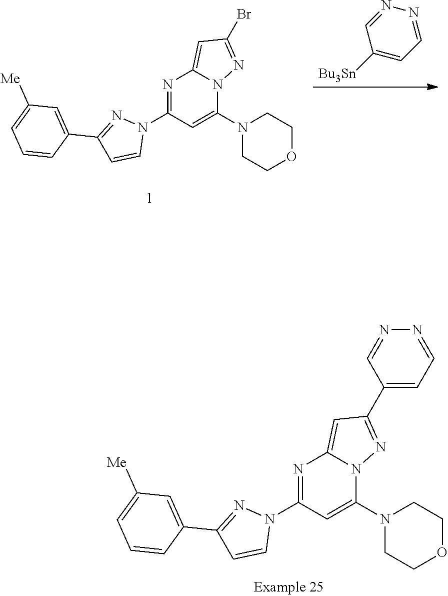

### 4-(2-(pyridazin-4-yl)-5-(3-(m-tolyl)-1H-pyrazol-1-yl)pyrazolo[1,5-a]pyrimidin-7-yl)morpholine (Example 25)

To a stirred solution of 4-(2-bromo-5-(3-(m-tolyl)-1H-pyrazol-1-yl)pyrazolo[1,5-a]pyrimidin-7-yl)morpholine (1) (25 mg, 0.057 mmol, 1 eq.) in 1,4-dioxane (3 mL) was added 4-(tributylstannyl)pyridazine (24 mg, 0.063 mmol, 1.2 eq.) at room temperature under argon. The reaction mixture was stirred for 10 min at the same temperature. After 10 min, Pd(t-Bu₃P)₂ (1.9 mg, 0.003 mmol, 6 mol %.) was added, followed by cesium fluoride (19 mg, 0.125 mmol, 2.2 eq.). The reaction was stirred at 100° C. for 12 h in a sealed tube. After completion of the reaction, it was quenched with methanol (2 mL) and evaporated under vacuum to obtain the crude product, which was purified by flash purification (Grace-C18 column of 4 g) with 80-85% acetonitrile in water as an eluent, to give the title compound Example 25 as a pale-yellow solid (9 mg, 37.5% yield). Mass (m/z): 439.2 [M+H]⁺. LCMS purity: 97.99%. ¹H-NMR (400 MHz, CDCl3): 9.78 (dd, J=1.2, 2 Hz, 1H), 9.30 (dd, J=1.6, 5.6 Hz, 2H), 8.64 (d, J=2.8 Hz, 2H), 7.94 (dd, J=2.4, 5.2 Hz, 1H), 7.76 (bs, 1H), 7.76-7.72 (m, 2H), 7.38-7.34 (m, 1H), 7.29 (br s, 1H), 7.21 (br s, 1H), 7.16 (s, 1H), 6.92 (s, 1H), 6.84 (d, J=2.4 Hz, 1H), 4.07-4.05 (m, 4H), 3.95-3.93 (m, 4H), 2.45 (s, 3H).

### Example 27

The compound of Example 27 may be prepared according to the following synthetic scheme:

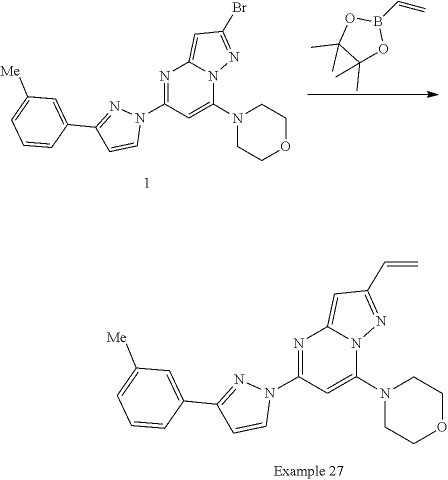 [SMILES: Cc1cccc(-c2ccn(-c3cc(N4CCOCC4)n4nc(Br)cc4n3)n2)c1 ; C=CB1OC(C)(C)C(C)(C)O1 ; C=Cc1cc2nc(-n3ccc(-c4cccc(C)c4)n3)cc(N3CCOCC3)n2n1]

### 4-(5-(3-(m-tolyl)-1H-pyrazol-1-yl)-2-vinylpyrazolo[1,5-a]pyrimidin-7-yl)morpholine (Example 27)

To a stirred solution of 4-(2-bromo-5-(3-(m-tolyl)-1H-pyrazol-1-yl)pyrazolo[1,5-a]pyrimidin-7-yl)morpholine (1) (200 mg, 0.456 mmol, 1 eq.) in DMF:water (4:1) (8 mL) was added 4,4,5,5-tetramethyl-2-vinyl-1,3,2-dioxaborolane (140 mg, 0.911 mmol, 2 eq.) and Na₂CO₃(2M) (142 mg, 0.455 mmol, 1 eq.) at room temperature under nitrogen atmosphere. The reaction mixture was degassed with nitrogen for 10-15 min, then tetrakis(triphenylphosphine)palladium(0) (53 mg, 0.045 mmol, 0.05 eq.) was added. The reaction was stirred at 100° C. for 3 h. After completion, the reaction mixture was diluted with EtOAc (20 mL) and filtered through a celite pad. The celite pad was washed with EtOAc (15 mL). The combined filtrates were evaporated under vacuum to obtain the crude product as a brown sticky compound. The crude product was purified by using silica gel (100-200) column chromatography using 80-85% EtOAc in hexane as an eluent to give the title compound Example 27 as a white solid (34 mg, 7.6% yield). Mass (m/z): 387.2 [M+H]⁺. LCMS purity: 95.63%. ¹H-NMR (400 MHz, DMSO-d₆): 8.62 (d, J=2.8 Hz, 2H), 7.75 (s, 1H), 7.72 (d, J=7.6 Hz, 1H), 7.36 (t, J=9.2 Hz, 1H), 7.20 (d, J=7.6 Hz, 1H), 7.04 (s, 1H), 6.88-6.83 (m, 1H), 6.81 (d, J=2.4 Hz, 1H), 6.55 (s, 1H), 6.03 (dd, J=1.2, 18 Hz, 1H), 5.53 (dd, J=1.2, 10.8 Hz, 1H), 4.02-4.00 (m, 4H), 3.88-3.85 (m, 4H).

### Example 26, 28 and 29

The compounds of Examples 26, 28 and 29 may be prepared according to the following synthetic scheme:

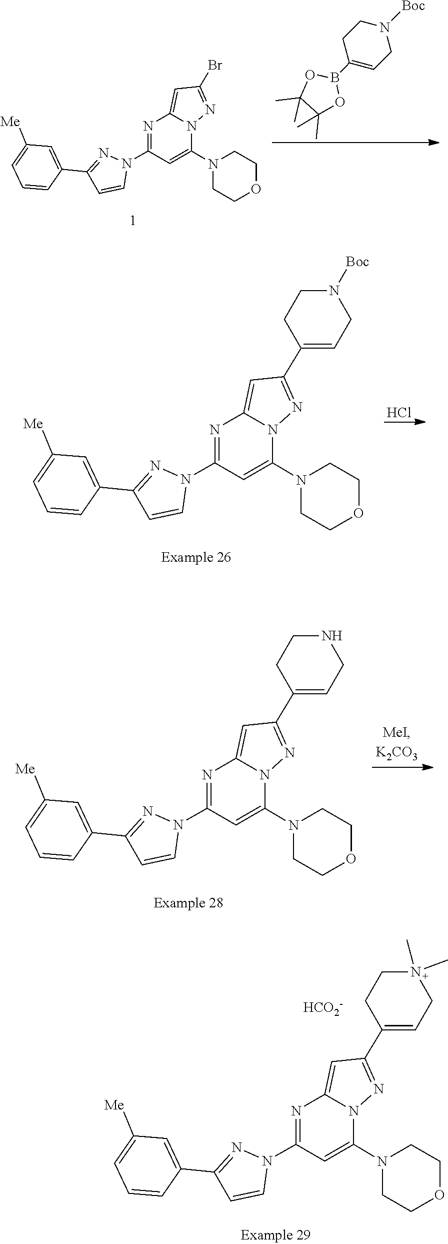

tert-butyl4-(7-morpholino-5-(3-(m-tolyl)-1H-pyrazol-1-yl)pyrazolo[1,5-a]pyrimidin-2-yl)-3,6-dihydropyridine-1(2H)-carboxylate (Example 26)

To a stirred solution of 4-(2-bromo-5-(3-(m-tolyl)-1H-pyrazol-1-yl)pyrazolo[1,5-a]pyrimidin-7-yl)morpholine (1) (100 mg, 0.228 mmol, 1 eq.) in 1,4-dioxane:water (4:1) (5 mL) was added tert-butyl 4-(4,4,5,5-tetramethyl-1,3,2-dioxaborolan-2-yl)-3,6-dihydropyridine-1(2H)-carboxylate (110 mg, 0.342 mmol, 1.5 eq.), NaHCO₃ (1.2M aqueous) (57 mg, 0.683 mmol, 3 eq.) at room temperature under nitrogen atmosphere. The reaction mixture was degassed with N₂ for 15 min, then Pd(dppf)Cl₂·DCM (9.5 mg, 0.014 mmol, 0.05 eq.) was added and the reaction was degassed for another 5-10 min. Then the reaction was then heated to 120° C. for 1 h in a microwave. After completion, the reaction mixture was diluted with EtOAc, filtered through a celite pad, and washed with EtOAC (5 mL). The filtrate was evaporated under vacuum to obtain the crude product as brown sticky compound. The crude product was purified by flash purification (Grace-C18 column of 4 g) with 80-85% acetonitrile in water as an eluent to give the title compound Example 26 as a white solid (30 mg, 24.3% yield). Mass (m/z): 542.3 [M+H]⁺. LCMS purity: 98.35%. ¹H-NMR (400 MHz, DMSO-d₆): 8.61 (d, J=2.8 Hz, 1H), 7.75 (s, 1H), 7.72 (d, J=7.6 Hz, 1H), 7.34 (t, J=7.6 Hz, 1H), 7.20 (d, J=7.6 Hz, 1H), 7.01 (s, 1H), 6.81 (d, J=2.8 Hz 1H), 6.49 (s, 2H), 4.14 (br s, 2H), 4.02-4.00 (m, 4H), 3.89-3.87 (m, 4H), 3.66 (d, J=5.6 Hz 2H), 2.68 (br s, 2H), 2.44 (s, 3H), 1.50 (s, 9H).

### 4-(2-(1,2,3,6-tetrahydropyridin-4-yl)-5-(3-(m-tolyl)-1H-pyrazol-1-yl)pyrazolo[1,5-a]pyrimidin-7-yl)morpholine (Example 28)

To a stirred solution of tert-butyl 4-(7-morpholino-5-(3-(m-tolyl)-1H-pyrazol-1-yl) pyrazolo[1,5-a]pyrimidin-2-yl)-3,6-dihydropyridine-1(2H)-carboxylate (25 mg, 0.046 mmol, 1 eq.) in 1,4-dioxane (2 mL) was added 4N HCl in 1,4-dioxane (0.3 mL, 0.230 mmol, 5 eq.) at 0° C., and the reaction was allowed to warm to room temperature and stir for 6 h. After completion, the reaction mixture was evaporated under vacuum to obtain the crude product, which was triturated with diethyl ether (5 mL) and n-pentane (10 mL) to give the title compound Example 28 as an off-white solid (17 mg, 85% yield). Mass (m/z): 442.2 [M+H]⁺. LCMS purity: 98.37%. ¹H-NMR (400 MHz, DMSO-d₆): 9.04 (b s, 2H), 8.68 (d, J=2.8 Hz, 1H), 7.81-7.79 (d, J=9.2 Hz, 2H), 7.39 (t, J=14.8 Hz, 1H), 7.24 (d, J=7.6 Hz, 1H), 7.13 (d, J=2.8 Hz, 1H), 6.96 (s, 1H), 6.78 (s, 1H), 6.59 (b s, 1H), 3.88 (s, 8H), 3.82 (b s, 2H), 3.38-3.35 (b s, 2H), 2.80 (bs, 2H), 2.40 (s, 3H).

### 1,1-dimethyl-4-(7-morpholino-5-(3-(m-tolyl)-1H-pyrazol-1-yl)pyrazolo[1,5-a]pyrimidin-2-yl)-1,2,3,6-tetrahydropyridin-1-ium (Example 29)

To a stirred solution of 4-(2-(1,2,3,6-tetrahydropyridin-4-yl)-5-(3-(m-tolyl)-1H-pyrazol-1-yl)pyrazolo[1,5-a]pyrimidin-7-yl)morpholine (10 mg, 0.022 mmol, 1 eq.) in DMF (2 mL) was added K₂CO₃ (7 mg, 0.045 mmol, 1.5 eq.) at 0° C., and the reaction mixture was stirred for 10 min. Iodomethane (5 mg, 0.034 mmol, 2 eq.) was added dropwise at the same temperature. The reaction mixture was allowed to warm to room temperature and stir for 12 h in a sealed tube. After completion, the reaction was evaporated under vacuum and triturated with Et₂O (5 mL) to obtain the crude material, which was purified by preparative HPLC to give the title compound Example 29 as a light brown fluffy solid (4.5 mg, 45% yield). Mass (m/z): 470.3 [M+H]⁺. LCMS purity: 96.29%. ¹H-NMR (400 MHz, DMSO-d₆): 8.70 (d, J=2.8 Hz, 1H), 7.82-7.80 (m, 2H), 7.40 (t, J=15.2 Hz, 1H), 7.25-7.21 (m, 1H), 7.14 (d, J=2.8 Hz, 1H), 6.99 (s, 1H), 6.83 (s, 1H), 6.59 (b s, 1H), 4.17 (s, 2H), 3.89 (b s, 8H), 3.64 (t, J=12.8 Hz, 2H), 3.16 (s, 6H), 2.98 (bs, 2H), 2.40 (s, 3H).

### Example 30

The compound of Example 30 may be prepared according to the following synthetic scheme:

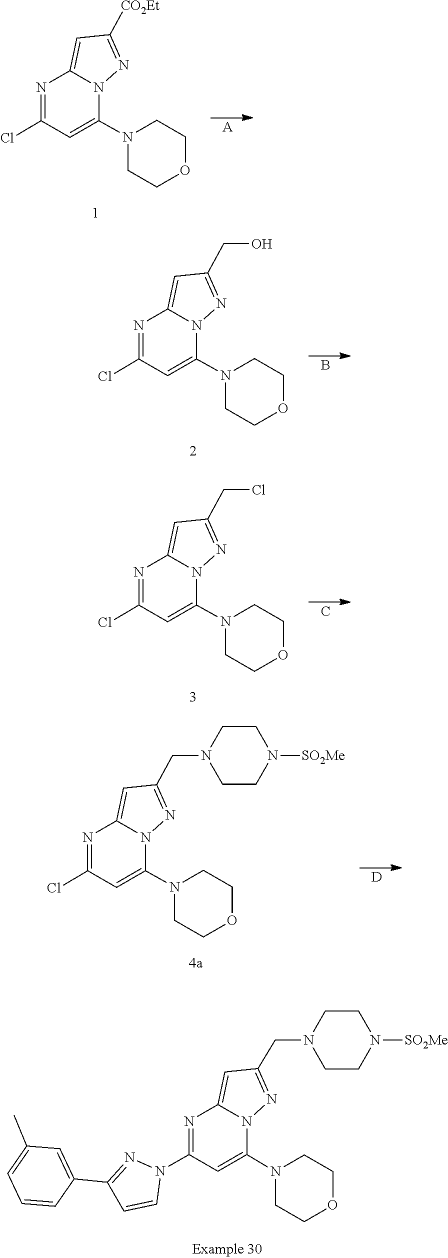

### Step A

To a stirred solution of ethyl 5-chloro-7-morpholinopyrazolo[1,5-a]pyrimidine-2-carboxylate (1) (100 mg, 0.32 mmol, 1 eq.) in THF (5 mL) at −78° C. was added diisobutylaluminum hydride (1.6 mL, 1.6 mmol, 5 eq). The reaction mixture was stirred at same temperature for 2 h and the progress of the reaction was monitored by TLC. After completion of the reaction, it was quenched with saturated NH₄Cl solution (20 mL) and extracted with EtOAc (2×20 mL). Finally, the combined organic layer was washed with water (10 mL) dried over anhydrous Na₂SO₄, filtered and evaporated under reduced pressure to afford (5-chloro-7-morpholinopyrazolo[1,5-a]pyrimidin-2-yl)methanol (2) as a white solid (80 mg, 93%).

### Step B

To a stirred solution of (2) (80 mg, 0.29 mmol, 1 eq.), in DCM (5 mL) at 0° C. was added SOCl₂ (0.1 mL). The reaction mixture was allowed to warm to room temperature and was stirred for 2 h. The progress of the reaction was monitored by TLC and after completion, it was evaporated under reduced pressure to provide 4-(5-chloro-2-(chloromethyl)pyrazolo[1,5-a]pyrimidin-7-yl)morpholine (3) as a white solid (70 mg, 76%).

### Step C

To a stirred solution of 4 (3) (60 mg, 0.2 mmol, 1 eq.) in THF (5 mL) was added 1-(methylsulfonyl)piperazine (51 mg, 0.3 mmol, 1.5 eq) and K₂CO₃ (86 mg, 0.6 mmol, 3 eq) at room temperature. The reaction mixture was heated to 100° C. and maintained at this temperature for 16 h. Progress of the reaction was monitored by TLC. After completion of the reaction, it was diluted with EtOAc (20 mL) and washed with water (10 mL). Finally, the organic layer was dried over anhydrous Na₂SO₄, filtered and evaporated under reduced pressure to provide 4-(5-chloro-2-((4-(methylsulfonyl)piperazin-1-yl)methyl)pyrazolo[1,5-a]pyrimidin-7-yl)morpholine (4a) as a brown gummy gel (80 mg, 69%).

### Step D: 4-(2-((4-(methylsulfonyl)piperazin-1-yl)methyl)-5-(3-(m-tolyl)-1H-pyrazol-1-yl)pyrazolo[1,5-a]pyrimidin-7-yl)morpholine (Example 30)

A solution of t-ButylXphos (4.6 mg, 0.01 mmol, 0.15 eq), and Pd2(dba)₃ (20 mg, 0.02 mmol, 0.3 eq) in toluene (2 mL) was degassed for 10 min and heated to 100° C. for 5 min, until the solution turns clear. This solution is then added slowly to a degassed mixture of (4) (30 mg, 0.07 mmol, 1 eq.), 3-(m-tolyl)-1H-pyrazole (18 mg, 0.1 mmol, 1.5 eq) and K₃PO₄ (46 mg, 0.2 mmol, 3 eq) in 1,4-dioxane (2 mL). This reaction mixture was heated to 120° C. and stirred for 5 h. The progress of the reaction was monitored by TLC and after completion, it was diluted with EtOAc (20 mL) and washed with water (10 mL). Finally, the organic layer was dried over anhydrous Na₂SO₄, filtered and evaporated under reduced pressure to obtain crude (50 mg) as brownish gummy material. The crude compound was purified by preparative HPLC (Method-B: Kinetex, EVO, C18, 250×21.2 mm, 5 μm; Mobile phase: [ACN: 0.1% of Formic acid in Water]; time/B %: 0/15, 12/40, 15/50, 17/95), and after lyophilization afforded product (Example 30) as an off-white solid (12 mg, 31%). Mass (m/z): 537.2 [M+H]⁺. LCMS purity >99%. ¹H-NMR (400 MHz, DMSO-ds): 8.68 (d, J=2.4 Hz, 1H), 7.81-7.87 (m, 1H), 7.39 (t, J=7.6 Hz, 1H), 7.23-7.21 (m, 2H), 7.12 (d, J=2.4 Hz, 1H), 6.95 (s, 1H), 6.42 (s, 1H), 3.86 (br s, 6H), 3.72 (br s, 2H), 3.13 (br s, 4H), 2.87 (s, 3H), 2.5 (br s, 4H), 2.40 (s, 3H).

### Example 31

The compound of Example 31 may be prepared according to the following synthetic scheme:

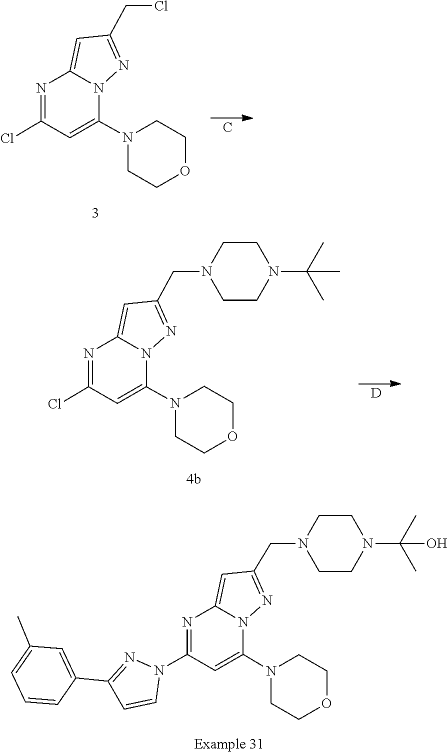

### Step C

To a stirred solution of (3) (50 mg, 0.17 mmol, 1 eq.) in THF (5 mL) was added 2-(piperidin-4-yl)propan-2-ol (25 mg, 0.17 mmol, 1 eq) and K₂CO₃ (70 mg, 0.51 mmol, 3 eq). The reaction mixture was stirred 16 h at 100° C. The progress of the reaction was monitored by TLC and after completion, it was diluted with EtOAc (20 mL) and washed with water (10 mL). Finally, the organic layer was dried over anhydrous Na₂SO₄, filtered and evaporated under reduced pressure to afford product 2-(1-((5-chloro-7-morpholinopyrazolo[1,5-a]pyrimidin-2-yl)methyl)piperidin-4-yl)propan-2-ol (4b) as a brown sticky solid (35 mg, 51%).

### Step D: 2-(1-((7-morpholino-5-(3-(m-tolyl)-1H-pyrazol-1-yl)pyrazolo[1,5-a]pyrimidin-2-yl)methyl)piperidin-4-yl)propan-2-ol (Example 31)

A solution of t-ButylXphos (2.8 mg, 0.003 mmol, 0.15 eq), and Pd2(dba)₃ (6 mg, 0.007 mmol, 0.3 eq) in toluene (2 mL) was degassed for 10 min and heated to 100° C. for 5 min, until the solution turns clear. This solution was added slowly to a degassed mixture of 2 (4b) (10 mg, 0.02 mmol, 1 eq.), 3-(m-tolyl)-1H-pyrazole (1.6 mg, 0.027 mmol, 1.5 eq) and K₃PO₄ (12 mg, 0.7 mmol, 3 eq) in 1,4-dioxane (2 mL). This reaction mixture was heated to 120° C. and stirred for 5 h. The progress of the reaction was monitored by TLC and after completion, it was diluted with EtOAc (20 mL) and washed with water (10 mL). Finally, the organic layer was dried over anhydrous Na₂SO₄, filtered and evaporated under reduced pressure to obtain crude (50 mg) as brown gel. The crude compound was purified by Prep HPLC (Method-B: Kinetex, EVO, C18, 250×21.2 mm, 5 μm; Mobile phase: [ACN: 0.1% of Formic acid in Water]; time/B %: 0/15, 12/40, 15/50, 17/95), and after lyophilization afforded product (Example 31) as an off-white solid (6 mg, 46%). Mass (m/z): 516.3 [M+H]⁺. LCMS purity 99%. ¹H-NMR (400 MHz, DMSO-d₆): 8.69 (d, J=2.8 Hz, 1H), 7.82-7.79 (m, 2H), 7.39 (t, J=7.6 Hz, 1H), 7.23-7.22 (d, J=6.8 Hz 1H), 7.13 (d, J=2.4 Hz, 1H), 6.93 (s, 1H), 6.39 (s, 1H), 3.86 (br s, 8H), 3.60 (s, 2H), 2.96-2.93 (m, 2H), 2.40 (s, 3H), 1.94-1.89 (m, 2H), 1.65-1.62 (m, 2H), 1.26-1.17 (m, 2H), 1.02 (s, 6H).

### Example 32

The compound of Example 32 may be prepared according to the following synthetic scheme:

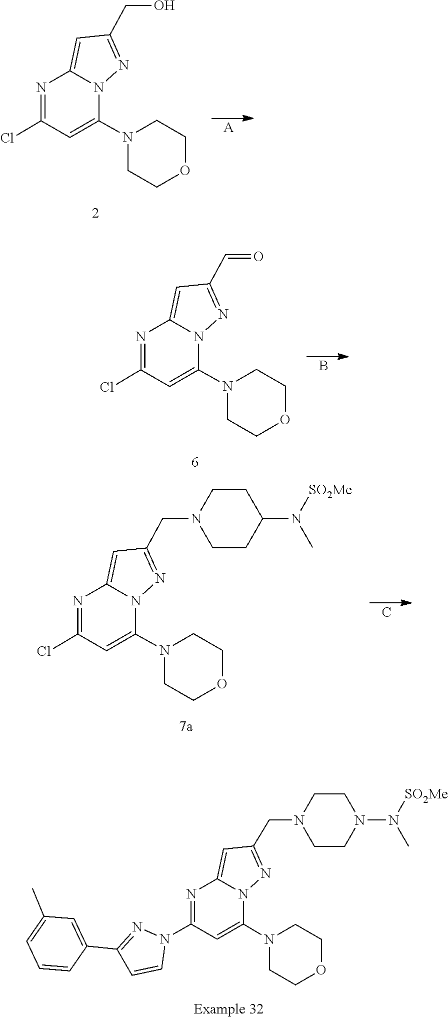

### Step A

To a stirring solution of (5-chloro-7-morpholinopyrazolo[1,5-a]pyrimidin-2-yl)methanol (2) (200 mg, 0.74 mmol, 1 eq.) in DCM (5 mL) at 0° C. was added Dess-martin periodinane (474 mg, 1.11 mmol, 1.5 eq). The reaction temperature was allowed to warm to 25° C. and was stirred for 4 h. The progress of the reaction was monitored by TLC and after completion, the reaction mixture was diluted with DCM (20 mL). It was then filtered through a celite bed to remove inorganics and the filtrate was washed with saturated NaHCO₃ solution (2×50 mL). The organic layer was dried over anhydrous Na₂SO₄, filtered and evaporated to provide the crude product as an off white solid (100 mg). The crude product was purified through a silica gel column (60-120) chromatography using 40-50% EtOAc in hexane as an eluent to 5-chloro-7-morpholinopyrazolo[1,5-a]pyrimidine-2-carbaldehyde (6) as a white solid (100 mg, 50%)

### Step B

To a stirring solution of (6) (50 mg, 0.18 mmol, 1 eq.), and N-methyl-N-(piperidin-4-yl)methanesulfonamide (55 mg, 0.28 mmol, 1.5 eq) in dichloroethane (DCE) (5 mL) was added with titanium isopropoxide (52 mg, 0.28 mmol, 1.5 eq) at room temperature. The reaction mixture was heated to 80° C. and stirred for 16 h. It was then cooled to 0° C. and NaBH₃CN (18 mg, 0.28 mmol, 1.5 eq) was added. The reaction mixture temperature was allowed to warm to 25-30° C. and was stirred for a further 16 h. The progress of the reaction was monitored by TLC and after completion, it was diluted with DCM (20 mL) and washed with water (2×10 mL). Finally, the organic layer was dried over anhydrous Na₂SO₄, filtered and evaporated to produce crude N-(1-((5-chloro-7-morpholinopyrazolo[1,5-a]pyrimidin-2-yl)methyl)piperidin-4-yl)-N-methylmethanesulfonamide (7a) as a white solid (40 mg, 96%).

### Step C: N-methyl-N-(1-((7-morpholino-5-(3-(m-tolyl)-1H-pyrazol-1-yl)pyrazolo[1,5-a]pyrimidin-2-yl)methyl)piperidin-4-yl)methanesulfonamide (Example 32)

To a stirred solution of (7a) (40 mg, 0.09 mmol, 1 eq.), and 3-(m-tolyl)-1H-pyrazole (14 mg, 0.09 mmol, 1 eq) in THF (5 mL) was added sodium hydride (7 mg, 0.27 mmol, 3 eq) and the reaction mixture was stirred for 16 h at 90° C. in a sealed tube. The progress of the reaction was monitored by TLC and after completion, it was diluted with EtOAc (20 mL) followed by extraction with water (2×10 mL). Finally, the organic layer was dried over anhydrous Na₂SO₄, filtered and evaporated under reduced pressure to obtain the crude (50 mg) as an off-white solid. The crude product was purified by preparative HPLC (Method-B: Kinetex, EVO, C18, 250×21.2 mm, 5 μm; Mobile phase: [ACN: 0.1% of Formic acid in Water]; time/B %: 0/15, 12/40, 17/50, 20/95), and after lyophilization afforded the product (Example 32) as off-white solid (4 mg, 7%). Mass (m/z): 565.3 [M+H]⁺. LCMS purity 95%. ¹H-NMR (400 MHz, DMSO-d₆): 8.69 (d, J=2.8 Hz, 1H), 7.82-7.79 (m, 1H), 7.39 (t, J=7.6 Hz, 1H), 7.23-7.21 (m, 1H), 7.13 (d, J=2.8 Hz, 1H), 6.94 (s, 1H), 6.42 (s, 1H), 3.86 (s, 8H), 3.65 (s, 2H), 3.57-3.54 (m, 1H), 2.97-2.94 (m, 2H), 2.89 (s, 3H), 2.69 (s, 3H), 2.40 (s, 3H), 2.13-2.07 (m, 2H), 1.75-1.70 (m, 2H), 1.60-1.57 (m, 2H).

### Example 33

The compound of Example 33 may be prepared according to the following synthetic scheme:

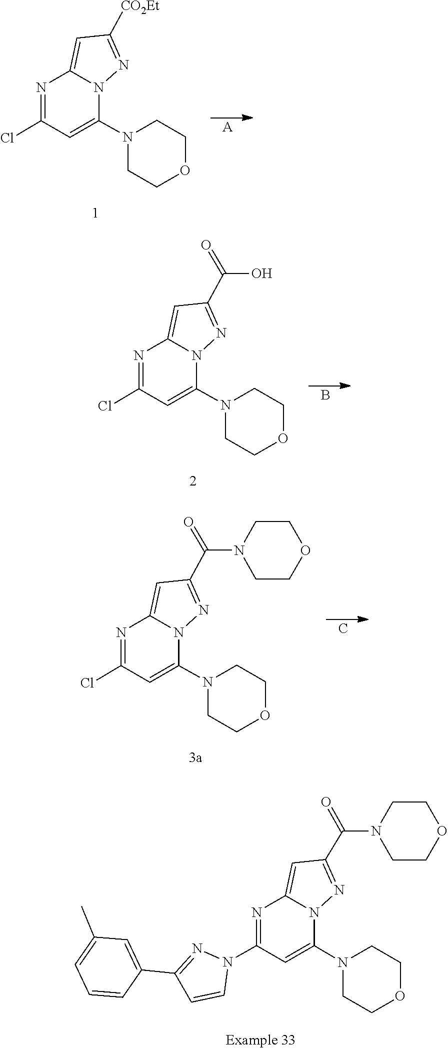

### Step A

A stirred solution of ethyl 5-chloro-7-morpholinopyrazolo[1,5-a]pyrimidine-2-carboxylate (1) (200 mg, 0.645 mmol, 1 eq.) in THF:water (1:1) (5 mL) was cooled to 0° C. and LiOH (81 mg, 1.93 mmol, 3.0 eq.) was added. The reaction was stirred at room temperature for 4 h and progress of the reaction was monitored by TLC. After completion, it was evaporated under vacuum to obtain a residue. This was diluted with water (5 mL) and acidified with 2N HCl up to a pH of about 2-3, at which point a solid precipitated out. The solid was filtered and dried to obtain 5-chloro-7-morpholinopyrazolo[1,5-a]pyrimidine-2-carboxylic acid (2) as a light brown solid (144 mg, 77%).

### Step B

To a stirred solution of (2) (50 mg, 0.17 mmol, 1 eq.) and morpholine (23 μL, 0.26 mmol, 1.5 eq.) in DMF (5 mL) was added DIPEA (98 μL, 0.53 mmol, 3.0 eq.) and HATU (101 mg, 0.26 mmol, 1.5 eq.). The reaction was stirred at room temperature for 18 h and the progress of the reaction was monitored by TLC. After completion, the reaction was diluted with EtOAc (30 mL) and washed with ice cold water (2×10 mL), then brine solution (1×20 mL). The organic layer was dried over Na₂SO₄, filtered and evaporated under vacuum to obtain the crude product as a brown sticky solid. This crude was purified by using silica gel (60-120) column chromatography using 50-55% EtOAc in hexane as an eluent to afford (5-chloro-7-morpholinopyrazolo[1,5-a]pyrimidin-2-yl)(morpholino)methanone (3a) as an off white solid (55 mg, 88%)

### Step C: Morpholino(7-morpholino-5-(3-(m-tolyl)-1H-pyrazol-1-yl)pyrazolo[1,5-a]pyrimidin-2-yl)methanone (4a) (Example 33)

To a stirred suspension of sodium hydride (57-63%) (11 mg, 0.284 mmol, 2.0 eq) in THF (2 mL) was added 3-(m-tolyl)-1H-pyrazole (22 mg, 0.142 mmol, 1.0 eq) at 0° C. under N₂ atmosphere and the reaction was stirred for 30 min. (3a) (50 mg, 0.142 mmol, 1 eq) was added and the reaction mixture was irradiated at 80° C. in a microwave for 2 h. The progress of the reaction was monitored by TLC and after completion, it was quenched with ice-cold water (5 mL) and extracted with ethyl acetate (3×10 mL). The combined organic layer was washed with brine solution (5 mL), dried over anhydrous Na₂SO₄, and filtered. It was then evaporated under reduced pressure to provide crude material which was purified through flash chromatography (C18; reverse phase column (12 g)) using 20-40% (CH₃CN/H₂O) acetonitrile and water as eluent. The collected fractions were distilled to provide the product (Example 33) (2.0 mg) as an off-white fluffy solid (2.0 mg, 3%). Mass (m/z): 474.2 [M+H]⁺. LCMS purity 98%. ¹H NMR (400 MHz, DMSO-ds): 8.72 (d, J=2.8 Hz, 1H), 7.83-7.80 (m, 2H), 7.40 (t, J=7.6 Hz, 1H), 7.24 (d, J=7.2 Hz, 1H), 7.15 (d, J=2.8 Hz, 1H), 7.06 (s, 1H), 6.74 (s, 1H), 3.86-3.80 (m, 10H), 3.68-3.50 (m, 6H), 2.40 (s, 3H).

### Example 34

The compound of Example 34 may be prepared according to the following synthetic scheme:

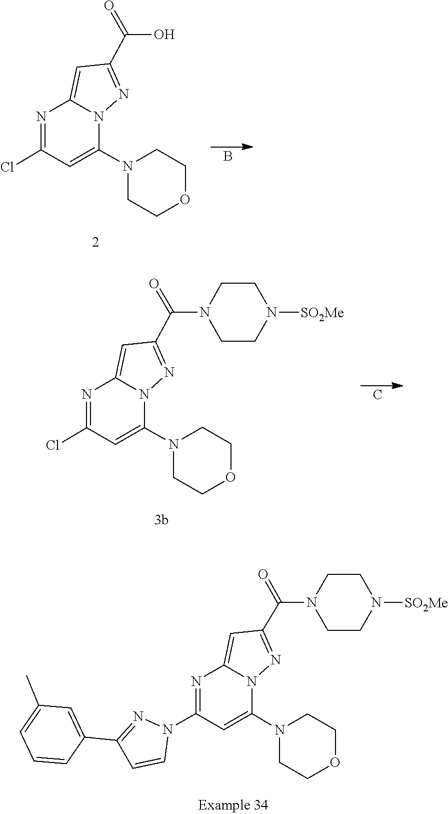

### Step B

To a stirred solution of (2) (50 mg, 0.17 mmol, 1 eq.) and 1-(methyl sulfonyl)piperazine (43 mg, 0.26 mmol, 1.5 eq.) in DMF (5 mL) was added DIPEA (0.1 mL, 0.53 mmol, 3.0 eq.) along with HATU (101 mg, 0.26 mmol, 1.5 eq.). The reaction was stirred at room temperature for 18 h and the progress of the reaction was monitored by TLC. After completion, the reaction mixture was diluted with EtOAc (30 mL) and washed with ice cold water (2×10 mL) followed by brine solution (10 mL). The organic layer was dried over Na₂SO₄, filtered and evaporated under vacuum to obtain crude (5-chloro-7-morpholinopyrazolo[1,5-a]pyrimidin-2-yl)(4-(methylsulfonyl)piperazin-1-yl)methanone (3b) as a brown sticky solid (65 mg, 86%).

### Step C: (4-(methylsulfonyl)piperazin-1-yl)(7-morpholino-5-(3-(m-tolyl)-1H-pyrazol-1-yl)pyrazolo[1,5-a]pyrimidin-2-yl)methanone (Example 34)

In a sealed tube, a stirred suspension of sodium hydride (57-63%) (9 mg, 0.23 mmol, 2.0 eq) in THF (2 mL) was added 3-(m-tolyl)-1H-pyrazole (19 mg, 0.116 mmol, 1.0 eq) at 0° C. under N₂ atmosphere and the reaction was stirred for 30 min. (3b) (50 mg, 0.116 mmol, 1 eq) was then added at the same temperature and the reaction was irradiated at 80° C. in a microwave for 2 h. The progress of the reaction was monitored by TLC and after completion, it was quenched with ice-cold water (5 mL) and extracted with ethyl acetate (3×10 mL). The combined organic layers were washed with brine solution (5 mL) and the collected organic layer was dried over anhydrous Na₂SO₄. It was then evaporated under reduced pressure to provide a crude material which was purified by flash chromatography (C18-reverse phase 12 g column) and eluted using 40-55% (ACN/H₂O) acetonitrile and water as an eluent to provide the product (Example 34) as an off-white solid. (2.5 mg, 4%). Mass (m/z): 551.3 [M+H]⁺. LCMS purity 95%. ¹H NMR (400 MHz, DMSO-d₆): 8.73 (d, J=2.8 Hz, 1H), 7.83-7.80 (m, 2H), 7.40 (t, J=7.6 Hz, 1H), 7.24 (d, J=7.2 Hz, 1H), 7.16 (d, J=2.8 Hz, 1H), 7.08 (s, 1H), 6.76 (s, 1H), 3.93-3.80 (m, 12H), 3.29-3.20 (m, 4H), 2.93 (s, 3H), 2.40 (s, 3H).

### Example 35

The compound of Example 35 may be prepared according to the following synthetic scheme:

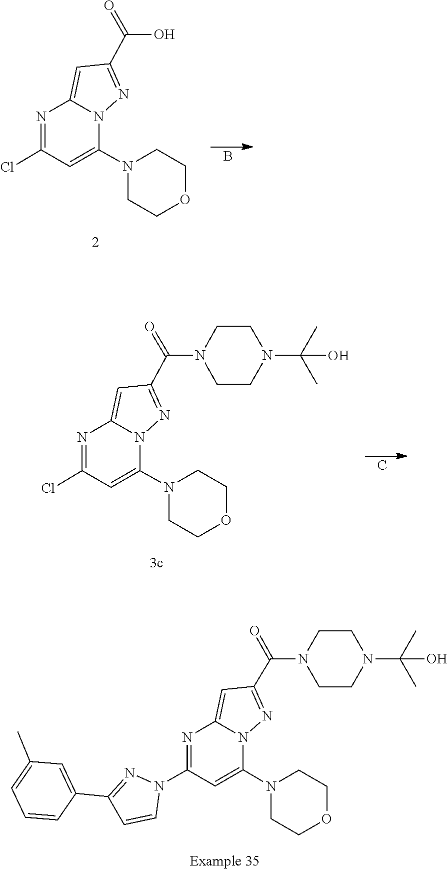

### Step B

To a stirred solution of (2) (50 mg, 0.17 mmol, 1 eq.) and 2-(piperidin-4-yl)propan-2-ol (38 mg, 0.26 mmol, 1.5 eq.) in DMF (5 mL) was added DIPEA (0.1 mL, 0.53 mmol, 3.0 eq.) along with HATU (101 mg, 0.26 mmol, 1.5 eq.). The reaction mass was stirred at room temperature for 18 h and progress of the reaction was monitored by TLC. After completion of the reaction, it was diluted with EtOAc (30 mL) and washed with ice cold water (2×20 mL) followed by a brine wash (10 mL). The collected organic layer was dried over Na₂SO₄, filtered and evaporated under vacuum to obtain crude (5-chloro-7-morpholinopyrazolo[1,5-a]pyrimidin-2-yl)(4-(2-hydroxypropan-2-yl)piperidin-1-yl)methanone (3c) as a brown sticky liquid (55 mg, 76%).

### Step C: (4-(methylsulfonyl)piperazin-1-yl)(7-morpholino-5-(3-(m-tolyl)-1H-pyrazol-1-yl)pyrazolo[1,5-a]pyrimidin-2-yl)methanone (Example 35)

To a stirred suspension of sodium hydride (57-63%) (9 mg, 0.23 mmol, 2.0 eq) in THF (2 mL) was added 3-(m-tolyl)-1H-pyrazole (19 mg, 0.116 mmol, 1.0 eq) at 0° C. under N₂ atmosphere and the reaction was stirred for 30 min. (3c) (50 mg, 0.116 mmol, 1 eq) was added at the same temperature and the reaction mixture was irradiated at 80° C. in a microwave for 2 h. After completion of the reaction, it was quenched with ice-cold water (5 mL) and extracted with ethyl acetate (3×10 mL). The combined organic layers were washed with brine solution (5 mL) and the organic layer was dried over anhydrous Na₂SO₄. It was then evaporated under reduced pressure to provide a crude material which was purified by flash chromatography (C18-reverse phase 12 g column) using 30-40% (CH₃CN/H₂O) acetonitrile in water as an eluent to provide the product (Example 35) as an off-white solid (12 mg, 18%). Mass (m/z): 530.3 [M+H]⁺. LCMS purity 97%. ¹H NMR (400 MHz, DMSO-d₆): 8.72 (d, J=2.4 Hz, 1H), 7.83 (bs, 1H), 7.80 (bs, 1H), 7.40 (t, J=14.4 Hz, 1H), 7.24 (d, J=8.0 Hz, 1H), 7.15 (d, J=2.8 Hz, 1H), 7.05 (s, 1H), 6.69 (s, 1H), 4.64 (d, J=12.8 Hz, 1H), 4.38 (d, J=12.4 Hz, 1H), 4.21 (s, 1H), 3.86 (s, 8H), 3.04 (t, J=11.6 Hz, 1H), 2.72-2.66 (m, 2H), 3.29-3.20 (m, 4H), 2.45-2.43 (m, 3H), 2.40 (s, 3H), 1.84-1.72 (dd, J=12.4, J=14 Hz, 2H), 1.54-1.48 (t, J=11.6, 1H). 1.29-1.17 (m, 2H), 1.05 (s, 6H).

### Example 36

The compound of Example 36 may be prepared according to the following synthetic scheme:

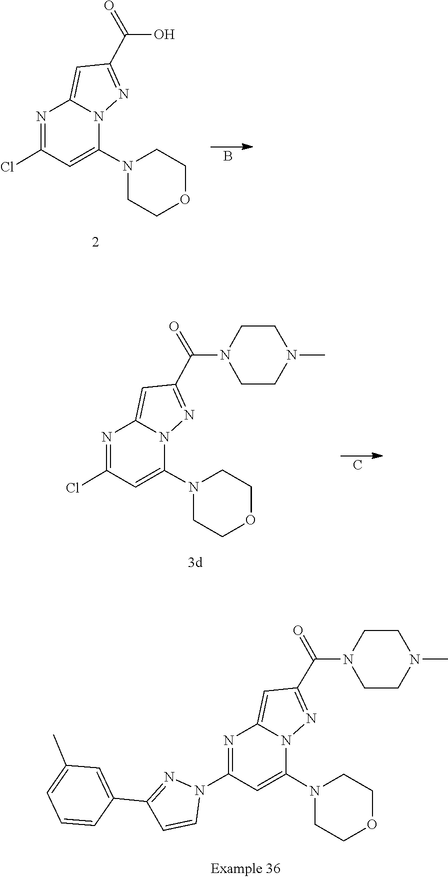

### Step B

To a stirred solution of (2) (50 mg, 0.17 mmol, 1 eq.), and 1-methylpiperazine (26 mg, 0.26 mmol, 1.5 eq.) in DMF (5 mL) was added DIPEA (0.1 mL, 0.53 mmol, 3.0 eq.) and HATU (101 mg, 0.26 mmol, 1.5 eq.). The reaction was stirred at room temperature for 18 h and the progress of the reaction was monitored by TLC. After completion, it was diluted with EtOAc (30 mL), washed with ice cold water (2×20 mL) and then with brine solution (10 mL). Finally, the organic layer was dried over Na₂SO₄, filtered and evaporated under vacuum to obtain (5-chloro-7-morpholinopyrazolo[1,5-a]pyrimidin-2-yl)(4-methylpiperazin-1-yl)methanone (3d) as a brown sticky liquid (45 mg, 70%).

### Step C: (4-(methylsulfonyl)piperazin-1-yl)(7-morpholino-5-(3-(m-tolyl)-1H-pyrazol-1-yl)pyrazolo[1,5-a]pyrimidin-2-yl)methanone (Example 36)

A solution of t-ButylXphos (9.3 mg, 0.02 mmol, 0.2 eq), and Pd2(dba)₃ (10 mg, 0.01 mmol, 0.1 eq) in toluene (2 mL) was degassed for 10 min and heated to 100° C. for 5 min, until solution turns clear. This solution is slowly added to a degassed mixture of (3d) (40 mg, 0.1 mmol, 1 eq.), 3-(m-tolyl)-1H-pyrazole (17 mg, 0.1 mmol, 1.0 eq) and K₃PO₄ (46 mg, 0.2 mmol, 2 eq) in 1,4-dioxane (2 mL). This reaction mixture was heated to 120° C. and stirred for 5 h. The progress of the reaction was monitored by TLC and after completion, it was diluted with EtOAc (30 mL) and washed with water (10 mL). Finally, the organic layer was dried over anhydrous Na₂SO₄, filtered and evaporated under reduced pressure to obtain a crude product (50 mg) as brown liquid. The crude compound was purified by flash chromatography (C18 reverse phase 12 g column) using 40-60% acetonitrile and water as eluent to afford the product (Example 36) as an off-white solid (9 mg, 9%). Mass (m/z): 487.3 [M+H]⁺. LCMS purity 99%. ¹H NMR (400 MHz, DMSO-d₆): 8.72 (d, J=2.8 Hz, 1H), 7.83 (bs, 1H), 7.80 (bs, 1H), 7.40 (t, J=7.6 Hz, 1H), 7.24 (d, J=7.2 Hz, 1H), 7.15 (d, J=2.8 Hz, 1H), 7.06 (s, 1H), 6.71 (s, 1H), 3.86 (brs, 8H), 3.76 (brs, 2H), 3.67 (brs, 2H), 2.40 (s, 3H) 2.35-2.33 (m, 4H), 2.21 (s, 3H).

### Example 37

The compound of Example 37 may be prepared according to the following synthetic scheme:

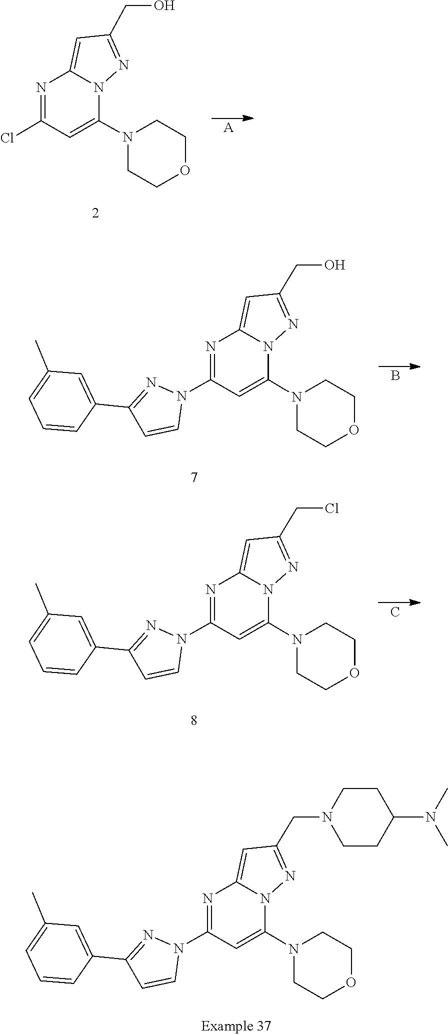

### Step A

A solution of t-butylXphos (38 mg, 0.08 mmol, 0.4 eq), and Pd2(dba)₃ (41 mg, 0.02 mmol, 0.2 eq) in toluene (2 mL) was degassed for 10 min and heated to 100° C. for 5 min, until the solution turns clear. This solution is added slowly to a degassed mixture of (5-chloro-7-morpholinopyrazolo[1,5-a]pyrimidin-2-yl)methanol (2) (60 mg, 0.22 mmol, 1 eq.), 3-(m-tolyl)-1H-pyrazole (35 mg, 0.22 mmol, 1.1 eq) and K₃PO₄ (95 mg, 0.44 mmol, 2.0 eq) in 1,4-dioxane (2 mL). This reaction mixture was heated to 120° C. and stirred for 5 h. The progress of the reaction was monitored by TLC and after completion, it was diluted with EtOAc (20 mL) and washed with water (10 mL). Finally, the organic layer was dried over anhydrous Na₂SO₄, filtered and evaporated under reduced pressure to obtain the crude product (50 mg) as a brown gum. The crude product was purified by silica gel column (60-120) chromatography eluting with 0-5% MeOH in DCM. The product fractions were concentrated and evaporated to afford (7-morpholino-5-(3-(m-tolyl)-1H-pyrazol-1-yl)pyrazolo[1,5-a]pyrimidin-2-yl)methanol (7) as a white solid (80 mg, 91%).

### Step B

To a stirred solution of (7) (80 mg, 12.8 mmol, 1 eq.), in DCM (5 mL) at 0° C. was added SOCl₂ (0.1 mL). The reaction mixture was allowed to warm to room temperature and was then stirred for 2 h. Progress of the reaction was monitored by TLC and after completion, it was evaporated under reduced pressure to provide 4-(2-(chloromethyl)-5-(3-(m-tolyl)-1H-pyrazol-1-yl)pyrazolo[1,5-a]pyrimidin-7-yl)morpholine (8) as a white solid (80 mg, 96%).

### Step C: N,N-dimethyl-1-((7-morpholino-5-(3-(m-tolyl)-1H-pyrazol-1-yl)pyrazolo[1,5-a]pyrimidin-2-yl)methyl)piperidin-4-amine (Example 37)

To a stirred solution of (8) (80 mg, 0.19 mmol, 1 eq.) in THF (5 mL) was added N,N-dimethylpiperidin-4-amine (50 mg, 0.39 mmol, 1 eq), and K₂CO₃ (54 mg, 0.39 mmol, 2 eq). The reaction mixture was heated to 100° C. for 16 h. Progress of the reaction was monitored by TLC. After completion of the reaction, it was diluted with EtOAc (20 mL) and washed with water (10 mL). Finally, the organic layer was dried over anhydrous Na₂SO₄, filtered and evaporated under reduced pressure to obtain a crude product (100 mg) as a brown gum. The obtained crude was purified by preparative HPLC (Method-B: Kinetex, EVO, C18, 250×21.2 mm, 5 μm; Mobile phase: [ACN: 0.1% of Formic acid in Water]; time/B %: 0/30, 10/50, 25/85), and after lyophilization afforded the product (Example 37) as a white solid (10 mg, 10%). Mass (m/z): 501.3 [M+H]⁺. LCMS purity 99%. ¹H-NMR (400 MHz, DMSO-d₆): 8.68 (d, J=2.8 Hz, 1H), 8.24 (s, 1H), 7.81-7.78 (m, 1H), 7.39 (t, J=7.6 Hz, 1H), 7.23-7.22 (m, 1H), 7.11 (d, J=2.8 Hz, 1H), 6.93 (s, 1H), 6.40 (s, 1H), 3.86 (s, 6H), 3.62 (s, 2H), 2.93-2.90 (m, 2H), 2.40 (s, 3H), 2.20 (s, 6H), 2.03-1.98 (m, 3H), 1.73-1.70 (m, 2H), 1.43-1.28 (m, 2H).

### Example 38, 39 and 40

The compounds of Example 38, 39 and 40 may be prepared according to the following synthetic scheme:

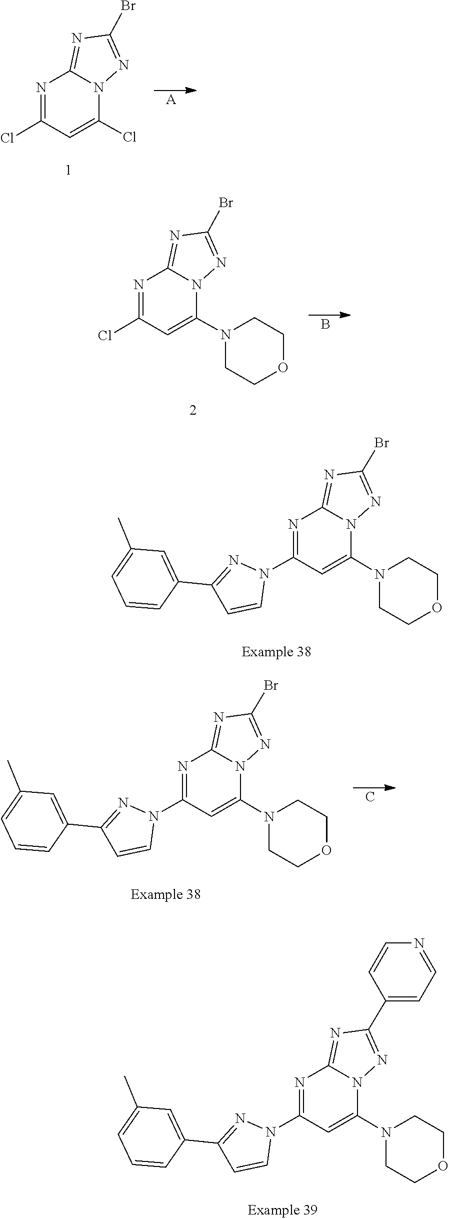

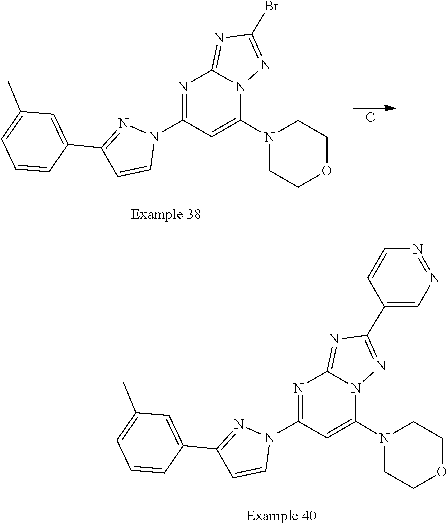

### Step A

To a stirred solution of 2-bromo-5,7-dichloro-[1,2,4]triazolo[1,5-a]pyrimidine (1) (500 mg, 1.87 mmol, 1 eq.) in DCM (10 mL) was added morpholine (195 mg, 2.24 mmol, 1.2 eq) at 0° C. The reaction mixture was allowed to warm to 25° C. and it was stirred for 16 h. The progress of the reaction was monitored by TLC and after completion, the reaction mass was evaporated to dryness to provide crude product (700 mg). The crude product was purified through silica gel column (60-120) chromatography using 70-80% EtOAc in hexane as an eluent to afford 4-(2-bromo-5-chloro-[1,2,4]triazolo[1,5-a]pyrimidin-7-yl)morpholine (2) as a white solid (250 mg, 42%).

### Step B: 4-(2-bromo-5-(3-(m-tolyl)-1H-pyrazol-1-yl)-[1,2,4]triazolo[1,5-a]pyrimidin-7-yl)morpholine (Example 38)

In a 30 mL microwave vial, sodium hydride (50 mg, 1.26 mmol, 2 eq) was added to a solution of (2) (200 mg, 0.63 mmol, 1 eq.) in THF (5 mL). The reaction mixture was stirred for 15 min and then 3-(m-tolyl)-1H-pyrazole (100 mg, 0.63 mmol, 1 eq) was added. Then the reaction mixture was irradiated in a microwave at 100° C. for 1 h. The progress of the reaction was monitored by TLC and after completion, the reaction mixture was poured into ice cold water (20 mL). It was then extracted with EtOAc (3×20 mL). Finally, the combined organic layer was washed with brine solution (10 mL) and dried over anhydrous Na₂SO₄, filtered and evaporated under reduced pressure to obtain the crude product as a brown solid (280 mg). The crude product was purified by silica gel (60-120) column chromatography using 5-10% MeOH in DCM as an eluent to afford the product (Example 38) as a white solid (100 mg, 36%). Mass (m/z): 440.1/442.1 [M+H]⁺. LCMS purity 99%. ¹H-NMR (400 MHz, DMSO-d₆): 8.74 (d, J=2.8 Hz, 1H), 7.83-7.81 (m, 1H), 7.40 (t, J=7.6 Hz, 1H), 7.25-7.23 (m, 1H), 7.18 (d, J=2.8 Hz, 1H), 7.15 (s, 1H), 3.94-3.92 (m, 4H), 3.86-3.83 (m, 4H), 2.40 (s, 3H).

### Step C: 4-(2-(pyridin-4-yl)-5-(3-(m-tolyl)-1H-pyrazol-1-yl)-[1,2,4]triazolo[1,5-a]pyrimidin-7-yl)morpholine (Example 39)

To a stirred solution of Example 38 (30 mg, 0.06 mmol, 1 eq.), and 4-(4,4,5,5-tetramethyl-1,3,2-dioxaborolan-2-yl)pyridine (28 mg, 0.13 mmol, 2 eq) in 1,4-dioxane/water (4:1) (5 mL) was added K₃PO₄ (43 mg, 0.2 mmol, 3 eq). The reaction mixture was degassed with argon for 15 min and then Pd2(dba)₃ (5 mg, 0.006 mmol, 0.1 eq), and tri-tert-butyl phosphonium tetrafluoroborate (2 mg, 0.006 mmol, 0.1 eq) were added, and the mixture was further degassed for another 10 min. The reaction mixture was irradiated in a microwave at 100° C. for 2 h. Progress of the reaction was monitored by TLC and after completion, it was cooled to room temperature and diluted with EtOAc (10 mL). The reaction mixture was filtered through celite and washed with EtOAc (10 mL), and evaporated to provide a crude product (50 mg) as black sticky compound. The crude product was purified through preparative HPLC (Method-B: Kinetex, EVO, C18, 250×21.2 mm, 5 μm; Mobile phase: [ACN: 0.1% of Formic acid in Water]; time/B %: 0/30, 10/50, 25/85), and after lyophilization afforded the product (Example 39) as an off-white solid (11 mg, 37%). Mass (m/z): 439.1 [M+H]⁺. LCMS purity 99%. ¹H-NMR (400 MHz, DMSO-d₆): 8.80-8.78 (m, 3H), 8.13-8.12 (m, 2H), 7.85-7.83 (m, 2H), 7.41 (t, J=7.6 Hz, 1H), 7.25-7.23 (m, 1H), 7.20-7.19 (m, 2H), 4.05-4.03 (m, 4H), 3.92-3.91 (m, 4H), 2.41 (s, 3H).

### Step C: 4-(2-(pyridazin-4-yl)-5-(3-(m-tolyl)-1H-pyrazol-1-yl)-[1,2,4]triazolo[1,5-a]pyrimidin-7-yl)morpholine (Example 40)

To a stirred solution of Example 38 (70 mg, 0.15 mmol, 1 eq.), and 4-(tributylstannyl)pyridazine (58 mg, 0.15 mmol, 1 eq) in 1,4-dioxane (5 mL) was added CsF (48 mg, 0.3 mmol, 2 eq) in a sealed tube. The reaction mixture was degassed with argon for 15 min then PdP(t-Bu)₃ (4 mg, 0.007 mmol, 0.05 eq) was added, and the reaction mixture was degassed for another 10 min. The reaction mixture was stirred in a sealed tube at 100° C. for 16 h and the progress of the reaction was monitored by TLC. After completion, it was cooled to room temperature and diluted with EtOAc (10 mL). The reaction mixture was filtered through celite and the filtrate evaporated to provide a crude product (80 mg) as black sticky compound. The crude product was purified by silica gel column (60-120) column chromatography using 0-5% MeOH in DCM as an eluent to afford the product (Example 40) as a white solid (6 mg, 8%). Mass (m/z): 440.3 [M+H]⁺. LCMS purity 93%. ¹H-NMR (400 MHz, DMSO-d₆): 9.96 (s, 1H), 9.48 (d, J=5.2 Hz, 1H), 8.80 (d, J=2.4 Hz, 1H), 8.36-8.35 (m, 1H), 7.85-7.83 (m, 2H), 7.41 (t, J=7.6 Hz, 1H), 7.25-7.21 (m, 3H), 4.06-4.00 (m, 4H), 3.92-3.91 (m, 4H), 2.41 (s, 3H)

Additional compounds within the scope of the present disclosure may be made according to the aforementioned procedures using modifications to reagents and conditions. Other general synthetic schemes which may be employed include the following:

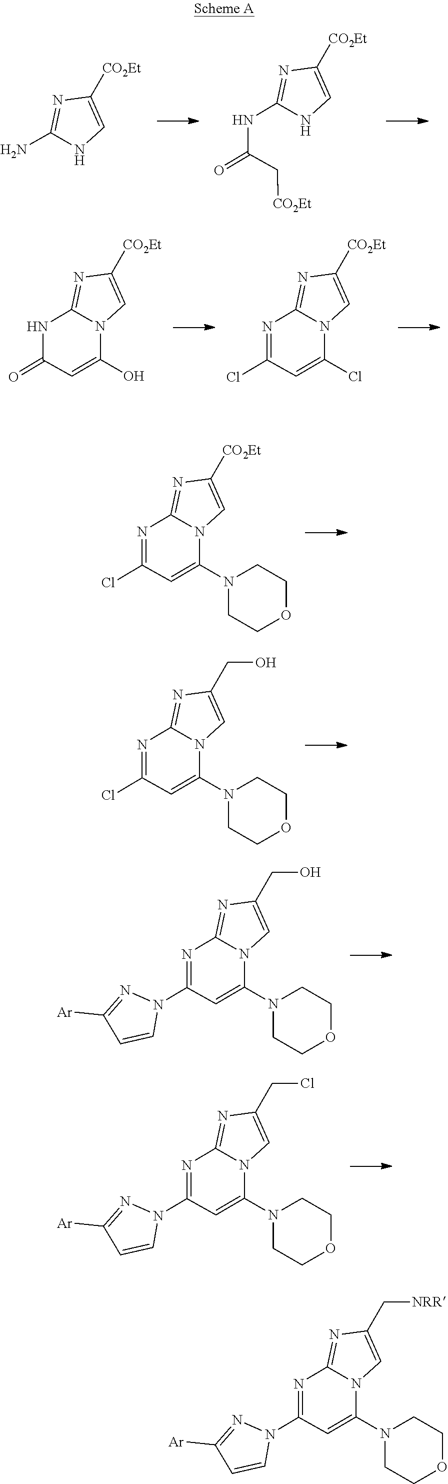

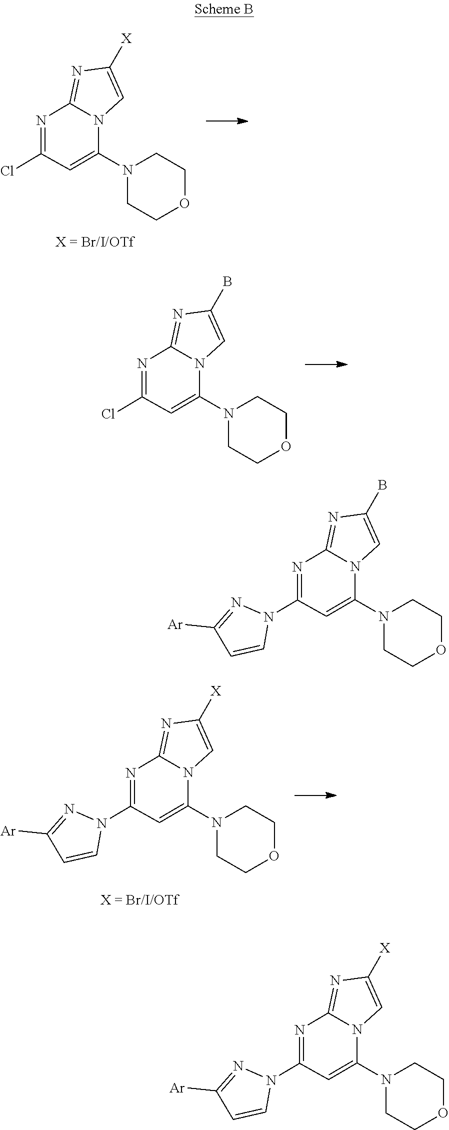

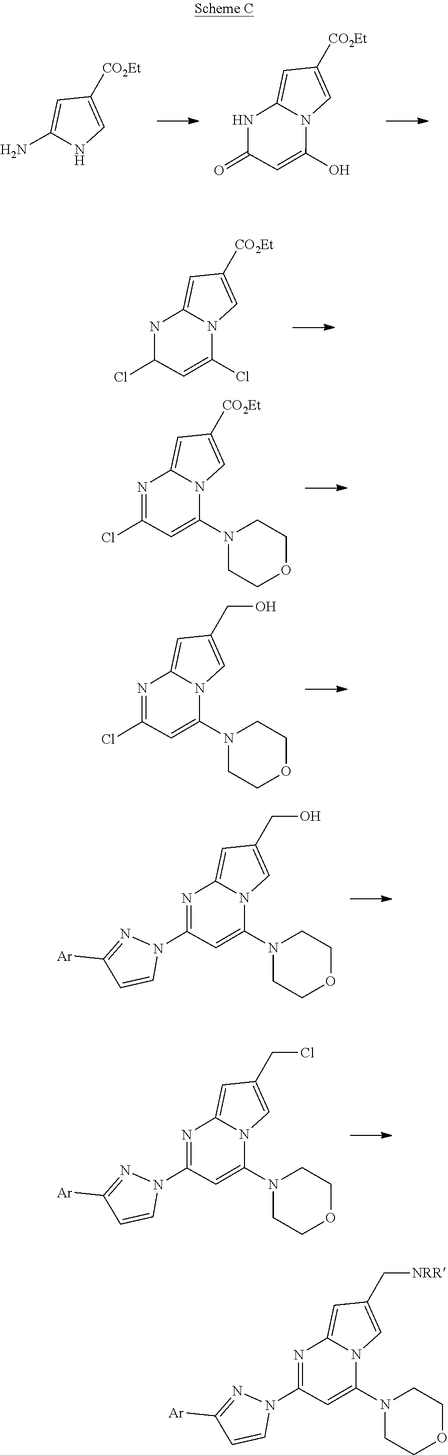

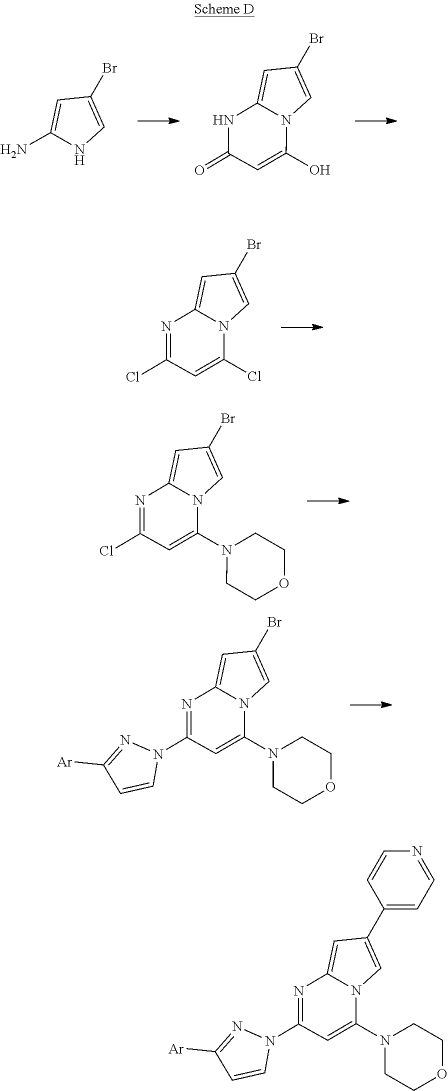

Additional synthetic procedures for preparing analogous and related compounds may also be found in the following publications: US2020/0079780, US2013/0273037, WO2005/026126, WO2010/074284, WO2013/027794, WO 2016/157091, JP2000044564A, JP2000038388A, JP2000001488A, and Richardson et al., Bioorg. & Med. Chem. Lett. 16:1353-57 (2006).

Additional compounds are provided in the following table (Table 1):

TABLE 1

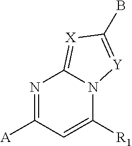

| Ex. | X | Y | A | R₁ | B |
|---|---|---|---|---|---|
| 38 | —CH— | —N— | 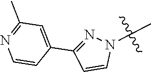 [SMILES: *n1ccc(-c2ccnc(C)c2)n1] | 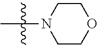 [SMILES: *N1CCOCC1] | 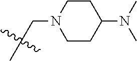 [SMILES: *CN1CCC(N(C)C)CC1] |
| 39 | —CH— | —N— | 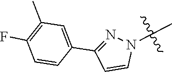 [SMILES: *n1ccc(-c2ccc(F)c(C)c2)n1] | 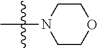 [SMILES: *N1CCOCC1] | 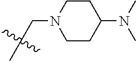 [SMILES: *CN1CCC(N(C)C)CC1] |
| 40 | —CH— | —N— | 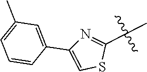 [SMILES: *c1nc(-c2cccc(C)c2)cs1] | 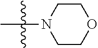 [SMILES: *N1CCOCC1] | 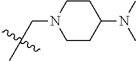 [SMILES: *CN1CCC(N(C)C)CC1] |
| 41 | —CH— | —N— | 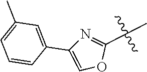 [SMILES: *c1nc(-c2cccc(C)c2)co1] | 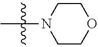 [SMILES: *N1CCOCC1] | 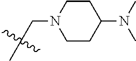 [SMILES: *CN1CCC(N(C)C)CC1] |
| 42 | —CH— | —N— | 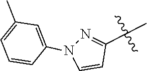 [SMILES: *c1ccn(-c2cccc(C)c2)n1] | 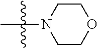 [SMILES: *N1CCOCC1] | 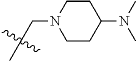 [SMILES: *CN1CCC(N(C)C)CC1] |
| 43 | —CH— | —N— | 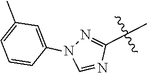 [SMILES: *c1ncn(-c2cccc(C)c2)n1] | 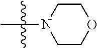 [SMILES: *N1CCOCC1] | 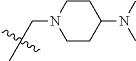 [SMILES: *CN1CCC(N(C)C)CC1] |
| 44 | —CH— | —N— | 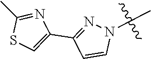 [SMILES: *n1ccc(-c2csc(C)n2)n1] | 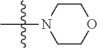 [SMILES: *N1CCOCC1] | 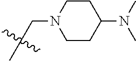 [SMILES: *CN1CCC(N(C)C)CC1] |
| 45 | —CH— | —N— | 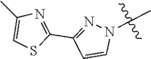 [SMILES: *n1ccc(-c2nc(C)cs2)n1] | 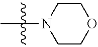 [SMILES: *N1CCOCC1] | 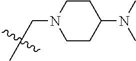 [SMILES: *CN1CCC(N(C)C)CC1] |
| 46 | —CH— | —N— | 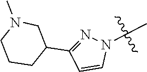 [SMILES: *n1ccc(C2CCCN(C)C2)n1] | 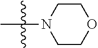 [SMILES: *N1CCOCC1] | 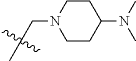 [SMILES: *CN1CCC(N(C)C)CC1] |
| 47 | —CH— | —N— | 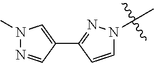 [SMILES: *n1ccc(-c2cnn(C)c2)n1] | 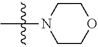 [SMILES: *N1CCOCC1] | 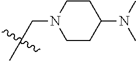 [SMILES: *CN1CCC(N(C)C)CC1] |
| 48 | —CH— | —N— | 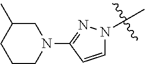 [SMILES: *n1ccc(N2CCCC(C)C2)n1] | 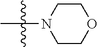 [SMILES: *N1CCOCC1] | 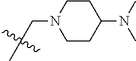 [SMILES: *CN1CCC(N(C)C)CC1] |
| 49 | —CH— | —N— | 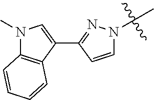 [SMILES: *n1ccc(-c2cn(C)c3ccccc23)n1] | 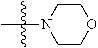 [SMILES: *N1CCOCC1] | 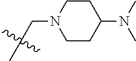 [SMILES: *CN1CCC(N(C)C)CC1] |
| 50 | —CH— | —N— | 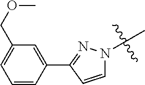 [SMILES: *n1ccc(-c2cccc(COC)c2)n1] | 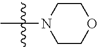 [SMILES: *N1CCOCC1] | 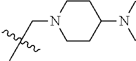 [SMILES: *CN1CCC(N(C)C)CC1] |
| 51 | —CH— | —N— | 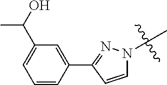 [SMILES: *n1ccc(-c2cccc(C(C)O)c2)n1] | 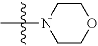 [SMILES: *N1CCOCC1] | 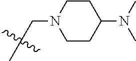 [SMILES: *CN1CCC(N(C)C)CC1] |
| 52 | —CH— | —N— | 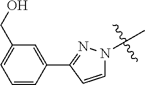 [SMILES: *n1ccc(-c2cccc(CO)c2)n1] | 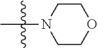 [SMILES: *N1CCOCC1] | 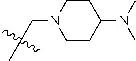 [SMILES: *CN1CCC(N(C)C)CC1] |
| 53 | —CH— | —N— | 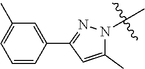 [SMILES: *n1nc(-c2cccc(C)c2)cc1C] | 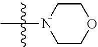 [SMILES: *N1CCOCC1] | 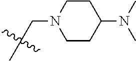 [SMILES: *CN1CCC(N(C)C)CC1] |
| 54 | —CH— | —N— | 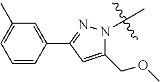 [SMILES: *n1nc(-c2cccc(C)c2)cc1COC] | 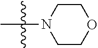 [SMILES: *N1CCOCC1] | 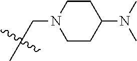 [SMILES: *CN1CCC(N(C)C)CC1] |
| 55 | —CH— | —N— | 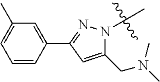 [SMILES: *n1nc(-c2cccc(C)c2)cc1CN(C)C] | 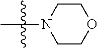 [SMILES: *N1CCOCC1] | 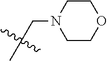 [SMILES: *CN1CCOCC1] |
| 56 | —CH— | —N— | 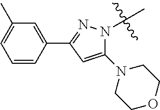 [SMILES: *n1nc(-c2cccc(C)c2)cc1N1CCOCC1] | 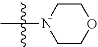 [SMILES: *N1CCOCC1] | 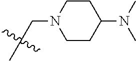 [SMILES: *CN1CCC(N(C)C)CC1] |
| 57 | —CH— | —N— | 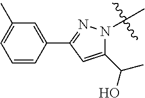 [SMILES: *n1nc(-c2cccc(C)c2)cc1C(C)O] | 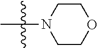 [SMILES: *N1CCOCC1] | 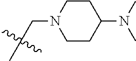 [SMILES: *CN1CCC(N(C)C)CC1] |
| 58 | —CH— | —N— | 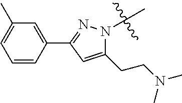 [SMILES: *n1nc(-c2cccc(C)c2)cc1CCN(C)C] | 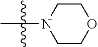 [SMILES: *N1CCOCC1] | 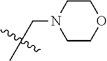 [SMILES: *CN1CCOCC1] |
| 59 | —CH— | —N— | 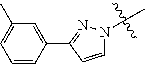 [SMILES: *n1ccc(-c2cccc(C)c2)n1] | 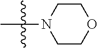 [SMILES: *N1CCOCC1] | 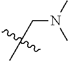 [SMILES: *CN(C)C] |
| 60 | —CH— | —N— | 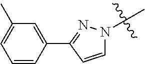 [SMILES: *n1ccc(-c2cccc(C)c2)n1] | 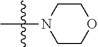 [SMILES: *N1CCOCC1] | 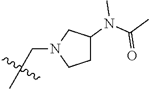 [SMILES: *CN1CCC(N(C)C(C)=O)C1] |
| 61 | —CH— | —N— | 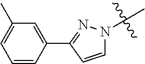 [SMILES: *n1ccc(-c2cccc(C)c2)n1] | 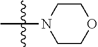 [SMILES: *N1CCOCC1] | 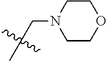 [SMILES: *CN1CCOCC1] |
| 62 | —CH— | —N— | 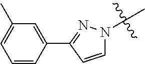 [SMILES: *n1ccc(-c2cccc(C)c2)n1] | 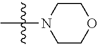 [SMILES: *N1CCOCC1] | 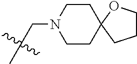 [SMILES: *CN1CCC2(CCCO2)CC1] |
| 63 | —CH— | —N— | 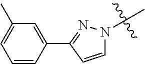 [SMILES: *n1ccc(-c2cccc(C)c2)n1] | 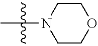 [SMILES: *N1CCOCC1] | 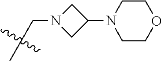 [SMILES: *CN1CC(N2CCOCC2)C1] |
| 64 | —CH— | —N— | 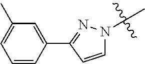 [SMILES: *n1ccc(-c2cccc(C)c2)n1] | 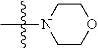 [SMILES: *N1CCOCC1] | 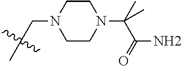 [SMILES: *CN1CCN(C(C)(C)C(N)=O)CC1] |
| 65 | —CH— | —N— | 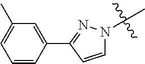 [SMILES: *n1ccc(-c2cccc(C)c2)n1] | 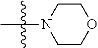 [SMILES: *N1CCOCC1] | 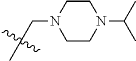 [SMILES: *CN1CCN(C(C)C)CC1] |
| 66 | —CH— | —N— | 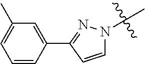 [SMILES: *n1ccc(-c2cccc(C)c2)n1] | 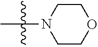 [SMILES: *N1CCOCC1] | 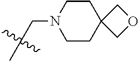 [SMILES: *CN1CCC2(CC1)COC2] |
| 67 | —CH— | —N— | 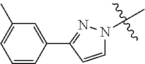 [SMILES: *n1ccc(-c2cccc(C)c2)n1] | 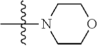 [SMILES: *N1CCOCC1] | 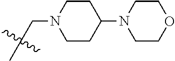 [SMILES: *CN1CCC(N2CCOCC2)CC1] |
| 68 | —CH— | —N— | 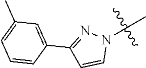 [SMILES: *n1ccc(-c2cccc(C)c2)n1] | 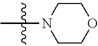 [SMILES: *N1CCOCC1] | 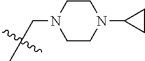 [SMILES: *CN1CCN(C2CC2)CC1] |
| 69 | —CH— | —N— | 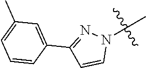 [SMILES: *n1ccc(-c2cccc(C)c2)n1] | 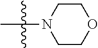 [SMILES: *N1CCOCC1] | 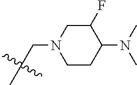 [SMILES: *CN1CCC(N(C)C)C(F)C1] |
| 70 | —CH— | —N— | 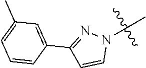 [SMILES: *n1ccc(-c2cccc(C)c2)n1] | 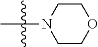 [SMILES: *N1CCOCC1] | 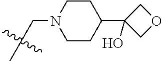 [SMILES: *CN1CCC(C2(O)COC2)CC1] |
| 71 | —CH— | —N— | 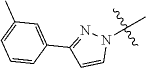 [SMILES: *n1ccc(-c2cccc(C)c2)n1] | 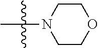 [SMILES: *N1CCOCC1] | 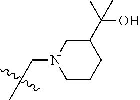 [SMILES: *CN1CCCC(C(C)(C)O)C1] |
| 72 | —CH— | —N— | 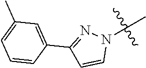 [SMILES: *n1ccc(-c2cccc(C)c2)n1] | 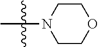 [SMILES: *N1CCOCC1] | 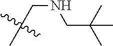 [SMILES: *CNCC(C)(C)C] |
| 73 | —CH— | —N— | 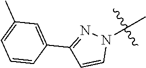 [SMILES: *n1ccc(-c2cccc(C)c2)n1] | 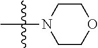 [SMILES: *N1CCOCC1] | 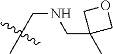 [SMILES: *CNCC1(C)COC1] |
| 74 | —CH— | —N— | 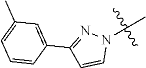 [SMILES: *n1ccc(-c2cccc(C)c2)n1] | 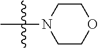 [SMILES: *N1CCOCC1] | 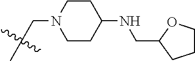 [SMILES: *CN1CCC(NCC2CCCO2)CC1] |
| 75 | —CH— | —N— | 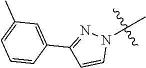 [SMILES: *n1ccc(-c2cccc(C)c2)n1] | 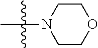 [SMILES: *N1CCOCC1] | 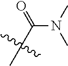 [SMILES: *C(=O)N(C)C] |
| 76 | —CH— | —N— | 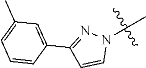 [SMILES: *n1ccc(-c2cccc(C)c2)n1] | 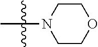 [SMILES: *N1CCOCC1] | 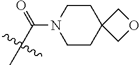 [SMILES: *C(=O)N1CCC2(CC1)COC2] |
| 77 | —CH— | —N— | 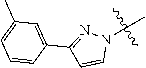 [SMILES: *n1ccc(-c2cccc(C)c2)n1] | 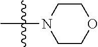 [SMILES: *N1CCOCC1] | 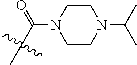 [SMILES: *C(=O)N1CCN(C(C)C)CC1] |
| 78 | —CH— | —N— | 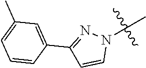 [SMILES: *n1ccc(-c2cccc(C)c2)n1] | 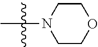 [SMILES: *N1CCOCC1] | 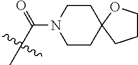 [SMILES: *C(=O)N1CCC2(CCCO2)CC1] |
| 79 | —CH— | —N— | 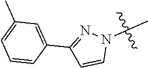 [SMILES: *n1ccc(-c2cccc(C)c2)n1] | 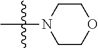 [SMILES: *N1CCOCC1] | 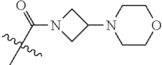 [SMILES: *C(=O)N1CC(N2CCOCC2)C1] |
| 80 | —CH— | —N— | 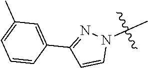 [SMILES: *n1ccc(-c2cccc(C)c2)n1] | 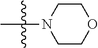 [SMILES: *N1CCOCC1] | 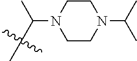 [SMILES: *C(C)N1CCN(C(C)C)CC1] |
| 81 | —CH— | —N— | 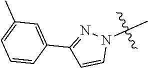 [SMILES: *n1ccc(-c2cccc(C)c2)n1] | 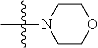 [SMILES: *N1CCOCC1] | 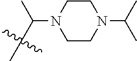 [SMILES: *C(C)N1CCN(C(C)C)CC1] |
| 82 | —CH— | —N— | 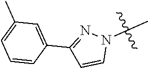 [SMILES: *n1ccc(-c2cccc(C)c2)n1] | 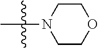 [SMILES: *N1CCOCC1] | 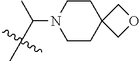 [SMILES: *C(C)N1CCC2(CC1)COC2] |
| 83 | —CH— | —N— | 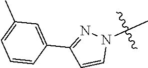 [SMILES: *n1ccc(-c2cccc(C)c2)n1] | 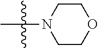 [SMILES: *N1CCOCC1] | 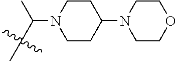 [SMILES: *C(C)N1CCC(N2CCOCC2)CC1] |
| 84 | —CH— | —N— | 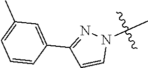 [SMILES: *n1ccc(-c2cccc(C)c2)n1] | 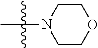 [SMILES: *N1CCOCC1] | 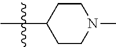 [SMILES: *C1CCN(C)CC1] |
| 85 | —CH— | —N— | 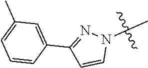 [SMILES: *n1ccc(-c2cccc(C)c2)n1] | 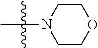 [SMILES: *N1CCOCC1] | 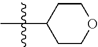 [SMILES: *C1CCOCC1] |
| 86 | —CH— | —N— | 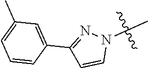 [SMILES: *n1ccc(-c2cccc(C)c2)n1] | 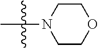 [SMILES: *N1CCOCC1] | 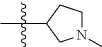 [SMILES: *C1CCN(C)C1] |
| 87 | —CH— | —N— | 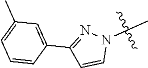 [SMILES: *n1ccc(-c2cccc(C)c2)n1] | 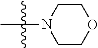 [SMILES: *N1CCOCC1] | 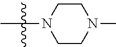 [SMILES: *N1CCN(C)CC1] |
| 88 | —CH— | —N— | 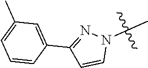 [SMILES: *n1ccc(-c2cccc(C)c2)n1] | 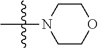 [SMILES: *N1CCOCC1] | 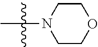 [SMILES: *N1CCOCC1] |
| 89 | —CH— | —N— | 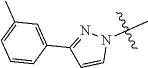 [SMILES: *n1ccc(-c2cccc(C)c2)n1] | 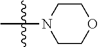 [SMILES: *N1CCOCC1] | 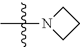 [SMILES: *N1CCC1] |
| 90 | —CH— | —N— | 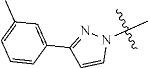 [SMILES: *n1ccc(-c2cccc(C)c2)n1] | 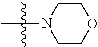 [SMILES: *N1CCOCC1] | 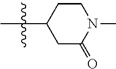 [SMILES: *C1CCN(C)C(=O)C1] |
| 91 | —CH— | —N— | 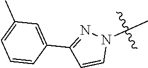 [SMILES: *n1ccc(-c2cccc(C)c2)n1] | 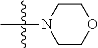 [SMILES: *N1CCOCC1] | 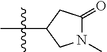 [SMILES: *C1CC(=O)N(C)C1] |
| 92 | —CH— | —N— | 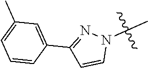 [SMILES: *n1ccc(-c2cccc(C)c2)n1] | 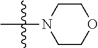 [SMILES: *N1CCOCC1] | 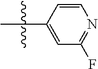 [SMILES: *c1ccnc(F)c1] |
| 93 | —CH— | —N— | 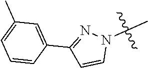 [SMILES: *n1ccc(-c2cccc(C)c2)n1] | 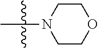 [SMILES: *N1CCOCC1] | 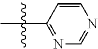 [SMILES: *c1ccncn1] |
| 94 | —CH— | —N— | 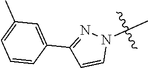 [SMILES: *n1ccc(-c2cccc(C)c2)n1] | 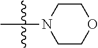 [SMILES: *N1CCOCC1] | 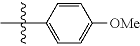 [SMILES: *c1ccc(OC)cc1] |
| 95 | —CH— | —N— | 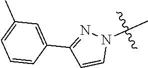 [SMILES: *n1ccc(-c2cccc(C)c2)n1] | 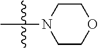 [SMILES: *N1CCOCC1] | 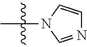 [SMILES: *n1ccnc1] |
| 96 | —CH— | —N— | 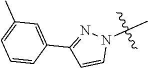 [SMILES: *n1ccc(-c2cccc(C)c2)n1] | 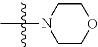 [SMILES: *N1CCOCC1] | 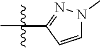 [SMILES: *c1ccn(C)n1] |
| 97 | —CH— | —N— | 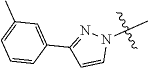 [SMILES: *n1ccc(-c2cccc(C)c2)n1] | 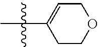 [SMILES: *C1=CCOCC1] | 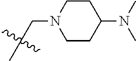 [SMILES: *CN1CCC(N(C)C)CC1] |
| 98 | —CH— | —N— | 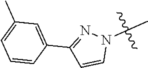 [SMILES: *n1ccc(-c2cccc(C)c2)n1] | 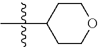 [SMILES: *C1CCOCC1] | 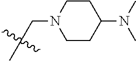 [SMILES: *CN1CCC(N(C)C)CC1] |
| 99 | —CH— | —N— | 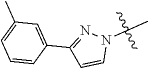 [SMILES: *n1ccc(-c2cccc(C)c2)n1] | 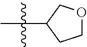 [SMILES: *C1CCOC1] | 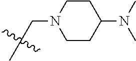 [SMILES: *CN1CCC(N(C)C)CC1] |
| 100 | —CH— | —N— | 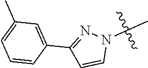 [SMILES: *n1ccc(-c2cccc(C)c2)n1] | 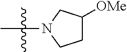 [SMILES: *N1CCC(OC)C1] | 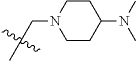 [SMILES: *CN1CCC(N(C)C)CC1] |
| 101 | —CH— | —N— | 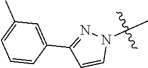 [SMILES: *n1ccc(-c2cccc(C)c2)n1] | 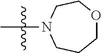 [SMILES: *N1CCCOCC1] | 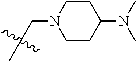 [SMILES: *CN1CCC(N(C)C)CC1] |
| 102 | —CH— | —N— | 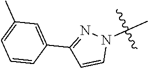 [SMILES: *n1ccc(-c2cccc(C)c2)n1] | 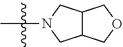 [SMILES: *N1CC2COCC2C1] | 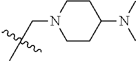 [SMILES: *CN1CCC(N(C)C)CC1] |
| 103 | —CH— | —N— | 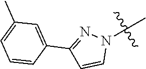 [SMILES: *n1ccc(-c2cccc(C)c2)n1] | 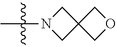 [SMILES: *N1CC2(COC2)C1] | 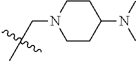 [SMILES: *CN1CCC(N(C)C)CC1] |
| 104 | —CH— | —N— | 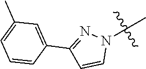 [SMILES: *n1ccc(-c2cccc(C)c2)n1] | 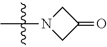 [SMILES: *N1CC(=O)C1] | 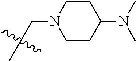 [SMILES: *CN1CCC(N(C)C)CC1] |
| 105 | —CH— | —N— | 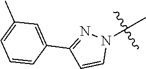 [SMILES: *n1ccc(-c2cccc(C)c2)n1] | 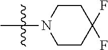 [SMILES: *N1CCC(F)(F)CC1] | 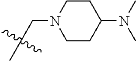 [SMILES: *CN1CCC(N(C)C)CC1] |
| 106 | —CH— | —N— | 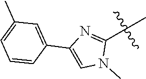 [SMILES: *c1nc(-c2cccc(C)c2)cn1C] | 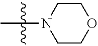 [SMILES: *N1CCOCC1] | 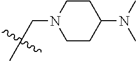 [SMILES: *CN1CCC(N(C)C)CC1] |
| 107 | —CH— | —N— | 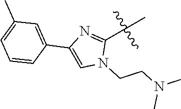 [SMILES: *c1nc(-c2cccc(C)c2)cn1CCN(C)C] | 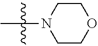 [SMILES: *N1CCOCC1] | 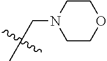 [SMILES: *CN1CCOCC1] |
| 108 | —CH— | —N— | 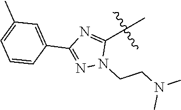 [SMILES: *c1nc(-c2cccc(C)c2)nn1CCN(C)C] | 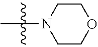 [SMILES: *N1CCOCC1] | 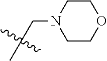 [SMILES: *CN1CCOCC1] |
| 109 | —CH— | —N— | 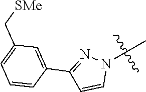 [SMILES: *n1ccc(-c2cccc(CSC)c2)n1] | 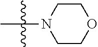 [SMILES: *N1CCOCC1] | 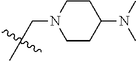 [SMILES: *CN1CCC(N(C)C)CC1] |
| 110 | —CH— | —N— | 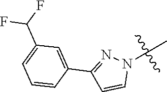 [SMILES: *n1ccc(-c2cccc(C(F)F)c2)n1] | 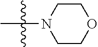 [SMILES: *N1CCOCC1] | 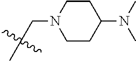 [SMILES: *CN1CCC(N(C)C)CC1] |
| 111 | —CH— | —N— | 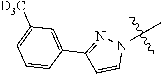 [SMILES: *n1ccc(-c2cccc(C([2H])([2H])[2H])c2)n1] | 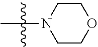 [SMILES: *N1CCOCC1] | 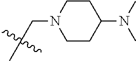 [SMILES: *CN1CCC(N(C)C)CC1] |
| 112 | —N— | —N— | 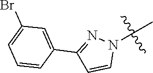 [SMILES: *n1ccc(-c2cccc(Br)c2)n1] | 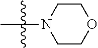 [SMILES: *N1CCOCC1] | 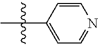 [SMILES: *c1ccncc1] |
| 113 | —N— | —N— | 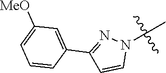 [SMILES: *n1ccc(-c2cccc(OC)c2)n1] | 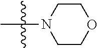 [SMILES: *N1CCOCC1] | 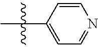 [SMILES: *c1ccncc1] |
| 114 | —N— | —N— | 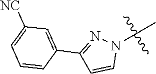 [SMILES: *n1ccc(-c2cccc(C#N)c2)n1] | 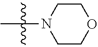 [SMILES: *N1CCOCC1] | 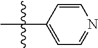 [SMILES: *c1ccncc1] |
| 115 | —N— | —N— | 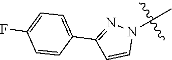 [SMILES: *n1ccc(-c2ccc(F)cc2)n1] | 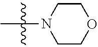 [SMILES: *N1CCOCC1] | 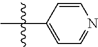 [SMILES: *c1ccncc1] |
| 116 | —N— | —N— | 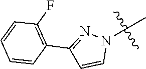 [SMILES: *n1ccc(-c2ccccc2F)n1] | 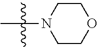 [SMILES: *N1CCOCC1] | 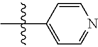 [SMILES: *c1ccncc1] |
| 117 | —N— | —N— | 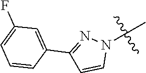 [SMILES: *n1ccc(-c2cccc(F)c2)n1] | 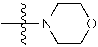 [SMILES: *N1CCOCC1] | 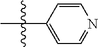 [SMILES: *c1ccncc1] |
| 118 | —N— | —N— | 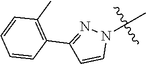 [SMILES: *n1ccc(-c2ccccc2C)n1] | 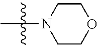 [SMILES: *N1CCOCC1] | 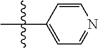 [SMILES: *c1ccncc1] |
| 119 | —N— | —N— | 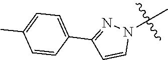 [SMILES: *n1ccc(-c2ccc(C)cc2)n1] | 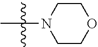 [SMILES: *N1CCOCC1] | 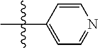 [SMILES: *c1ccncc1] |
| 120 | —N— | —N— | 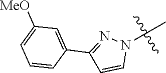 [SMILES: *n1ccc(-c2cccc(OC)c2)n1] | 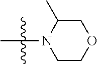 [SMILES: *N1CCOCC1C] | 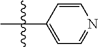 [SMILES: *c1ccncc1] |
| 121 | —N— | —N— | 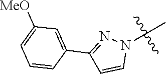 [SMILES: *n1ccc(-c2cccc(OC)c2)n1] | 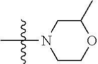 [SMILES: *N1CCOC(C)C1] | 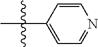 [SMILES: *c1ccncc1] |
| 122 | —N— | —N— | 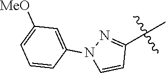 [SMILES: *c1ccn(-c2cccc(OC)c2)n1] | 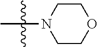 [SMILES: *N1CCOCC1] | 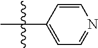 [SMILES: *c1ccncc1] |
| 123 | —N— | —N— | 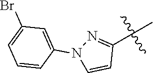 [SMILES: *c1ccn(-c2cccc(Br)c2)n1] | 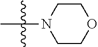 [SMILES: *N1CCOCC1] | 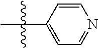 [SMILES: *c1ccncc1] |
| 124 | —N— | —N— | 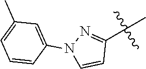 [SMILES: *c1ccn(-c2cccc(C)c2)n1] | 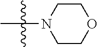 [SMILES: *N1CCOCC1] | 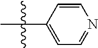 [SMILES: *c1ccncc1] |
| 125 | —N— | —N— | 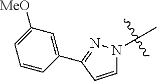 [SMILES: *n1ccc(-c2cccc(OC)c2)n1] | 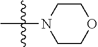 [SMILES: *N1CCOCC1] | 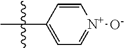 [SMILES: *c1cc[n+]([O-])cc1] |
| 126 | —N— | —N— | 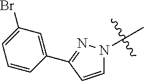 [SMILES: *n1ccc(-c2cccc(Br)c2)n1] | 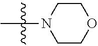 [SMILES: *N1CCOCC1] | 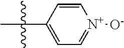 [SMILES: *c1cc[n+]([O-])cc1] |
| 127 | —N— | —N— | 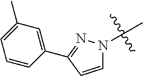 [SMILES: *n1ccc(-c2cccc(C)c2)n1] | 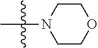 [SMILES: *N1CCOCC1] | 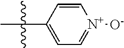 [SMILES: *c1cc[n+]([O-])cc1] |
| 128 | —N— | —N— | 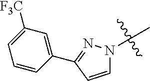 [SMILES: *n1ccc(-c2cccc(C(F)(F)F)c2)n1] | 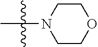 [SMILES: *N1CCOCC1] | 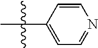 [SMILES: *c1ccncc1] |
| 129 | —N— | —N— | 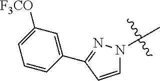 [SMILES: *n1ccc(-c2cccc(OC(F)(F)F)c2)n1] | 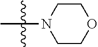 [SMILES: *N1CCOCC1] | 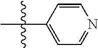 [SMILES: *c1ccncc1] |
| 130 | —N— | —N— | 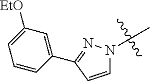 [SMILES: *n1ccc(-c2cccc(OCC)c2)n1] | 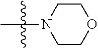 [SMILES: *N1CCOCC1] | 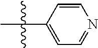 [SMILES: *c1ccncc1] |
| 131 | —N— | —N— | 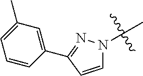 [SMILES: *n1ccc(-c2cccc(C)c2)n1] | 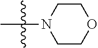 [SMILES: *N1CCOCC1] | 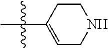 [SMILES: *C1=CCNCC1] |
| 132 | —N— | —N— | 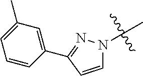 [SMILES: *n1ccc(-c2cccc(C)c2)n1] | 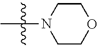 [SMILES: *N1CCOCC1] | 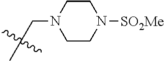 [SMILES: *CN1CCN(S(C)(=O)=O)CC1] |
| 133 | —N— | —N— | 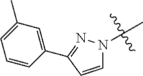 [SMILES: *n1ccc(-c2cccc(C)c2)n1] | 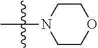 [SMILES: *N1CCOCC1] | 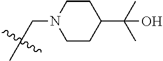 [SMILES: *CN1CCC(C(C)(C)O)CC1] |
| 134 | —N— | —N— | 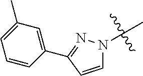 [SMILES: *n1ccc(-c2cccc(C)c2)n1] | 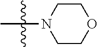 [SMILES: *N1CCOCC1] | 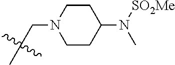 [SMILES: *CN1CCC(N(C)S(C)(=O)=O)CC1] |
| 135 | —N— | —N— | 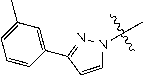 [SMILES: *n1ccc(-c2cccc(C)c2)n1] | 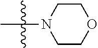 [SMILES: *N1CCOCC1] | 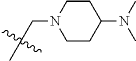 [SMILES: *CN1CCC(N(C)C)CC1] |
| 136 | —N— | —N— | 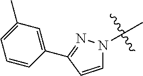 [SMILES: *n1ccc(-c2cccc(C)c2)n1] | 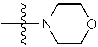 [SMILES: *N1CCOCC1] | 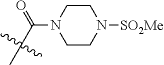 [SMILES: *C(=O)N1CCN(S(C)(=O)=O)CC1] |
| 137 | —N— | —N— | 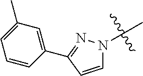 [SMILES: *n1ccc(-c2cccc(C)c2)n1] | 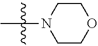 [SMILES: *N1CCOCC1] | 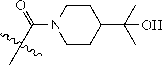 [SMILES: *C(=O)N1CCC(C(C)(C)O)CC1] |
| 138 | —N— | —N— | 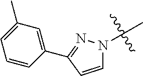 [SMILES: *n1ccc(-c2cccc(C)c2)n1] | 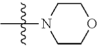 [SMILES: *N1CCOCC1] | 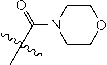 [SMILES: *C(=O)N1CCOCC1] |
| 139 | —N— | —N— | 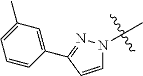 [SMILES: *n1ccc(-c2cccc(C)c2)n1] | 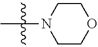 [SMILES: *N1CCOCC1] | 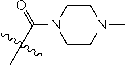 [SMILES: *C(=O)N1CCN(C)CC1] |
| 140 | —N— | —N— | 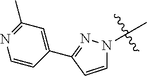 [SMILES: *n1ccc(-c2ccnc(C)c2)n1] | 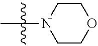 [SMILES: *N1CCOCC1] | 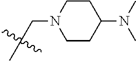 [SMILES: *CN1CCC(N(C)C)CC1] |
| 141 | —N— | —N— | 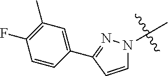 [SMILES: *n1ccc(-c2ccc(F)c(C)c2)n1] | 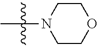 [SMILES: *N1CCOCC1] | 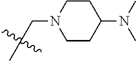 [SMILES: *CN1CCC(N(C)C)CC1] |
| 142 | —N— | —N— | 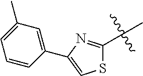 [SMILES: *c1nc(-c2cccc(C)c2)cs1] | 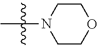 [SMILES: *N1CCOCC1] | 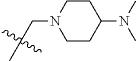 [SMILES: *CN1CCC(N(C)C)CC1] |
| 143 | —N— | —N— | 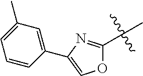 [SMILES: *c1nc(-c2cccc(C)c2)co1] | 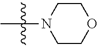 [SMILES: *N1CCOCC1] | 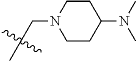 [SMILES: *CN1CCC(N(C)C)CC1] |
| 144 | —N— | —N— | 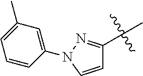 [SMILES: *c1ccn(-c2cccc(C)c2)n1] | 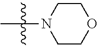 [SMILES: *N1CCOCC1] | 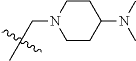 [SMILES: *CN1CCC(N(C)C)CC1] |
| 145 | —N— | —N— | 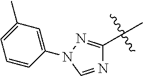 [SMILES: *c1ncn(-c2cccc(C)c2)n1] | 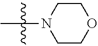 [SMILES: *N1CCOCC1] | 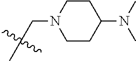 [SMILES: *CN1CCC(N(C)C)CC1] |
| 146 | —N— | —N— | 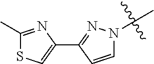 [SMILES: *n1ccc(-c2csc(C)n2)n1] | 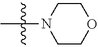 [SMILES: *N1CCOCC1] | 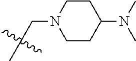 [SMILES: *CN1CCC(N(C)C)CC1] |
| 147 | —N— | —N— | 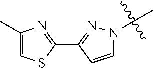 [SMILES: *n1ccc(-c2nc(C)cs2)n1] | 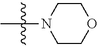 [SMILES: *N1CCOCC1] | 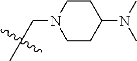 [SMILES: *CN1CCC(N(C)C)CC1] |
| 148 | —N— | —N— | 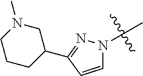 [SMILES: *n1ccc(C2CCCN(C)C2)n1] | 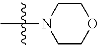 [SMILES: *N1CCOCC1] | 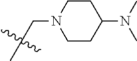 [SMILES: *CN1CCC(N(C)C)CC1] |
| 149 | —N— | —N— | 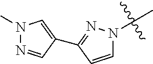 [SMILES: *n1ccc(-c2cnn(C)c2)n1] | 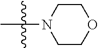 [SMILES: *N1CCOCC1] | 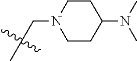 [SMILES: *CN1CCC(N(C)C)CC1] |
| 150 | —N— | —N— | 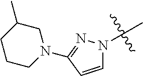 [SMILES: *n1ccc(N2CCCC(C)C2)n1] | 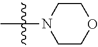 [SMILES: *N1CCOCC1] | 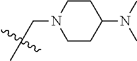 [SMILES: *CN1CCC(N(C)C)CC1] |
| 151 | —N— | —N— | 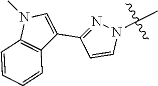 [SMILES: *n1ccc(-c2cn(C)c3ccccc23)n1] | 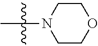 [SMILES: *N1CCOCC1] | 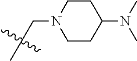 [SMILES: *CN1CCC(N(C)C)CC1] |
| 152 | —N— | —N— | 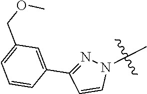 [SMILES: *n1ccc(-c2cccc(COC)c2)n1] | 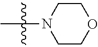 [SMILES: *N1CCOCC1] | 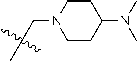 [SMILES: *CN1CCC(N(C)C)CC1] |
| 153 | —N— | —N— | 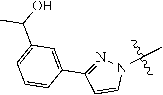 [SMILES: *n1ccc(-c2cccc(C(C)O)c2)n1] | 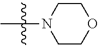 [SMILES: *N1CCOCC1] | 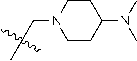 [SMILES: *CN1CCC(N(C)C)CC1] |
| 154 | —N— | —N— | 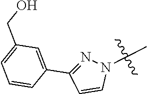 [SMILES: *n1ccc(-c2cccc(CO)c2)n1] | 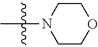 [SMILES: *N1CCOCC1] | 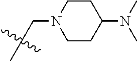 [SMILES: *CN1CCC(N(C)C)CC1] |
| 155 | —N— | —N— | 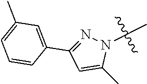 [SMILES: *n1nc(-c2cccc(C)c2)cc1C] | 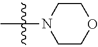 [SMILES: *N1CCOCC1] | 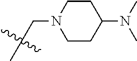 [SMILES: *CN1CCC(N(C)C)CC1] |
| 156 | —N— | —N— | 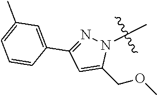 [SMILES: *n1nc(-c2cccc(C)c2)cc1COC] | 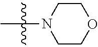 [SMILES: *N1CCOCC1] | 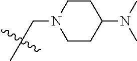 [SMILES: *CN1CCC(N(C)C)CC1] |
| 157 | —N— | —N— | 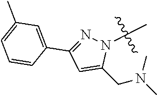 [SMILES: *n1nc(-c2cccc(C)c2)cc1CN(C)C] | 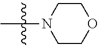 [SMILES: *N1CCOCC1] | 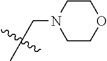 [SMILES: *CN1CCOCC1] |
| 158 | —N— | —N— | 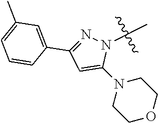 [SMILES: *n1nc(-c2cccc(C)c2)cc1N1CCOCC1] | 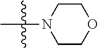 [SMILES: *N1CCOCC1] | 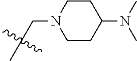 [SMILES: *CN1CCC(N(C)C)CC1] |
| 159 | —N— | —N— | 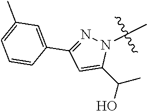 [SMILES: *n1nc(-c2cccc(C)c2)cc1C(C)O] | 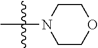 [SMILES: *N1CCOCC1] | 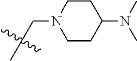 [SMILES: *CN1CCC(N(C)C)CC1] |
| 160 | —N— | —N— | 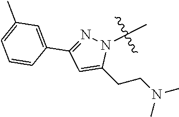 [SMILES: *n1nc(-c2cccc(C)c2)cc1CCN(C)C] | 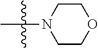 [SMILES: *N1CCOCC1] | 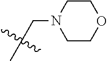 [SMILES: *CN1CCOCC1] |
| 161 | —N— | —N— | 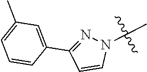 [SMILES: *n1ccc(-c2cccc(C)c2)n1] | 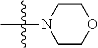 [SMILES: *N1CCOCC1] | 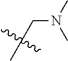 [SMILES: *CN(C)C] |
| 162 | —N— | —N— | 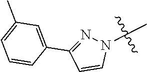 [SMILES: *n1ccc(-c2cccc(C)c2)n1] | 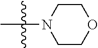 [SMILES: *N1CCOCC1] | 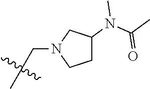 [SMILES: *CN1CCC(N(C)C(C)=O)C1] |
| 163 | —N— | —N— | 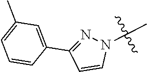 [SMILES: *n1ccc(-c2cccc(C)c2)n1] | 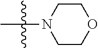 [SMILES: *N1CCOCC1] | 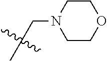 [SMILES: *CN1CCOCC1] |
| 164 | —N— | —N— | 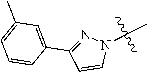 [SMILES: *n1ccc(-c2cccc(C)c2)n1] | 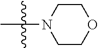 [SMILES: *N1CCOCC1] | 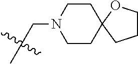 [SMILES: *CN1CCC2(CCCO2)CC1] |
| 165 | —N— | —N— | 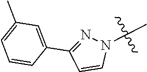 [SMILES: *n1ccc(-c2cccc(C)c2)n1] | 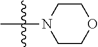 [SMILES: *N1CCOCC1] | 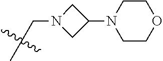 [SMILES: *CN1CC(N2CCOCC2)C1] |
| 166 | —N— | —N— | 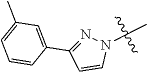 [SMILES: *n1ccc(-c2cccc(C)c2)n1] | 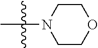 [SMILES: *N1CCOCC1] | 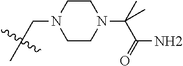 [SMILES: *CN1CCN(C(C)(C)C(N)=O)CC1] |
| 167 | —N— | —N— | 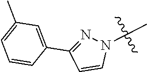 [SMILES: *n1ccc(-c2cccc(C)c2)n1] | 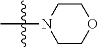 [SMILES: *N1CCOCC1] | 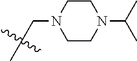 [SMILES: *CN1CCN(C(C)C)CC1] |
| 168 | —N— | —N— | 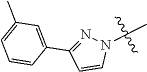 [SMILES: *n1ccc(-c2cccc(C)c2)n1] | 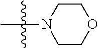 [SMILES: *N1CCOCC1] | 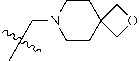 [SMILES: *CN1CCC2(CC1)COC2] |
| 169 | —N— | —N— | 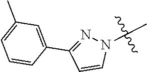 [SMILES: *n1ccc(-c2cccc(C)c2)n1] | 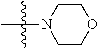 [SMILES: *N1CCOCC1] | 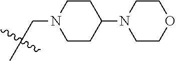 [SMILES: *CN1CCC(N2CCOCC2)CC1] |
| 170 | —N— | —N— | 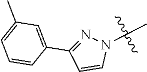 [SMILES: *n1ccc(-c2cccc(C)c2)n1] | 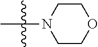 [SMILES: *N1CCOCC1] | 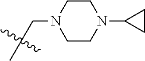 [SMILES: *CN1CCN(C2CC2)CC1] |
| 171 | —N— | —N— | 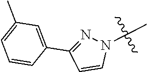 [SMILES: *n1ccc(-c2cccc(C)c2)n1] | 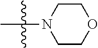 [SMILES: *N1CCOCC1] | 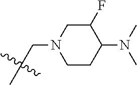 [SMILES: *CN1CCC(N(C)C)C(F)C1] |
| 172 | —N— | —N— | 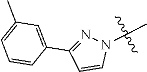 [SMILES: *n1ccc(-c2cccc(C)c2)n1] | 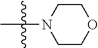 [SMILES: *N1CCOCC1] | 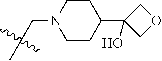 [SMILES: *CN1CCC(C2(O)COC2)CC1] |
| 173 | —N— | —N— | 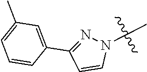 [SMILES: *n1ccc(-c2cccc(C)c2)n1] | 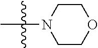 [SMILES: *N1CCOCC1] | 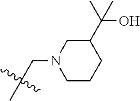 [SMILES: *CN1CCCC(C(C)(C)O)C1] |
| 174 | —N— | —N— | 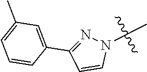 [SMILES: *n1ccc(-c2cccc(C)c2)n1] | 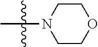 [SMILES: *N1CCOCC1] | 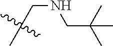 [SMILES: *CNCC(C)(C)C] |
| 175 | —N— | —N— | 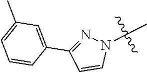 [SMILES: *n1ccc(-c2cccc(C)c2)n1] | 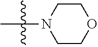 [SMILES: *N1CCOCC1] | 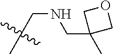 [SMILES: *CNCC1(C)COC1] |
| 176 | —N— | —N— | 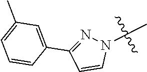 [SMILES: *n1ccc(-c2cccc(C)c2)n1] | 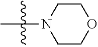 [SMILES: *N1CCOCC1] | 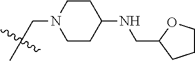 [SMILES: *CN1CCC(NCC2CCCO2)CC1] |
| 177 | —N— | —N— | 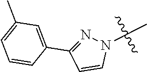 [SMILES: *n1ccc(-c2cccc(C)c2)n1] | 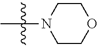 [SMILES: *N1CCOCC1] | 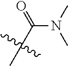 [SMILES: *C(=O)N(C)C] |
| 178 | —N— | —N— | 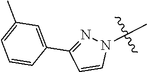 [SMILES: *n1ccc(-c2cccc(C)c2)n1] | 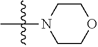 [SMILES: *N1CCOCC1] | 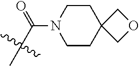 [SMILES: *C(=O)N1CCC2(CC1)COC2] |
| 179 | —N— | —N— | 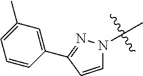 [SMILES: *n1ccc(-c2cccc(C)c2)n1] | 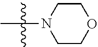 [SMILES: *N1CCOCC1] | 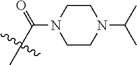 [SMILES: *C(=O)N1CCN(C(C)C)CC1] |
| 180 | —N— | —N— | 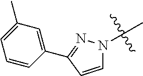 [SMILES: *n1ccc(-c2cccc(C)c2)n1] | 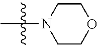 [SMILES: *N1CCOCC1] | 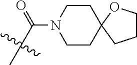 [SMILES: *C(=O)N1CCC2(CCCO2)CC1] |
| 181 | —N— | —N— | 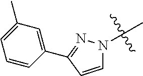 [SMILES: *n1ccc(-c2cccc(C)c2)n1] | 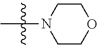 [SMILES: *N1CCOCC1] | 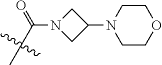 [SMILES: *C(=O)N1CC(N2CCOCC2)C1] |
| 182 | —N— | —N— | 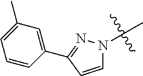 [SMILES: *n1ccc(-c2cccc(C)c2)n1] | 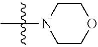 [SMILES: *N1CCOCC1] | 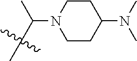 [SMILES: *C(C)N1CCC(N(C)C)CC1] |
| 183 | —N— | —N— | 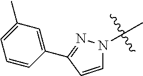 [SMILES: *n1ccc(-c2cccc(C)c2)n1] | 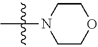 [SMILES: *N1CCOCC1] | 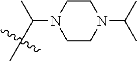 [SMILES: *C(C)N1CCN(C(C)C)CC1] |
| 184 | —N— | —N— | 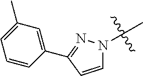 [SMILES: *n1ccc(-c2cccc(C)c2)n1] | 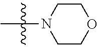 [SMILES: *N1CCOCC1] | 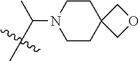 [SMILES: *C(C)N1CCC2(CC1)COC2] |
| 185 | —N— | —N— | 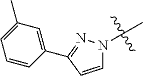 [SMILES: *n1ccc(-c2cccc(C)c2)n1] | 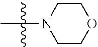 [SMILES: *N1CCOCC1] | 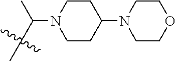 [SMILES: *C(C)N1CCC(N2CCOCC2)CC1] |
| 186 | —N— | —N— | 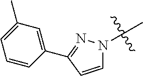 [SMILES: *n1ccc(-c2cccc(C)c2)n1] | 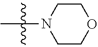 [SMILES: *N1CCOCC1] | 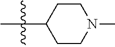 [SMILES: *C1CCN(C)CC1] |
| 187 | —N— | —N— | 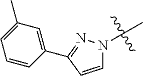 [SMILES: *n1ccc(-c2cccc(C)c2)n1] | 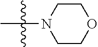 [SMILES: *N1CCOCC1] | 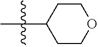 [SMILES: *C1CCOCC1] |
| 188 | —N— | —N— | 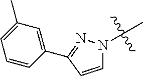 [SMILES: *n1ccc(-c2cccc(C)c2)n1] | 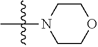 [SMILES: *N1CCOCC1] | 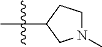 [SMILES: *C1CCN(C)C1] |
| 189 | —N— | —N— | 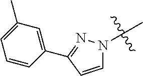 [SMILES: *n1ccc(-c2cccc(C)c2)n1] | 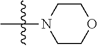 [SMILES: *N1CCOCC1] | 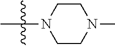 [SMILES: *N1CCN(C)CC1] |
| 190 | —N— | —N— | 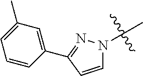 [SMILES: *n1ccc(-c2cccc(C)c2)n1] | 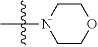 [SMILES: *N1CCOCC1] | 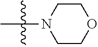 [SMILES: *N1CCOCC1] |
| 191 | —N— | —N— | 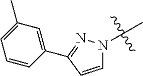 [SMILES: *n1ccc(-c2cccc(C)c2)n1] | 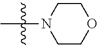 [SMILES: *N1CCOCC1] | 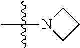 [SMILES: *N1CCC1] |
| 192 | —N— | —N— | 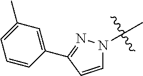 [SMILES: *n1ccc(-c2cccc(C)c2)n1] | 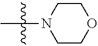 [SMILES: *N1CCOCC1] | 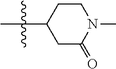 [SMILES: *C1CCN(C)C(=O)C1] |
| 193 | —N— | —N— | 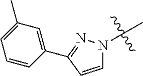 [SMILES: *n1ccc(-c2cccc(C)c2)n1] | 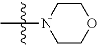 [SMILES: *N1CCOCC1] | 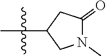 [SMILES: *C1CC(=O)N(C)C1] |
| 194 | —N— | —N— | 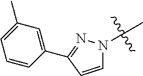 [SMILES: *n1ccc(-c2cccc(C)c2)n1] | 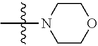 [SMILES: *N1CCOCC1] | 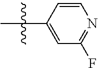 [SMILES: *c1ccnc(F)c1] |
| 195 | —N— | —N— | 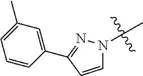 [SMILES: *n1ccc(-c2cccc(C)c2)n1] | 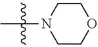 [SMILES: *N1CCOCC1] | 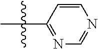 [SMILES: *c1ccncn1] |
| 196 | —N— | —N— | 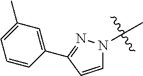 [SMILES: *n1ccc(-c2cccc(C)c2)n1] | 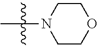 [SMILES: *N1CCOCC1] | 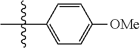 [SMILES: *c1ccc(OC)cc1] |
| 197 | —N— | —N— | 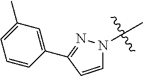 [SMILES: *n1ccc(-c2cccc(C)c2)n1] | 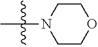 [SMILES: *N1CCOCC1] | 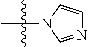 [SMILES: *n1ccnc1] |
| 198 | —N— | —N— | 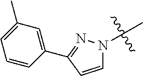 [SMILES: *n1ccc(-c2cccc(C)c2)n1] | 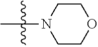 [SMILES: *N1CCOCC1] | 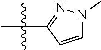 [SMILES: *c1ccn(C)n1] |
| 199 | —N— | —N— | 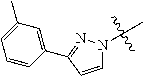 [SMILES: *n1ccc(-c2cccc(C)c2)n1] | 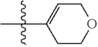 [SMILES: *C1=CCOCC1] | 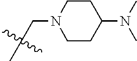 [SMILES: *CN1CCC(N(C)C)CC1] |
| 200 | —N— | —N— | 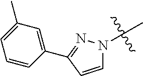 [SMILES: *n1ccc(-c2cccc(C)c2)n1] | 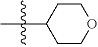 [SMILES: *C1CCOCC1] | 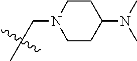 [SMILES: *CN1CCC(N(C)C)CC1] |
| 201 | —N— | —N— | 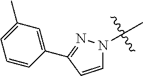 [SMILES: *n1ccc(-c2cccc(C)c2)n1] | 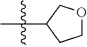 [SMILES: *C1CCOC1] | 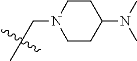 [SMILES: *CN1CCC(N(C)C)CC1] |
| 202 | —N— | —N— | 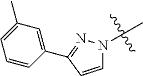 [SMILES: *n1ccc(-c2cccc(C)c2)n1] | 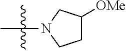 [SMILES: *N1CCC(OC)C1] | 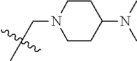 [SMILES: *CN1CCC(N(C)C)CC1] |
| 203 | —N— | —N— | 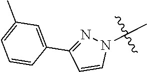 [SMILES: *n1ccc(-c2cccc(C)c2)n1] | 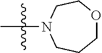 [SMILES: *N1CCCOCC1] | 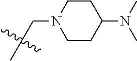 [SMILES: *CN1CCC(N(C)C)CC1] |
| 204 | —N— | —N— | 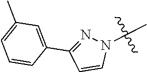 [SMILES: *n1ccc(-c2cccc(C)c2)n1] | 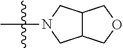 [SMILES: *N1CC2COCC2C1] | 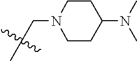 [SMILES: *CN1CCC(N(C)C)CC1] |
| 205 | —N— | —N— | 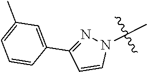 [SMILES: *n1ccc(-c2cccc(C)c2)n1] | 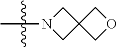 [SMILES: *N1CC2(COC2)C1] | 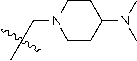 [SMILES: *CN1CCC(N(C)C)CC1] |
| 206 | —N— | —N— | 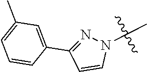 [SMILES: *n1ccc(-c2cccc(C)c2)n1] | 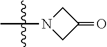 [SMILES: *N1CC(=O)C1] | 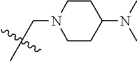 [SMILES: *CN1CCC(N(C)C)CC1] |
| 207 | —N— | —N— | 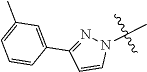 [SMILES: *n1ccc(-c2cccc(C)c2)n1] | 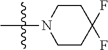 [SMILES: *N1CCC(F)(F)CC1] | 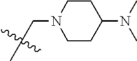 [SMILES: *CN1CCC(N(C)C)CC1] |
| 208 | —N— | —N— | 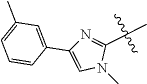 [SMILES: *c1nc(-c2cccc(C)c2)cn1C] | 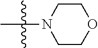 [SMILES: *N1CCOCC1] | 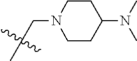 [SMILES: *CN1CCC(N(C)C)CC1] |
| 209 | —N— | —N— | 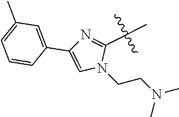 [SMILES: *c1nc(-c2cccc(C)c2)cn1CCN(C)C] | 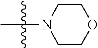 [SMILES: *N1CCOCC1] | 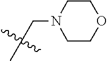 [SMILES: *CN1CCOCC1] |
| 210 | —N— | —N— | 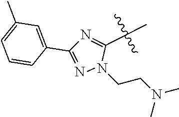 [SMILES: *c1nc(-c2cccc(C)c2)nn1CCN(C)C] | 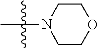 [SMILES: *N1CCOCC1] | 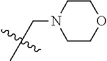 [SMILES: *CN1CCOCC1] |
| 211 | —N— | —N— | 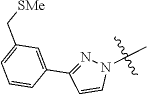 [SMILES: *n1ccc(-c2cccc(CSC)c2)n1] | 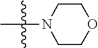 [SMILES: *N1CCOCC1] | 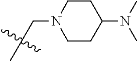 [SMILES: *CN1CCC(N(C)C)CC1] |
| 212 | —N— | —N— | 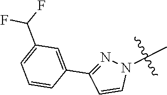 [SMILES: *n1ccc(-c2cccc(C(F)F)c2)n1] | 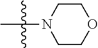 [SMILES: *N1CCOCC1] | 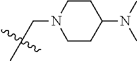 [SMILES: *CN1CCC(N(C)C)CC1] |
| 213 | —N— | —N— | 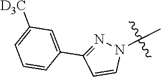 [SMILES: *n1ccc(-c2cccc(C([2H])([2H])[2H])c2)n1] | 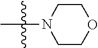 [SMILES: *N1CCOCC1] | 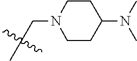 [SMILES: *CN1CCC(N(C)C)CC1] |
| 214 | —N— | —CH— | 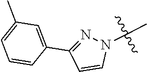 [SMILES: *n1ccc(-c2cccc(C)c2)n1] | 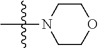 [SMILES: *N1CCOCC1] | 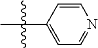 [SMILES: *c1ccncc1] |
| 215 | —N— | —CH— | 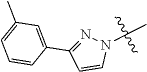 [SMILES: *n1ccc(-c2cccc(C)c2)n1] | 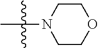 [SMILES: *N1CCOCC1] | 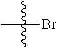 [SMILES: *Br] |
| 216 | —N— | —CH— | 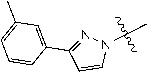 [SMILES: *n1ccc(-c2cccc(C)c2)n1] | 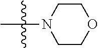 [SMILES: *N1CCOCC1] | 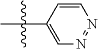 [SMILES: *c1ccnnc1] |
| 217 | —N— | —CH— | 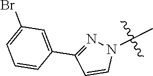 [SMILES: *n1ccc(-c2cccc(Br)c2)n1] | 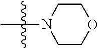 [SMILES: *N1CCOCC1] | 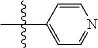 [SMILES: *c1ccncc1] |
| 218 | —N— | —CH— | 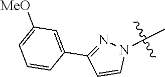 [SMILES: *n1ccc(-c2cccc(OC)c2)n1] | 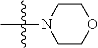 [SMILES: *N1CCOCC1] | 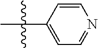 [SMILES: *c1ccncc1] |
| 219 | —N— | CH- | 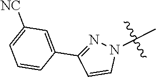 [SMILES: *n1ccc(-c2cccc(C#N)c2)n1] | 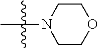 [SMILES: *N1CCOCC1] | 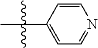 [SMILES: *c1ccncc1] |
| 220 | —N— | —CH— | 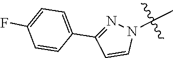 [SMILES: *n1ccc(-c2ccc(F)cc2)n1] | 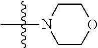 [SMILES: *N1CCOCC1] | 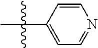 [SMILES: *c1ccncc1] |
| 221 | —N— | —CH— | 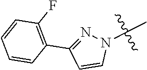 [SMILES: *n1ccc(-c2ccccc2F)n1] | 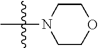 [SMILES: *N1CCOCC1] | 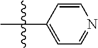 [SMILES: *c1ccncc1] |
| 222 | —N— | —CH— | 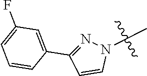 [SMILES: *n1ccc(-c2cccc(F)c2)n1] | 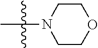 [SMILES: *N1CCOCC1] | 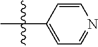 [SMILES: *c1ccncc1] |
| 223 | —N— | —CH— | 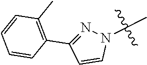 [SMILES: *n1ccc(-c2ccccc2C)n1] | 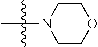 [SMILES: *N1CCOCC1] | 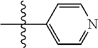 [SMILES: *c1ccncc1] |
| 224 | —N— | —CH— | 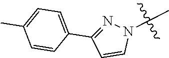 [SMILES: *n1ccc(-c2ccc(C)cc2)n1] | 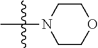 [SMILES: *N1CCOCC1] | 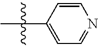 [SMILES: *c1ccncc1] |
| 225 | —N— | —CH— | 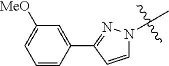 [SMILES: *n1ccc(-c2cccc(OC)c2)n1] | 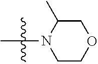 [SMILES: *N1CCOCC1C] | 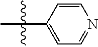 [SMILES: *c1ccncc1] |
| 226 | —N— | —CH— | 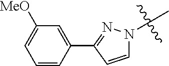 [SMILES: *n1ccc(-c2cccc(OC)c2)n1] | 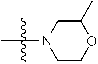 [SMILES: *N1CCOC(C)C1] | 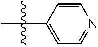 [SMILES: *c1ccncc1] |
| 227 | —N— | —CH— | 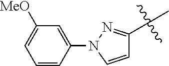 [SMILES: *c1ccn(-c2cccc(OC)c2)n1] | 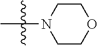 [SMILES: *N1CCOCC1] | 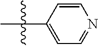 [SMILES: *c1ccncc1] |
| 228 | —N— | —CH— | 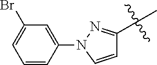 [SMILES: *c1ccn(-c2cccc(Br)c2)n1] | 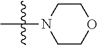 [SMILES: *N1CCOCC1] | 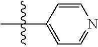 [SMILES: *c1ccncc1] |
| 229 | —N— | —CH— | 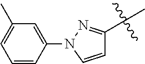 [SMILES: *c1ccn(-c2cccc(C)c2)n1] | 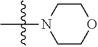 [SMILES: *N1CCOCC1] | 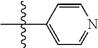 [SMILES: *c1ccncc1] |
| 230 | —N— | —CH— | 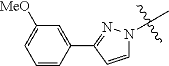 [SMILES: *n1ccc(-c2cccc(OC)c2)n1] | 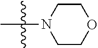 [SMILES: *N1CCOCC1] | 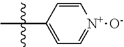 [SMILES: *c1cc[n+]([O-])cc1] |
| 231 | —N— | —CH— | 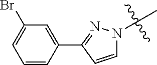 [SMILES: *n1ccc(-c2cccc(Br)c2)n1] | 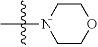 [SMILES: *N1CCOCC1] | 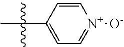 [SMILES: *c1cc[n+]([O-])cc1] |
| 232 | —N— | —CH— | 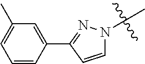 [SMILES: *n1ccc(-c2cccc(C)c2)n1] | 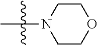 [SMILES: *N1CCOCC1] | 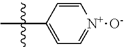 [SMILES: *c1cc[n+]([O-])cc1] |
| 233 | —N— | —CH— | 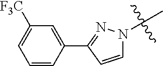 [SMILES: *n1ccc(-c2cccc(C(F)(F)F)c2)n1] | 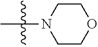 [SMILES: *N1CCOCC1] | 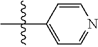 [SMILES: *c1ccncc1] |
| 234 | —N— | —CH— | 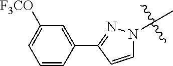 [SMILES: *n1ccc(-c2cccc(OC(F)(F)F)c2)n1] | 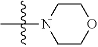 [SMILES: *N1CCOCC1] | 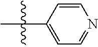 [SMILES: *c1ccncc1] |
| 235 | —N— | —CH— | 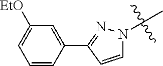 [SMILES: *n1ccc(-c2cccc(OCC)c2)n1] | 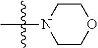 [SMILES: *N1CCOCC1] | 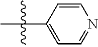 [SMILES: *c1ccncc1] |
| 236 | —N— | —CH— | 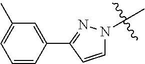 [SMILES: *n1ccc(-c2cccc(C)c2)n1] | 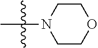 [SMILES: *N1CCOCC1] | 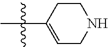 [SMILES: *C1=CCNCC1] |
| 237 | —N— | —CH— | 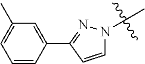 [SMILES: *n1ccc(-c2cccc(C)c2)n1] | 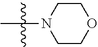 [SMILES: *N1CCOCC1] | 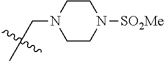 [SMILES: *CN1CCN(S(C)(=O)=O)CC1] |
| 238 | —N— | —CH— | 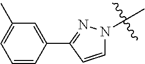 [SMILES: *n1ccc(-c2cccc(C)c2)n1] | 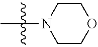 [SMILES: *N1CCOCC1] | 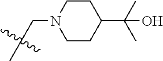 [SMILES: *CN1CCC(C(C)(C)O)CC1] |
| 239 | —N— | —CH— | 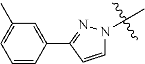 [SMILES: *n1ccc(-c2cccc(C)c2)n1] | 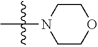 [SMILES: *N1CCOCC1] | 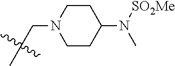 [SMILES: *CN1CCC(N(C)S(C)(=O)=O)CC1] |
| 240 | —N— | —CH— | 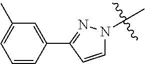 [SMILES: *n1ccc(-c2cccc(C)c2)n1] | 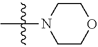 [SMILES: *N1CCOCC1] | 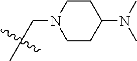 [SMILES: *CN1CCC(N(C)C)CC1] |
| 241 | —N— | —CH— | 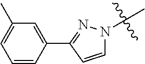 [SMILES: *n1ccc(-c2cccc(C)c2)n1] | 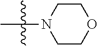 [SMILES: *N1CCOCC1] | 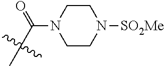 [SMILES: *C(=O)N1CCN(S(C)(=O)=O)CC1] |
| 242 | —N— | —CH— | 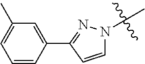 [SMILES: *n1ccc(-c2cccc(C)c2)n1] | 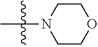 [SMILES: *N1CCOCC1] | 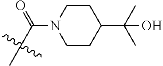 [SMILES: *C(=O)N1CCC(C(C)(C)O)CC1] |
| 243 | —N— | —CH— | 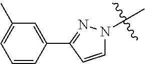 [SMILES: *n1ccc(-c2cccc(C)c2)n1] | 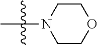 [SMILES: *N1CCOCC1] | 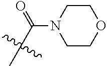 [SMILES: *C(=O)N1CCOCC1] |
| 244 | —N— | —CH— | 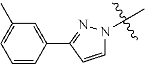 [SMILES: *n1ccc(-c2cccc(C)c2)n1] | 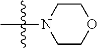 [SMILES: *N1CCOCC1] | 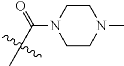 [SMILES: *C(=O)N1CCN(C)CC1] |
| 245 | —N— | —CH— | 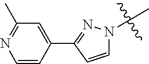 [SMILES: *n1ccc(-c2ccnc(C)c2)n1] | 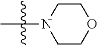 [SMILES: *N1CCOCC1] | 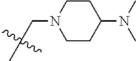 [SMILES: *CN1CCC(N(C)C)CC1] |
| 246 | —N— | —CH— | 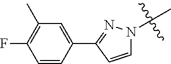 [SMILES: *n1ccc(-c2ccc(F)c(C)c2)n1] | 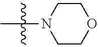 [SMILES: *N1CCOCC1] | 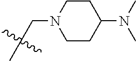 [SMILES: *CN1CCC(N(C)C)CC1] |
| 247 | —N— | —CH— | 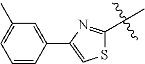 [SMILES: *c1nc(-c2cccc(C)c2)cs1] | 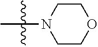 [SMILES: *N1CCOCC1] | 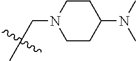 [SMILES: *CN1CCC(N(C)C)CC1] |
| 248 | —N— | —CH— | 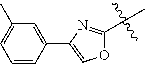 [SMILES: *c1nc(-c2cccc(C)c2)co1] | 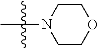 [SMILES: *N1CCOCC1] | 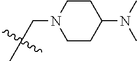 [SMILES: *CN1CCC(N(C)C)CC1] |
| 249 | —N— | —CH— | 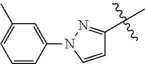 [SMILES: *c1ccn(-c2cccc(C)c2)n1] | 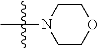 [SMILES: *N1CCOCC1] | 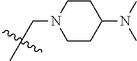 [SMILES: *CN1CCC(N(C)C)CC1] |
| 250 | —N— | —CH— | 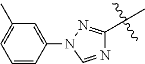 [SMILES: *c1ncn(-c2cccc(C)c2)n1] | 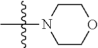 [SMILES: *N1CCOCC1] | 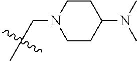 [SMILES: *CN1CCC(N(C)C)CC1] |
| 251 | —N— | —CH— | 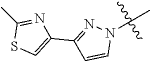 [SMILES: *n1ccc(-c2csc(C)n2)n1] | 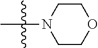 [SMILES: *N1CCOCC1] | 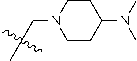 [SMILES: *CN1CCC(N(C)C)CC1] |
| 252 | —N— | —CH— | 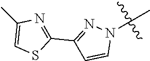 [SMILES: *n1ccc(-c2nc(C)cs2)n1] | 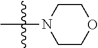 [SMILES: *N1CCOCC1] | 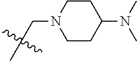 [SMILES: *CN1CCC(N(C)C)CC1] |
| 253 | —N— | CH- | 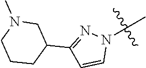 [SMILES: *n1ccc(C2CCCN(C)C2)n1] | 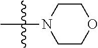 [SMILES: *N1CCOCC1] | 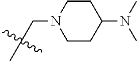 [SMILES: *CN1CCC(N(C)C)CC1] |
| 254 | —N— | —CH— | 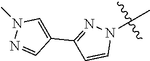 [SMILES: *n1ccc(-c2cnn(C)c2)n1] | 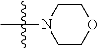 [SMILES: *N1CCOCC1] | 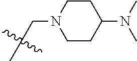 [SMILES: *CN1CCC(N(C)C)CC1] |
| 255 | —N— | —CH— | 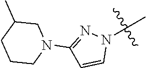 [SMILES: *n1ccc(N2CCCC(C)C2)n1] | 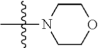 [SMILES: *N1CCOCC1] | 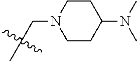 [SMILES: *CN1CCC(N(C)C)CC1] |
| 256 | —N— | —CH— | 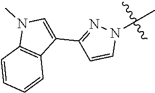 [SMILES: *n1ccc(-c2cn(C)c3ccccc23)n1] | 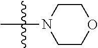 [SMILES: *N1CCOCC1] | 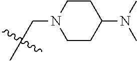 [SMILES: *CN1CCC(N(C)C)CC1] |
| 257 | —N— | —CH— | 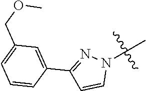 [SMILES: *n1ccc(-c2cccc(COC)c2)n1] | 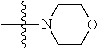 [SMILES: *N1CCOCC1] | 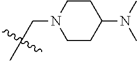 [SMILES: *CN1CCC(N(C)C)CC1] |
| 258 | —N— | —CH— | 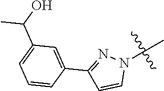 [SMILES: *n1ccc(-c2cccc(C(C)O)c2)n1] | 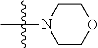 [SMILES: *N1CCOCC1] | 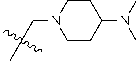 [SMILES: *CN1CCC(N(C)C)CC1] |
| 259 | —N— | —CH— | 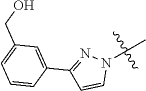 [SMILES: *n1ccc(-c2cccc(CO)c2)n1] | 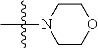 [SMILES: *N1CCOCC1] | 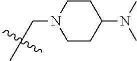 [SMILES: *CN1CCC(N(C)C)CC1] |
| 260 | —N— | —CH— | 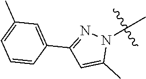 [SMILES: *n1nc(-c2cccc(C)c2)cc1C] | 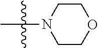 [SMILES: *N1CCOCC1] | 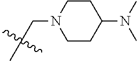 [SMILES: *CN1CCC(N(C)C)CC1] |
| 261 | —N— | —CH— | 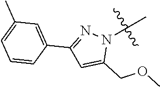 [SMILES: *n1nc(-c2cccc(C)c2)cc1COC] | 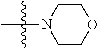 [SMILES: *N1CCOCC1] | 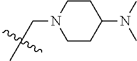 [SMILES: *CN1CCC(N(C)C)CC1] |
| 262 | —N— | —CH— | 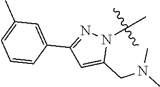 [SMILES: *n1nc(-c2cccc(C)c2)cc1CN(C)C] | 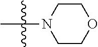 [SMILES: *N1CCOCC1] | 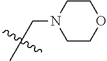 [SMILES: *CN1CCOCC1] |
| 263 | —N— | —CH— | 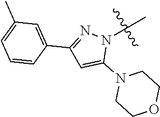 [SMILES: *n1nc(-c2cccc(C)c2)cc1N1CCOCC1] | 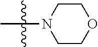 [SMILES: *N1CCOCC1] | 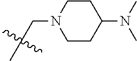 [SMILES: *CN1CCC(N(C)C)CC1] |
| 264 | —N— | —CH— | 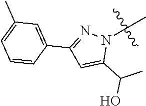 [SMILES: *n1nc(-c2cccc(C)c2)cc1C(C)O] | 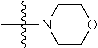 [SMILES: *N1CCOCC1] | 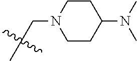 [SMILES: *CN1CCC(N(C)C)CC1] |
| 265 | —N— | —CH— | 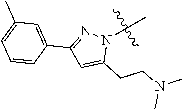 [SMILES: *n1nc(-c2cccc(C)c2)cc1CCN(C)C] | 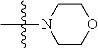 [SMILES: *N1CCOCC1] | 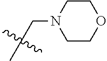 [SMILES: *CN1CCOCC1] |
| 266 | —N— | —CH— | 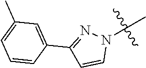 [SMILES: *n1ccc(-c2cccc(C)c2)n1] | 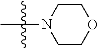 [SMILES: *N1CCOCC1] | 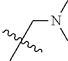 [SMILES: *CN(C)C] |
| 267 | —N— | —CH— | 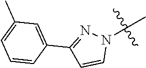 [SMILES: *n1ccc(-c2cccc(C)c2)n1] | 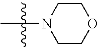 [SMILES: *N1CCOCC1] | 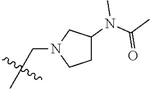 [SMILES: *CN1CCC(N(C)C(C)=O)C1] |
| 268 | —N— | —CH— | 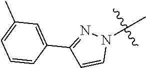 [SMILES: *n1ccc(-c2cccc(C)c2)n1] | 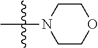 [SMILES: *N1CCOCC1] | 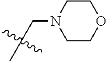 [SMILES: *CN1CCOCC1] |
| 269 | —N— | —CH— | 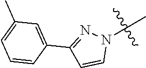 [SMILES: *n1ccc(-c2cccc(C)c2)n1] | 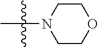 [SMILES: *N1CCOCC1] | 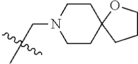 [SMILES: *CN1CCC2(CCCO2)CC1] |
| 270 | —N— | —CH— | 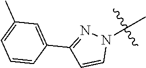 [SMILES: *n1ccc(-c2cccc(C)c2)n1] | 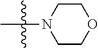 [SMILES: *N1CCOCC1] | 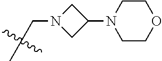 [SMILES: *CN1CC(N2CCOCC2)C1] |
| 271 | —N— | —CH— | 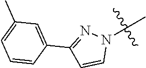 [SMILES: *n1ccc(-c2cccc(C)c2)n1] | 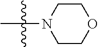 [SMILES: *N1CCOCC1] | 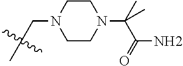 [SMILES: *CN1CCN(C(C)(C)C(N)=O)CC1] |
| 272 | —N— | —CH— | 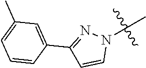 [SMILES: *n1ccc(-c2cccc(C)c2)n1] | 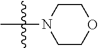 [SMILES: *N1CCOCC1] | 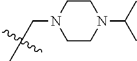 [SMILES: *CN1CCN(C(C)C)CC1] |
| 273 | —N— | —CH— | 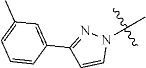 [SMILES: *n1ccc(-c2cccc(C)c2)n1] | 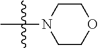 [SMILES: *N1CCOCC1] | 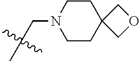 [SMILES: *CN1CCC2(CC1)COC2] |
| 274 | —N— | —CH— | 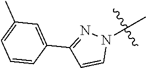 [SMILES: *n1ccc(-c2cccc(C)c2)n1] | 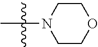 [SMILES: *N1CCOCC1] | 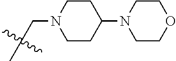 [SMILES: *CN1CCC(N2CCOCC2)CC1] |
| 275 | —N— | —CH— | 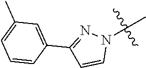 [SMILES: *n1ccc(-c2cccc(C)c2)n1] | 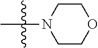 [SMILES: *N1CCOCC1] | 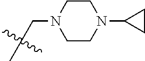 [SMILES: *CN1CCN(C2CC2)CC1] |
| 276 | —N— | —CH— | 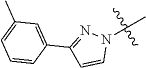 [SMILES: *n1ccc(-c2cccc(C)c2)n1] | 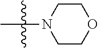 [SMILES: *N1CCOCC1] | 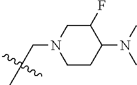 [SMILES: *CN1CCC(N(C)C)C(F)C1] |
| 277 | —N— | —CH— | 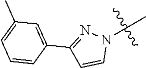 [SMILES: *n1ccc(-c2cccc(C)c2)n1] | 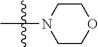 [SMILES: *N1CCOCC1] | 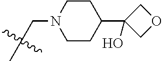 [SMILES: *CN1CCC(C2(O)COC2)CC1] |
| 278 | —N— | —CH— | 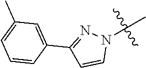 [SMILES: *n1ccc(-c2cccc(C)c2)n1] | 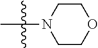 [SMILES: *N1CCOCC1] | 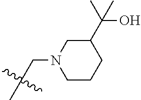 [SMILES: *CN1CCCC(C(C)(C)O)C1] |
| 279 | —N— | —CH— | 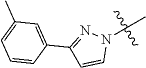 [SMILES: *n1ccc(-c2cccc(C)c2)n1] | 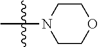 [SMILES: *N1CCOCC1] | 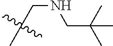 [SMILES: *CNCC(C)(C)C] |
| 280 | —N— | —CH— | 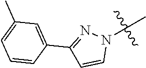 [SMILES: *n1ccc(-c2cccc(C)c2)n1] | 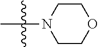 [SMILES: *N1CCOCC1] | 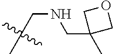 [SMILES: *CNCC1(C)COC1] |
| 281 | —N— | —CH— | 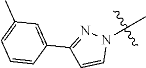 [SMILES: *n1ccc(-c2cccc(C)c2)n1] | 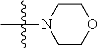 [SMILES: *N1CCOCC1] | 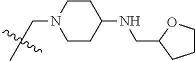 [SMILES: *CN1CCC(NCC2CCCO2)CC1] |
| 282 | —N— | —CH— | 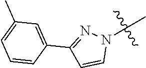 [SMILES: *n1ccc(-c2cccc(C)c2)n1] | 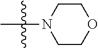 [SMILES: *N1CCOCC1] | 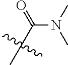 [SMILES: *C(=O)N(C)C] |
| 283 | —N— | —CH— | 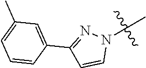 [SMILES: *n1ccc(-c2cccc(C)c2)n1] | 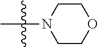 [SMILES: *N1CCOCC1] | 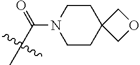 [SMILES: *C(=O)N1CCC2(CC1)COC2] |
| 284 | —N— | —CH— | 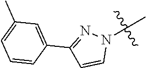 [SMILES: *n1ccc(-c2cccc(C)c2)n1] | 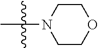 [SMILES: *N1CCOCC1] | 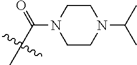 [SMILES: *C(=O)N1CCN(C(C)C)CC1] |
| 285 | —N— | —CH— | 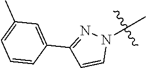 [SMILES: *n1ccc(-c2cccc(C)c2)n1] | 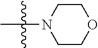 [SMILES: *N1CCOCC1] | 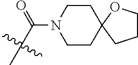 [SMILES: *C(=O)N1CCC2(CCCO2)CC1] |
| 286 | —N— | —CH— | 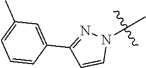 [SMILES: *n1ccc(-c2cccc(C)c2)n1] | 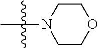 [SMILES: *N1CCOCC1] | 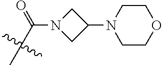 [SMILES: *C(=O)N1CC(N2CCOCC2)C1] |
| 287 | —N— | —CH— | 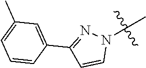 [SMILES: *n1ccc(-c2cccc(C)c2)n1] | 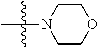 [SMILES: *N1CCOCC1] | 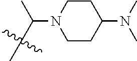 [SMILES: *C(C)N1CCC(N(C)C)CC1] |
| 288 | —N— | —CH— | 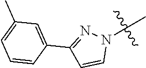 [SMILES: *n1ccc(-c2cccc(C)c2)n1] | 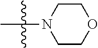 [SMILES: *N1CCOCC1] | 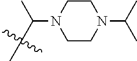 [SMILES: *C(C)N1CCN(C(C)C)CC1] |
| 289 | —N— | —CH— | 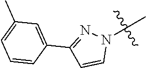 [SMILES: *n1ccc(-c2cccc(C)c2)n1] | 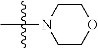 [SMILES: *N1CCOCC1] | 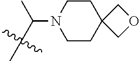 [SMILES: *C(C)N1CCC2(CC1)COC2] |
| 290 | —N— | —CH— | 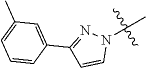 [SMILES: *n1ccc(-c2cccc(C)c2)n1] | 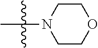 [SMILES: *N1CCOCC1] | 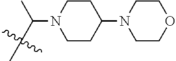 [SMILES: *C(C)N1CCC(N2CCOCC2)CC1] |
| 291 | —N— | —CH— | 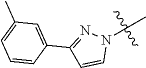 [SMILES: *n1ccc(-c2cccc(C)c2)n1] | 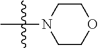 [SMILES: *N1CCOCC1] | 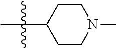 [SMILES: *C1CCN(C)CC1] |
| 292 | —N— | —CH— | 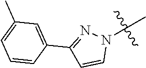 [SMILES: *n1ccc(-c2cccc(C)c2)n1] | 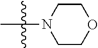 [SMILES: *N1CCOCC1] | 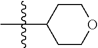 [SMILES: *C1CCOCC1] |
| 293 | —N— | —CH— | 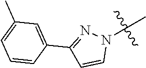 [SMILES: *n1ccc(-c2cccc(C)c2)n1] | 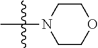 [SMILES: *N1CCOCC1] | 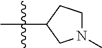 [SMILES: *C1CCN(C)C1] |
| 294 | —N— | —CH— | 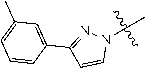 [SMILES: *n1ccc(-c2cccc(C)c2)n1] | 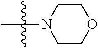 [SMILES: *N1CCOCC1] | 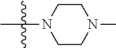 [SMILES: *N1CCN(C)CC1] |
| 295 | —N— | —CH— | 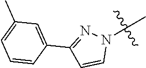 [SMILES: *n1ccc(-c2cccc(C)c2)n1] | 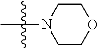 [SMILES: *N1CCOCC1] | 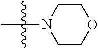 [SMILES: *N1CCOCC1] |
| 296 | —N— | —CH— | 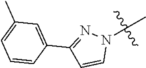 [SMILES: *n1ccc(-c2cccc(C)c2)n1] | 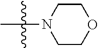 [SMILES: *N1CCOCC1] | 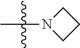 [SMILES: *N1CCC1] |
| 297 | —N— | —CH— | 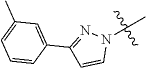 [SMILES: *n1ccc(-c2cccc(C)c2)n1] | 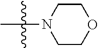 [SMILES: *N1CCOCC1] | 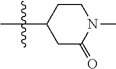 [SMILES: *C1CCN(C)C(=O)C1] |
| 298 | —N— | —CH— | 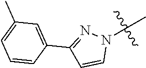 [SMILES: *n1ccc(-c2cccc(C)c2)n1] | 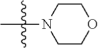 [SMILES: *N1CCOCC1] | 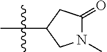 [SMILES: *C1CC(=O)N(C)C1] |
| 299 | —N— | —CH— | 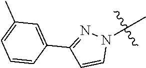 [SMILES: *n1ccc(-c2cccc(C)c2)n1] | 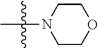 [SMILES: *N1CCOCC1] | 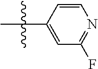 [SMILES: *c1ccnc(F)c1] |
| 300 | —N— | —CH— | 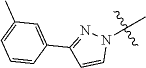 [SMILES: *n1ccc(-c2cccc(C)c2)n1] | 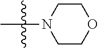 [SMILES: *N1CCOCC1] | 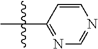 [SMILES: *c1ccncn1] |
| 301 | —N— | —CH— | 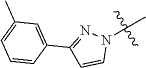 [SMILES: *n1ccc(-c2cccc(C)c2)n1] | 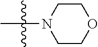 [SMILES: *N1CCOCC1] | 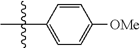 [SMILES: *c1ccc(OC)cc1] |
| 302 | —N— | —CH— | 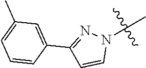 [SMILES: *n1ccc(-c2cccc(C)c2)n1] | 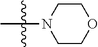 [SMILES: *N1CCOCC1] | 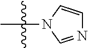 [SMILES: *n1ccnc1] |
| 303 | —N— | —CH— | 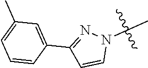 [SMILES: *n1ccc(-c2cccc(C)c2)n1] | 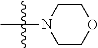 [SMILES: *N1CCOCC1] | 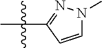 [SMILES: *c1ccn(C)n1] |
| 304 | —N— | —CH— | 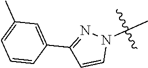 [SMILES: *n1ccc(-c2cccc(C)c2)n1] | 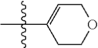 [SMILES: *C1=CCOCC1] | 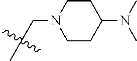 [SMILES: *CN1CCC(N(C)C)CC1] |
| 305 | —N— | —CH— | 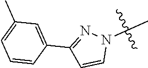 [SMILES: *n1ccc(-c2cccc(C)c2)n1] | 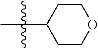 [SMILES: *C1CCOCC1] | 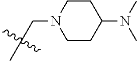 [SMILES: *CN1CCC(N(C)C)CC1] |
| 306 | —N— | —CH— | 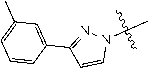 [SMILES: *n1ccc(-c2cccc(C)c2)n1] | 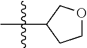 [SMILES: *C1CCOC1] | 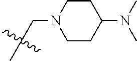 [SMILES: *CN1CCC(N(C)C)CC1] |
| 307 | —N— | —CH— | 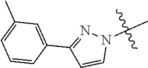 [SMILES: *n1ccc(-c2cccc(C)c2)n1] | 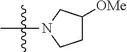 [SMILES: *N1CCC(OC)C1] | 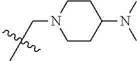 [SMILES: *CN1CCC(N(C)C)CC1] |
| 308 | —N— | —CH— | 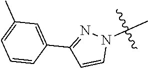 [SMILES: *n1ccc(-c2cccc(C)c2)n1] | 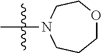 [SMILES: *N1CCCOCC1] | 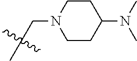 [SMILES: *CN1CCC(N(C)C)CC1] |
| 309 | —N— | —CH— | 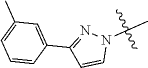 [SMILES: *n1ccc(-c2cccc(C)c2)n1] | 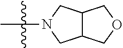 [SMILES: *N1CC2COCC2C1] | 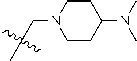 [SMILES: *CN1CCC(N(C)C)CC1] |
| 310 | —N— | —CH— | 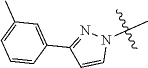 [SMILES: *n1ccc(-c2cccc(C)c2)n1] | 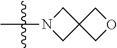 [SMILES: *N1CC2(COC2)C1] | 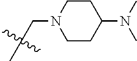 [SMILES: *CN1CCC(N(C)C)CC1] |
| 311 | —N— | —CH— | 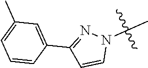 [SMILES: *n1ccc(-c2cccc(C)c2)n1] | 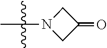 [SMILES: *N1CC(=O)C1] | 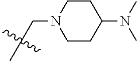 [SMILES: *CN1CCC(N(C)C)CC1] |
| 312 | —N— | —CH— | 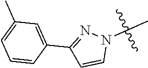 [SMILES: *n1ccc(-c2cccc(C)c2)n1] | 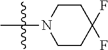 [SMILES: *N1CCC(F)(F)CC1] | 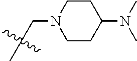 [SMILES: *CN1CCC(N(C)C)CC1] |
| 313 | —N— | —CH— | 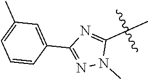 [SMILES: *c1nc(-c2cccc(C)c2)nn1C] | 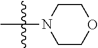 [SMILES: *N1CCOCC1] | 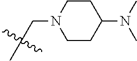 [SMILES: *CN1CCC(N(C)C)CC1] |
| 314 | —N— | —CH— | 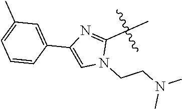 [SMILES: *c1nc(-c2cccc(C)c2)cn1CCN(C)C] | 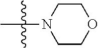 [SMILES: *N1CCOCC1] | 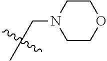 [SMILES: *CN1CCOCC1] |
| 315 | —N— | —CH— | 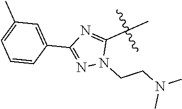 [SMILES: *c1nc(-c2cccc(C)c2)nn1CCN(C)C] | 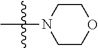 [SMILES: *N1CCOCC1] | 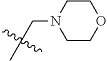 [SMILES: *CN1CCOCC1] |
| 316 | —N— | —CH— | 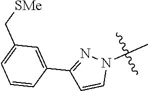 [SMILES: *n1ccc(-c2cccc(CSC)c2)n1] | 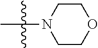 [SMILES: *N1CCOCC1] | 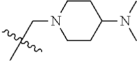 [SMILES: *CN1CCC(N(C)C)CC1] |
| 317 | —N— | —CH— | 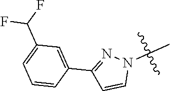 [SMILES: *n1ccc(-c2cccc(C(F)F)c2)n1] | 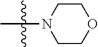 [SMILES: *N1CCOCC1] | 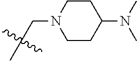 [SMILES: *CN1CCC(N(C)C)CC1] |
| 318 | —N— | —CH— | 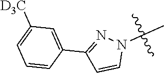 [SMILES: *n1ccc(-c2cccc(C([2H])([2H])[2H])c2)n1] | 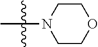 [SMILES: *N1CCOCC1] | 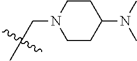 [SMILES: *CN1CCC(N(C)C)CC1] |
| 319 | —CH— | —CH— | 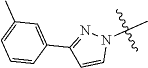 [SMILES: *n1ccc(-c2cccc(C)c2)n1] | 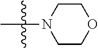 [SMILES: *N1CCOCC1] | 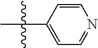 [SMILES: *c1ccncc1] |
| 320 | —CH— | —CH— | 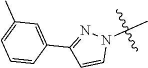 [SMILES: *n1ccc(-c2cccc(C)c2)n1] | 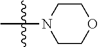 [SMILES: *N1CCOCC1] | 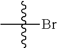 [SMILES: *Br] |
| 321 | —CH— | —CH— | 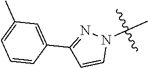 [SMILES: *n1ccc(-c2cccc(C)c2)n1] | 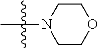 [SMILES: *N1CCOCC1] | 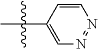 [SMILES: *c1ccnnc1] |
| 322 | —CH— | —CH— | 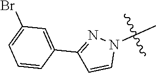 [SMILES: *n1ccc(-c2cccc(Br)c2)n1] | 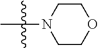 [SMILES: *N1CCOCC1] | 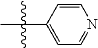 [SMILES: *c1ccncc1] |
| 323 | —CH— | —CH— | 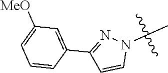 [SMILES: *n1ccc(-c2cccc(OC)c2)n1] | 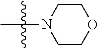 [SMILES: *N1CCOCC1] | 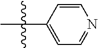 [SMILES: *c1ccncc1] |
| 324 | —CH— | —CH— | 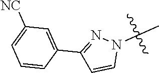 [SMILES: *n1ccc(-c2cccc(C#N)c2)n1] | 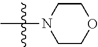 [SMILES: *N1CCOCC1] | 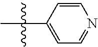 [SMILES: *c1ccncc1] |
| 325 | —CH— | —CH— | 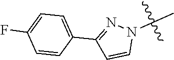 [SMILES: *n1ccc(-c2ccc(F)cc2)n1] | 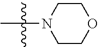 [SMILES: *N1CCOCC1] | 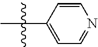 [SMILES: *c1ccncc1] |
| 326 | —CH— | —CH— | 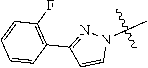 [SMILES: *n1ccc(-c2ccccc2F)n1] | 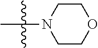 [SMILES: *N1CCOCC1] | 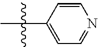 [SMILES: *c1ccncc1] |
| 327 | —CH— | —CH— | 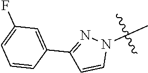 [SMILES: *n1ccc(-c2cccc(F)c2)n1] | 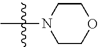 [SMILES: *N1CCOCC1] | 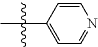 [SMILES: *c1ccncc1] |
| 328 | —CH— | —CH— | 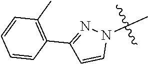 [SMILES: *n1ccc(-c2ccccc2C)n1] | 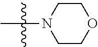 [SMILES: *N1CCOCC1] | 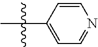 [SMILES: *c1ccncc1] |
| 329 | —CH— | —CH— | 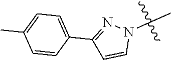 [SMILES: *n1ccc(-c2ccc(C)cc2)n1] | 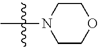 [SMILES: *N1CCOCC1] | 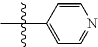 [SMILES: *c1ccncc1] |
| 330 | —CH— | —CH— | 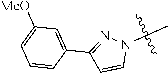 [SMILES: *n1ccc(-c2cccc(OC)c2)n1] | 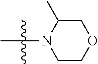 [SMILES: *N1CCOCC1C] | 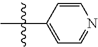 [SMILES: *c1ccncc1] |
| 331 | —CH— | —CH— | 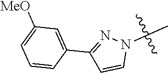 [SMILES: *n1ccc(-c2cccc(OC)c2)n1] | 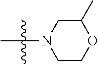 [SMILES: *N1CCOC(C)C1] | 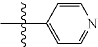 [SMILES: *c1ccncc1] |
| 332 | —CH— | —CH— | 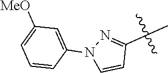 [SMILES: *c1ccn(-c2cccc(OC)c2)n1] | 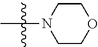 [SMILES: *N1CCOCC1] | 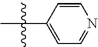 [SMILES: *c1ccncc1] |
| 333 | —CH— | —CH— | 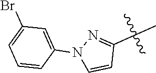 [SMILES: *c1ccn(-c2cccc(Br)c2)n1] | 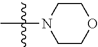 [SMILES: *N1CCOCC1] | 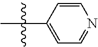 [SMILES: *c1ccncc1] |
| 334 | —CH— | —CH— | 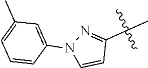 [SMILES: *c1ccn(-c2cccc(C)c2)n1] | 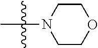 [SMILES: *N1CCOCC1] | 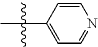 [SMILES: *c1ccncc1] |
| 335 | —CH— | —CH— | 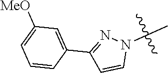 [SMILES: *n1ccc(-c2cccc(OC)c2)n1] | 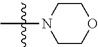 [SMILES: *N1CCOCC1] | 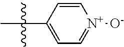 [SMILES: *c1cc[n+]([O-])cc1] |
| 336 | —CH— | —CH— | 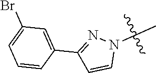 [SMILES: *n1ccc(-c2cccc(Br)c2)n1] | 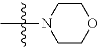 [SMILES: *N1CCOCC1] | 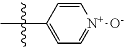 [SMILES: *c1cc[n+]([O-])cc1] |
| 337 | —CH— | —CH— | 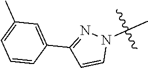 [SMILES: *n1ccc(-c2cccc(C)c2)n1] | 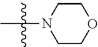 [SMILES: *N1CCOCC1] | 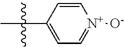 [SMILES: *c1cc[n+]([O-])cc1] |
| 338 | —CH— | —CH— | 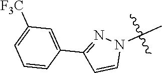 [SMILES: *n1ccc(-c2cccc(C(F)(F)F)c2)n1] | 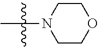 [SMILES: *N1CCOCC1] | 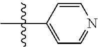 [SMILES: *c1ccncc1] |
| 339 | —CH— | —CH— | 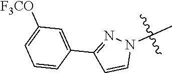 [SMILES: *n1ccc(-c2cccc(OC(F)(F)F)c2)n1] | 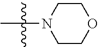 [SMILES: *N1CCOCC1] | 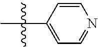 [SMILES: *c1ccncc1] |
| 340 | —CH— | —CH— | 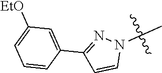 [SMILES: *n1ccc(-c2cccc(OCC)c2)n1] | 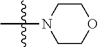 [SMILES: *N1CCOCC1] | 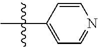 [SMILES: *c1ccncc1] |
| 341 | —CH— | —CH— | 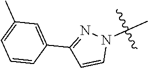 [SMILES: *n1ccc(-c2cccc(C)c2)n1] | 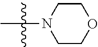 [SMILES: *N1CCOCC1] | 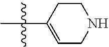 [SMILES: *C1=CCNCC1] |
| 342 | —CH— | —CH— | 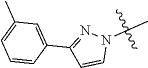 [SMILES: *n1ccc(-c2cccc(C)c2)n1] | 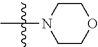 [SMILES: *N1CCOCC1] | 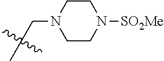 [SMILES: *CN1CCN(S(C)(=O)=O)CC1] |
| 343 | —CH— | —CH— | 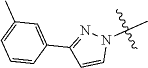 [SMILES: *n1ccc(-c2cccc(C)c2)n1] | 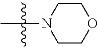 [SMILES: *N1CCOCC1] | 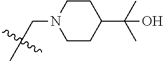 [SMILES: *CN1CCC(C(C)(C)O)CC1] |
| 344 | —CH— | —CH— | 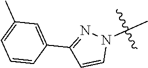 [SMILES: *n1ccc(-c2cccc(C)c2)n1] | 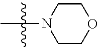 [SMILES: *N1CCOCC1] | 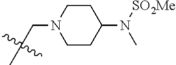 [SMILES: *CN1CCC(N(C)S(C)(=O)=O)CC1] |
| 345 | —CH— | —CH— | 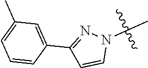 [SMILES: *n1ccc(-c2cccc(C)c2)n1] | 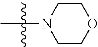 [SMILES: *N1CCOCC1] | 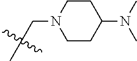 [SMILES: *CN1CCC(N(C)C)CC1] |
| 346 | —CH— | —CH— | 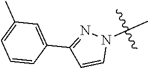 [SMILES: *n1ccc(-c2cccc(C)c2)n1] | 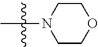 [SMILES: *N1CCOCC1] | 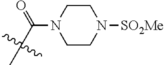 [SMILES: *C(=O)N1CCN(S(C)(=O)=O)CC1] |
| 347 | —CH— | —CH— | 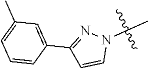 [SMILES: *n1ccc(-c2cccc(C)c2)n1] | 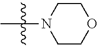 [SMILES: *N1CCOCC1] | 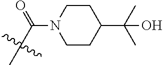 [SMILES: *C(=O)N1CCC(C(C)(C)O)CC1] |
| 348 | —CH— | —CH— | 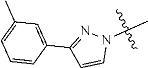 [SMILES: *n1ccc(-c2cccc(C)c2)n1] | 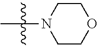 [SMILES: *N1CCOCC1] | 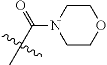 [SMILES: *C(=O)N1CCOCC1] |
| 349 | —CH— | —CH— | 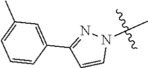 [SMILES: *n1ccc(-c2cccc(C)c2)n1] | 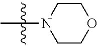 [SMILES: *N1CCOCC1] | 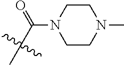 [SMILES: *C(=O)N1CCN(C)CC1] |
| 350 | —CH— | —CH— | 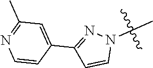 [SMILES: *n1ccc(-c2ccnc(C)c2)n1] | 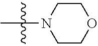 [SMILES: *N1CCOCC1] | 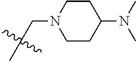 [SMILES: *CN1CCC(N(C)C)CC1] |
| 351 | —CH— | —CH— | 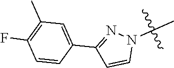 [SMILES: *n1ccc(-c2ccc(F)c(C)c2)n1] | 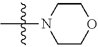 [SMILES: *N1CCOCC1] | 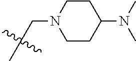 [SMILES: *CN1CCC(N(C)C)CC1] |
| 352 | —CH— | —CH— | 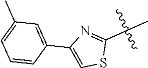 [SMILES: *c1nc(-c2cccc(C)c2)cs1] | 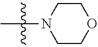 [SMILES: *N1CCOCC1] | 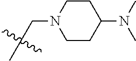 [SMILES: *CN1CCC(N(C)C)CC1] |
| 353 | —CH— | —CH— | 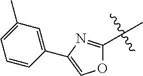 [SMILES: *c1nc(-c2cccc(C)c2)co1] | 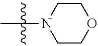 [SMILES: *N1CCOCC1] | 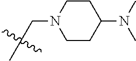 [SMILES: *CN1CCC(N(C)C)CC1] |
| 354 | —CH— | —CH— | 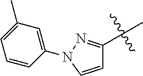 [SMILES: *c1ccn(-c2cccc(C)c2)n1] | 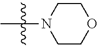 [SMILES: *N1CCOCC1] | 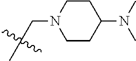 [SMILES: *CN1CCC(N(C)C)CC1] |
| 355 | —CH— | —CH— | 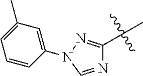 [SMILES: *c1ncn(-c2cccc(C)c2)n1] | 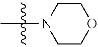 [SMILES: *N1CCOCC1] | 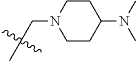 [SMILES: *CN1CCC(N(C)C)CC1] |
| 356 | —CH— | —CH— | 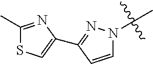 [SMILES: *n1ccc(-c2csc(C)n2)n1] | 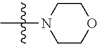 [SMILES: *N1CCOCC1] | 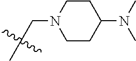 [SMILES: *CN1CCC(N(C)C)CC1] |
| 357 | —CH— | —CH— | 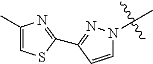 [SMILES: *n1ccc(-c2nc(C)cs2)n1] | 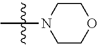 [SMILES: *N1CCOCC1] | 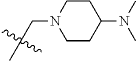 [SMILES: *CN1CCC(N(C)C)CC1] |
| 358 | —CH— | —CH— | 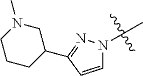 [SMILES: *n1ccc(C2CCCN(C)C2)n1] | 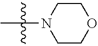 [SMILES: *N1CCOCC1] | 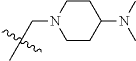 [SMILES: *CN1CCC(N(C)C)CC1] |
| 359 | —CH— | —CH— | 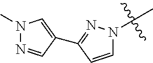 [SMILES: *n1ccc(-c2cnn(C)c2)n1] | 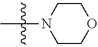 [SMILES: *N1CCOCC1] | 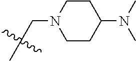 [SMILES: *CN1CCC(N(C)C)CC1] |
| 360 | —CH— | —CH— | 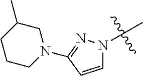 [SMILES: *n1ccc(N2CCCC(C)C2)n1] | 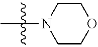 [SMILES: *N1CCOCC1] | 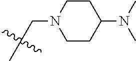 [SMILES: *CN1CCC(N(C)C)CC1] |
| 361 | —CH— | —CH— | 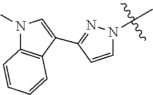 [SMILES: *n1ccc(-c2cn(C)c3ccccc23)n1] | 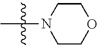 [SMILES: *N1CCOCC1] | 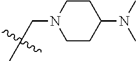 [SMILES: *CN1CCC(N(C)C)CC1] |
| 362 | —CH— | —CH— | 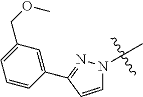 [SMILES: *n1ccc(-c2cccc(COC)c2)n1] | 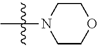 [SMILES: *N1CCOCC1] | 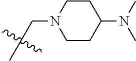 [SMILES: *CN1CCC(N(C)C)CC1] |
| 363 | —CH— | —CH— | 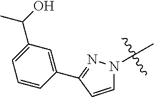 [SMILES: *n1ccc(-c2cccc(C(C)O)c2)n1] | 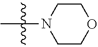 [SMILES: *N1CCOCC1] | 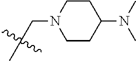 [SMILES: *CN1CCC(N(C)C)CC1] |
| 364 | —CH— | —CH— | 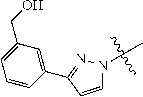 [SMILES: *n1ccc(-c2cccc(CO)c2)n1] | 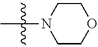 [SMILES: *N1CCOCC1] | 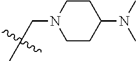 [SMILES: *CN1CCC(N(C)C)CC1] |
| 365 | —CH— | —CH— | 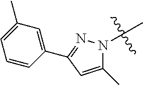 [SMILES: *n1nc(-c2cccc(C)c2)cc1C] | 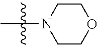 [SMILES: *N1CCOCC1] | 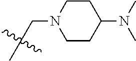 [SMILES: *CN1CCC(N(C)C)CC1] |
| 366 | —CH— | —CH— | 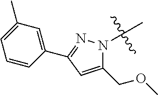 [SMILES: *n1nc(-c2cccc(C)c2)cc1COC] | 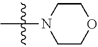 [SMILES: *N1CCOCC1] | 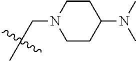 [SMILES: *CN1CCC(N(C)C)CC1] |
| 367 | —CH— | —CH— | 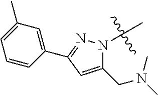 [SMILES: *n1nc(-c2cccc(C)c2)cc1CN(C)C] | 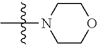 [SMILES: *N1CCOCC1] | 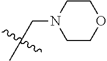 [SMILES: *CN1CCOCC1] |
| 368 | —CH— | —CH— | 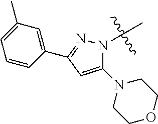 [SMILES: *n1nc(-c2cccc(C)c2)cc1N1CCOCC1] | 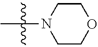 [SMILES: *N1CCOCC1] | 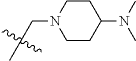 [SMILES: *CN1CCC(N(C)C)CC1] |
| 369 | —CH— | —CH— | 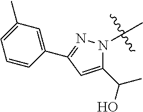 [SMILES: *n1nc(-c2cccc(C)c2)cc1C(C)O] | 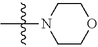 [SMILES: *N1CCOCC1] | 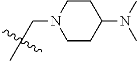 [SMILES: *CN1CCC(N(C)C)CC1] |
| 370 | —CH— | —CH— | 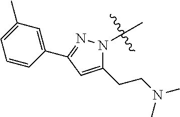 [SMILES: *n1nc(-c2cccc(C)c2)cc1CCN(C)C] | 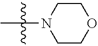 [SMILES: *N1CCOCC1] | 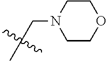 [SMILES: *CN1CCOCC1] |
| 371 | —CH— | —CH— | 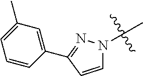 [SMILES: *n1ccc(-c2cccc(C)c2)n1] | 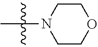 [SMILES: *N1CCOCC1] | 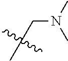 [SMILES: *CN(C)C] |
| 372 | —CH— | —CH— | 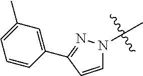 [SMILES: *n1ccc(-c2cccc(C)c2)n1] | 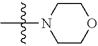 [SMILES: *N1CCOCC1] | 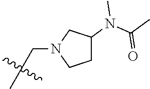 [SMILES: *CN1CCC(N(C)C(C)=O)C1] |
| 373 | —CH— | —CH— | 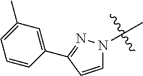 [SMILES: *n1ccc(-c2cccc(C)c2)n1] | 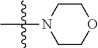 [SMILES: *N1CCOCC1] | 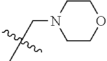 [SMILES: *CN1CCOCC1] |
| 374 | —CH— | —CH— | 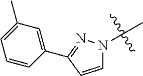 [SMILES: *n1ccc(-c2cccc(C)c2)n1] | 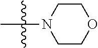 [SMILES: *N1CCOCC1] | 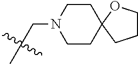 [SMILES: *CN1CCC2(CCCO2)CC1] |
| 375 | —CH— | —CH— | 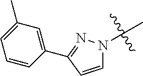 [SMILES: *n1ccc(-c2cccc(C)c2)n1] | 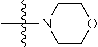 [SMILES: *N1CCOCC1] | 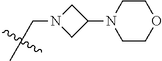 [SMILES: *CN1CC(N2CCOCC2)C1] |
| 376 | —CH— | —CH— | 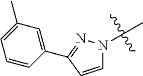 [SMILES: *n1ccc(-c2cccc(C)c2)n1] | 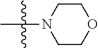 [SMILES: *N1CCOCC1] | 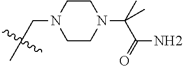 [SMILES: *CN1CCN(C(C)(C)C(N)=O)CC1] |
| 377 | —CH— | —CH— | 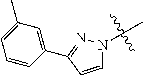 [SMILES: *n1ccc(-c2cccc(C)c2)n1] | 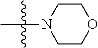 [SMILES: *N1CCOCC1] | 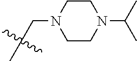 [SMILES: *CN1CCN(C(C)C)CC1] |
| 378 | —CH— | —CH— | 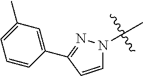 [SMILES: *n1ccc(-c2cccc(C)c2)n1] | 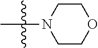 [SMILES: *N1CCOCC1] | 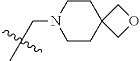 [SMILES: *CN1CCC2(CC1)COC2] |
| 379 | —CH— | —CH— | 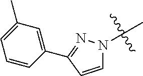 [SMILES: *n1ccc(-c2cccc(C)c2)n1] | 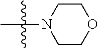 [SMILES: *N1CCOCC1] | 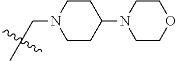 [SMILES: *CN1CCC(N2CCOCC2)CC1] |
| 380 | —CH— | —CH— | 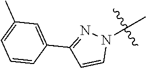 [SMILES: *n1ccc(-c2cccc(C)c2)n1] | 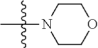 [SMILES: *N1CCOCC1] | 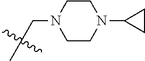 [SMILES: *CN1CCN(C2CC2)CC1] |
| 381 | —CH— | —CH— | 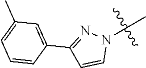 [SMILES: *n1ccc(-c2cccc(C)c2)n1] | 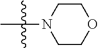 [SMILES: *N1CCOCC1] | 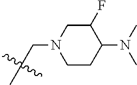 [SMILES: *CN1CCC(N(C)C)C(F)C1] |
| 382 | —CH— | —CH— | 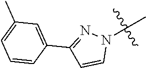 [SMILES: *n1ccc(-c2cccc(C)c2)n1] | 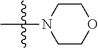 [SMILES: *N1CCOCC1] | 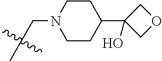 [SMILES: *CN1CCC(C2(O)COC2)CC1] |
| 383 | —CH— | —CH— | 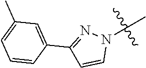 [SMILES: *n1ccc(-c2cccc(C)c2)n1] | 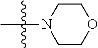 [SMILES: *N1CCOCC1] | 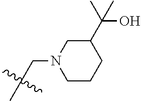 [SMILES: *CN1CCCC(C(C)(C)O)C1] |
| 384 | —CH— | —CH— | 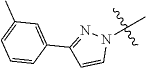 [SMILES: *n1ccc(-c2cccc(C)c2)n1] | 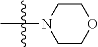 [SMILES: *N1CCOCC1] | 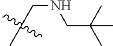 [SMILES: *CNCC(C)(C)C] |
| 385 | —CH— | —CH— | 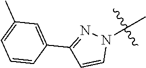 [SMILES: *n1ccc(-c2cccc(C)c2)n1] | 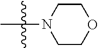 [SMILES: *N1CCOCC1] | 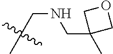 [SMILES: *CNCC1(C)COC1] |
| 386 | —CH— | —CH— | 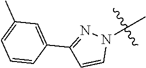 [SMILES: *n1ccc(-c2cccc(C)c2)n1] | 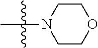 [SMILES: *N1CCOCC1] | 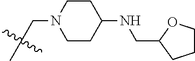 [SMILES: *CN1CCC(NCC2CCCO2)CC1] |
| 387 | —CH— | —CH— | 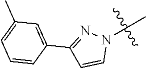 [SMILES: *n1ccc(-c2cccc(C)c2)n1] | 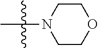 [SMILES: *N1CCOCC1] | 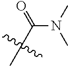 [SMILES: *C(=O)N(C)C] |
| 388 | —CH— | —CH— | 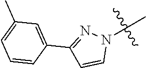 [SMILES: *n1ccc(-c2cccc(C)c2)n1] | 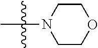 [SMILES: *N1CCOCC1] | 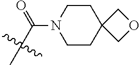 [SMILES: *C(=O)N1CCC2(CC1)COC2] |
| 389 | —CH— | —CH— | 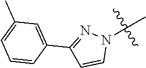 [SMILES: *n1ccc(-c2cccc(C)c2)n1] | 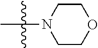 [SMILES: *N1CCOCC1] | 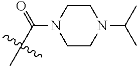 [SMILES: *C(=O)N1CCN(C(C)C)CC1] |
| 390 | —CH— | —CH— | 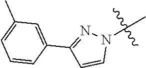 [SMILES: *n1ccc(-c2cccc(C)c2)n1] | 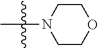 [SMILES: *N1CCOCC1] | 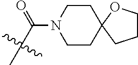 [SMILES: *C(=O)N1CCC2(CCCO2)CC1] |
| 391 | —CH— | —CH— | 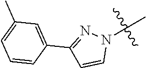 [SMILES: *n1ccc(-c2cccc(C)c2)n1] | 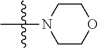 [SMILES: *N1CCOCC1] | 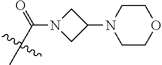 [SMILES: *C(=O)N1CC(N2CCOCC2)C1] |
| 392 | —CH— | —CH— | 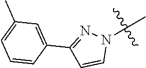 [SMILES: *n1ccc(-c2cccc(C)c2)n1] | 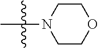 [SMILES: *N1CCOCC1] | 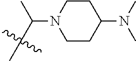 [SMILES: *C(C)N1CCC(N(C)C)CC1] |
| 393 | —CH— | —CH— | 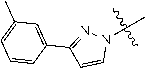 [SMILES: *n1ccc(-c2cccc(C)c2)n1] | 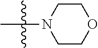 [SMILES: *N1CCOCC1] | 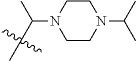 [SMILES: *C(C)N1CCN(C(C)C)CC1] |
| 394 | —CH— | —CH— | 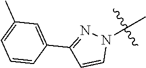 [SMILES: *n1ccc(-c2cccc(C)c2)n1] | 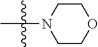 [SMILES: *N1CCOCC1] | 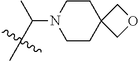 [SMILES: *C(C)N1CCC2(CC1)COC2] |
| 395 | —CH— | —CH— | 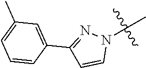 [SMILES: *n1ccc(-c2cccc(C)c2)n1] | 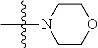 [SMILES: *N1CCOCC1] | 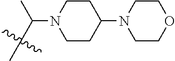 [SMILES: *C(C)N1CCC(N2CCOCC2)CC1] |
| 396 | —CH— | —CH— | 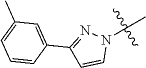 [SMILES: *n1ccc(-c2cccc(C)c2)n1] | 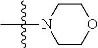 [SMILES: *N1CCOCC1] | 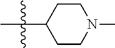 [SMILES: *C1CCN(C)CC1] |
| 397 | —CH— | —CH— | 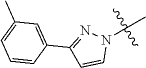 [SMILES: *n1ccc(-c2cccc(C)c2)n1] | 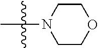 [SMILES: *N1CCOCC1] | 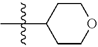 [SMILES: *C1CCOCC1] |
| 398 | —CH— | —CH— | 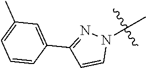 [SMILES: *n1ccc(-c2cccc(C)c2)n1] | 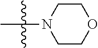 [SMILES: *N1CCOCC1] | 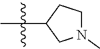 [SMILES: *C1CCN(C)C1] |
| 399 | —CH— | —CH— | 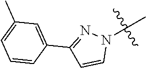 [SMILES: *n1ccc(-c2cccc(C)c2)n1] | 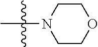 [SMILES: *N1CCOCC1] | 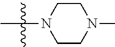 [SMILES: *N1CCN(C)CC1] |
| 400 | —CH— | —CH— | 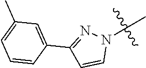 [SMILES: *n1ccc(-c2cccc(C)c2)n1] | 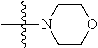 [SMILES: *N1CCOCC1] | 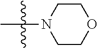 [SMILES: *N1CCOCC1] |
| 401 | —CH— | —CH— | 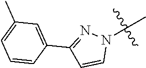 [SMILES: *n1ccc(-c2cccc(C)c2)n1] | 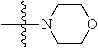 [SMILES: *N1CCOCC1] | 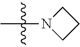 [SMILES: *N1CCC1] |
| 402 | —CH— | —CH— | 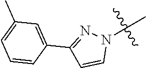 [SMILES: *n1ccc(-c2cccc(C)c2)n1] | 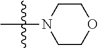 [SMILES: *N1CCOCC1] | 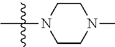 [SMILES: *N1CCN(C)CC1] |
| 403 | —CH— | —CH— | 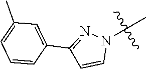 [SMILES: *n1ccc(-c2cccc(C)c2)n1] | 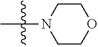 [SMILES: *N1CCOCC1] | 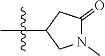 [SMILES: *C1CC(=O)N(C)C1] |
| 404 | —CH— | —CH— | 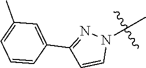 [SMILES: *n1ccc(-c2cccc(C)c2)n1] | 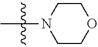 [SMILES: *N1CCOCC1] | 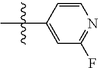 [SMILES: *c1ccnc(F)c1] |
| 405 | —CH— | —CH— | 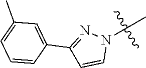 [SMILES: *n1ccc(-c2cccc(C)c2)n1] | 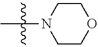 [SMILES: *N1CCOCC1] | 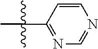 [SMILES: *c1ccncn1] |
| 406 | —CH— | —CH— | 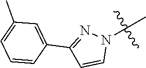 [SMILES: *n1ccc(-c2cccc(C)c2)n1] | 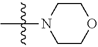 [SMILES: *N1CCOCC1] | 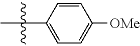 [SMILES: *c1ccc(OC)cc1] |
| 407 | —CH— | —CH— | 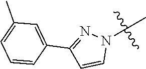 [SMILES: *n1ccc(-c2cccc(C)c2)n1] | 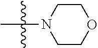 [SMILES: *N1CCOCC1] | 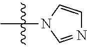 [SMILES: *n1ccnc1] |
| 408 | —CH— | —CH— | 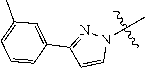 [SMILES: *n1ccc(-c2cccc(C)c2)n1] | 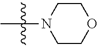 [SMILES: *N1CCOCC1] | 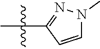 [SMILES: *c1ccn(C)n1] |
| 409 | —CH— | —CH— | 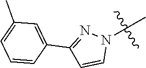 [SMILES: *n1ccc(-c2cccc(C)c2)n1] | 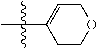 [SMILES: *C1=CCOCC1] | 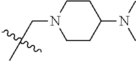 [SMILES: *CN1CCC(N(C)C)CC1] |
| 410 | —CH— | —CH— | 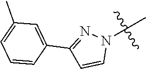 [SMILES: *n1ccc(-c2cccc(C)c2)n1] | 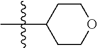 [SMILES: *C1CCOCC1] | 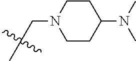 [SMILES: *CN1CCC(N(C)C)CC1] |
| 411 | —CH— | —CH— | 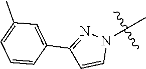 [SMILES: *n1ccc(-c2cccc(C)c2)n1] | 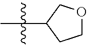 [SMILES: *C1CCOC1] | 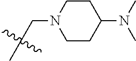 [SMILES: *CN1CCC(N(C)C)CC1] |
| 412 | —CH— | —CH— | 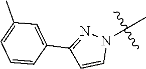 [SMILES: *n1ccc(-c2cccc(C)c2)n1] | 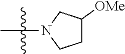 [SMILES: *N1CCC(OC)C1] | 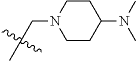 [SMILES: *CN1CCC(N(C)C)CC1] |
| 413 | —CH— | —CH— | 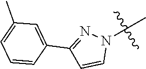 [SMILES: *n1ccc(-c2cccc(C)c2)n1] | 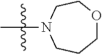 [SMILES: *N1CCCOCC1] | 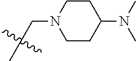 [SMILES: *CN1CCC(N(C)C)CC1] |
| 414 | —CH— | —CH— | 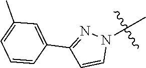 [SMILES: *n1ccc(-c2cccc(C)c2)n1] | 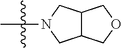 [SMILES: *N1CC2COCC2C1] | 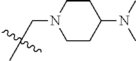 [SMILES: *CN1CCC(N(C)C)CC1] |
| 415 | —CH— | —CH— | 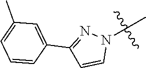 [SMILES: *n1ccc(-c2cccc(C)c2)n1] | 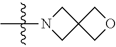 [SMILES: *N1CC2(COC2)C1] | 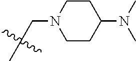 [SMILES: *CN1CCC(N(C)C)CC1] |
| 416 | —CH— | —CH— | 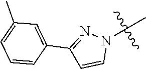 [SMILES: *n1ccc(-c2cccc(C)c2)n1] | 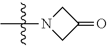 [SMILES: *N1CC(=O)C1] | 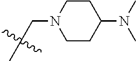 [SMILES: *CN1CCC(N(C)C)CC1] |
| 417 | —CH— | —CH— | 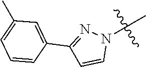 [SMILES: *n1ccc(-c2cccc(C)c2)n1] | 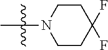 [SMILES: *N1CCC(F)(F)CC1] | 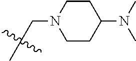 [SMILES: *CN1CCC(N(C)C)CC1] |
| 418 | —CH— | —CH— | 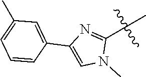 [SMILES: *c1nc(-c2cccc(C)c2)cn1C] | 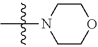 [SMILES: *N1CCOCC1] | 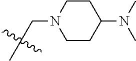 [SMILES: *CN1CCC(N(C)C)CC1] |
| 419 | —CH— | —CH— | 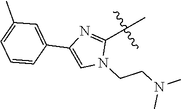 [SMILES: *c1nc(-c2cccc(C)c2)cn1CCN(C)C] | 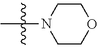 [SMILES: *N1CCOCC1] | 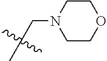 [SMILES: *CN1CCOCC1] |
| 420 | —CH— | —CH— | 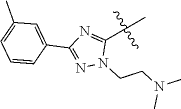 [SMILES: *c1nc(-c2cccc(C)c2)nn1CCN(C)C] | 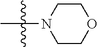 [SMILES: *N1CCOCC1] | 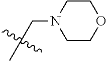 [SMILES: *CN1CCOCC1] |
| 421 | —CH— | —CH— | 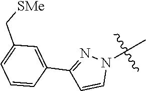 [SMILES: *n1ccc(-c2cccc(CSC)c2)n1] | 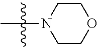 [SMILES: *N1CCOCC1] | 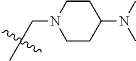 [SMILES: *CN1CCC(N(C)C)CC1] |
| 422 | —CH— | —CH— | 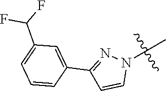 [SMILES: *n1ccc(-c2cccc(C(F)F)c2)n1] | 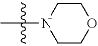 [SMILES: *N1CCOCC1] | 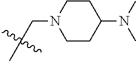 [SMILES: *CN1CCC(N(C)C)CC1] |
| 423 | —CH— | —CH— | 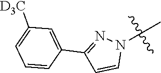 [SMILES: *n1ccc(-c2cccc(C([2H])([2H])[2H])c2)n1] | 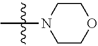 [SMILES: *N1CCOCC1] | 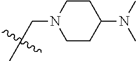 [SMILES: *CN1CCC(N(C)C)CC1] |

### Example 424: In Vitro Inhibitory Activity

Compounds were tested for PIKFYVE inhibitory activity using the ADP-GLO™ Kinase Assay (Carna Biosciences, Inc.) or the KINOMEScan™ Kinase Assay (Eurofins).

### ADP-GLO™ Kinase Assay

Briefly, 4× compound solution and 4×ATP solution are prepared with assay buffer (50 mM MOPS, 1 mM DTT, pH7.2). 4× Substrate solution and 4× kinase/Metal solution are prepared with MOPS based buffer containing individual kinase specific additives. Then, L of 4× compound solution, 5 μL of 4× Substrate solution, 5 μL of 4×ATP solution, and 5 μL of 4× kinase/Metal solution are mixed and incubated in a well of polystyrene 384 well black microplate for 1 hour at room temperature. Next, 20 μL of ADP-Glo™ Reagent (Promega) is added to the well, and incubated for over 40 minutes. 40 μL of Kinase Detection Reagent (Promega) is added to the well, and incubated for over 40 minutes. The kinase reaction is evaluated by the endpoint luminescence of the well.

For measurement of PIKFYVE activity, the substrate is PI3P at 10,000 nM concentration, using AG-182 as a positive control. The metal used is magnesium at 5 mM concentration.

### KINOMEscan™ Kinase Assay

Kinase-tagged T7 phage strains are prepared in an E. coli host derived from the BL21 strain. E. coli are grown to log-phase and infected with T7 phage, and incubated with shaking at 32° C. until lysis. The lysates are centrifuged and filtered to remove cell debris. Alternatively, some kinases are produced in HEK-293 cells and subsequently tagged with DNA for qPCR detection.

Streptavidin-coated magnetic beads are treated with biotinylated small molecule ligands for 30 minutes at room temperature to generate affinity resins for kinase assays. The liganded beads are blocked with excess biotin and washed with blocking buffer (Sea Block, 1% BSA, 0.05% Tween-20, 1 mM DTT) to remove unbound ligand and to reduce non-specific binding. Binding reactions are assembled by combining kinases, liganded affinity beads, and test compounds in 1× binding buffer (20% Sea Block, 0.17×PBS, 0.05% Tween-20, 6 mM DTT). Test compounds are prepared as 111× stocks in DMSO.

Kd values are determined using an 11-point, 3-fold compound dilution series with three DMSO control points. Compounds are distributed by non-contact acoustic transfer and are then directly diluted into the assay for a final concentration of DMSO of 0.9%, with a final volume in each well of 0.02 mL. Assay plates are incubated at room temperature with shaking for one hour, and the affinity beads are then washed with wash buffer (1% PBS, 0.05% Tween-20). The beads are then resuspended in elution buffer (lx PBS, 0.05% Tween-20, 0.05 μM non-biotinylated affinity ligand) and incubated with shaking for 30 minutes. The concentration of kinase in the eluates is measured by qPCR. Binding constants (Kd) are calculated with a standard dose-response curve using the Hill equation, fitted using non-linear least squares fit with the Levenberg-Marquedt algorithm.

The results are presented in the following table:

PIKFYVE Kinase Assays

| Compound | Activity (IC50)* | % Inhib. (50 nM)** | Kd (nM)** |
|---|---|---|---|
| Ex. 1 | +++ | | +++ |
| Ex. 2 | +++ | ++ | |
| Ex. 3 | +++ | +++ | |
| Ex. 4 | +++ | ++ | |
| Ex. 5 | + | + | |
| Ex. 6 | +++ | | +++ |
| Ex. 7 | ++ | + | |
| Ex. 8 | +++ | ++ | |
| Ex. 9 | +++ | ++ | |
| Ex. 10 | + | + | |
| Ex. 11 | | | + |
| Ex. 12 | | +++ | |
| Ex. 13 | | +++ | |
| Ex. 14 | | | + |
| Ex. 15 | | | ++ |
| Ex. 17 | | | +++ |
| Ex. 18 | | | +++ |
| Ex. 19 | | | +++ |
| Ex. 20 | | | +++ |
| Ex. 21 | | ++ | |
| Ex. 22 | | + | |
| Ex. 23 | | + | |
| Ex. 24 | | | ++ |
| Ex. 25 | | | +++ |
| Ex. 26 | | +++ | |
| Ex. 27 | | +++ | |
| Ex. 28 | | | ++ |
| Ex. 29 | | +++ | |

Key:

| | | | |
|---|---|---|---|
| +++ | <50 nM | >80%-100% | <1 nM |
| ++ | 50-1000 nM | >50%-80% | 1 nM-<5 nM |
| + | >1000 nM | <50% | 5-10 nM |

*ADP-GLO Kinase Assay;
**KINOMEScan Kinase Assay

These results demonstrate that the Compounds of the Invention are highly potent inhibitors of PIKFYVE. To determine whether the compounds are selective, the assay is repeated using appropriate substrates for different test enzymes:

| Enzyme | Substrate | Positive Control |
|---|---|---|
| PIK3CA | PI(4,5)P2 | PI-103 |
| PIP4K2A | PI5P | AG-183 |
| PIP4K2B | PI5P | AG-183 |
| PIP4K1A | PI4P | AG-183 |
| PIP4K1B | PI4P | AG-183 |
| PIP4K1C | PI4P | AG-183 |
| PIP5KL1 | PI4P | AG-183 |

The compounds of Examples 1, 3 and 6 are tested against these enzymes:

Comparative Kinase Assays

| Compound | Fold Selectivity for PIKFYVE |
|---|---|
| Ex. 6 | >200-fold |
| Ex. 1 | >100-fold |
| Ex. 3 | >50-fold |

### Example 425: Anti-Viral Activity

The compounds of Examples 1, 3, 6 and 19 were evaluated for their inhibitory activity against SARS-CoV-2 in the immunofluorescence assay (IFA) and/or against SARS-CoV-2 spike in the cell based pseudovirus reporter assay, and against Influenza A in an IFA assay.

SARS-CoV-2 IFA: In 384-well plates, Vero cells re seeded at an appropriate density and cultured at 37° C. and 5% CO2. Serially diluted (10 concentrations, in duplicates) test/reference compounds are added. Then the cells are infected with virus. The resulting cultures are incubated for an additional 1 day. The cells are then fixed and analyzed by immunofluorescence. EC50 and CC50 values are calculated with GraphPad Prism software.

SARS-CoV-2 Spike pseudovirus reporter assay: In 96-well plates, BHK21/hACE2 cells are seeded at an appropriate density and cultured at 37° C. and 5% CO2 overnight. Serially diluted (8 concentrations, in duplicates) test compounds are pre-incubated with cells at 37° C. for 1 hour. Then pseudoviruses is added into the cell cultures. The resulting cultures are kept at the same conditions for an additional 3 days. Britelite plus is used to measure the luciferase activity of pseudovirus for determination of activity of test compounds. EC50 and CC50 values are calculated with GraphPad Prism software.

Influenza A IFA: Test compounds are solubilized in DMSO to prepare 20 mg/mL stock solutions. Compounds are then serially diluted using eight half-log dilutions in test media so that the starting (high) test concentration is 100 μg/mL. Each dilution is added to 5 wells of a 96-well plate with 80-100% confluent MDCK cells. Three wells of each dilution are infected with virus (strain influenza A strain California/07/09 (H1N1) pdm09), and two wells remain uninfected as toxicity controls. As virus controls, six wells are infected but untreated, and as cell controls, six wells are uninfected and untreated. Each virus is prepared to achieve an MOI (multiplicity of infection) of 0.001. A positive control compound is tested in parallel. Plates are incubated at 37° C. under 5% CO₂ atmosphere. On day 3 post-infection (once untreated virus control cells reached maximum cytopathic effect, CPE), plates are stained with neutral red dye for 2 hours. Supernatant dye is removed and the wells are rinsed with PBS. The incorporated dye is extracted using 50:50 Sorensen citrate buffer/ethanol for at least 30 minutes, and the optical density is read at 450 nM on a spectrophotometer. Optical densities are converted to percent of cell controls and normalized to virus control, then the concentration of test compound required to inhibit CPE by 50% (EC₅₀) is calculated by regression analysis. The concentration of compound that causes 50% cell death in the absence of virus is also calculated (CC₅₀). The selectivity index (SI) is calculated as CC₅₀ divided by EC₅₀. Alternatively, the procedure above is followed, with the following minor modifications: Test compound is solubilized to form a 2 mg/mL DMSO stock solution; serial dilutions are prepared using a starting (high) test concentration of 10 μg/mL; maximum CPE is reached on day 5 post-infection.

The results are summarized in the tables below, with comparison to the reference compounds Apilimod, and chloroquine phosphate:

SARS-CoV2 Pseudovirus Reporter Assay

| | | |
|---|---|---|
| Test Material | EC50 | CC50 |

| | | | | |
|---|---|---|---|---|
| Ex. 1 | <10 | nM | 5-10 | μM |
| Ex. 3 | <10 | nM | >10 | μM |
| Ex. 6 | <10 | nM | 5-10 | μM |
| Apilimod | <10 | nM | 8 | μM |
| Chloroquine | 6 | μM | 35 | μM |

SARS-CoV2 IFA Assay

| Test Material | EC50 | CC50 |
|---|---|---|
| Ex. 1 | ++ | >20 μM |
| Ex. 3 | + | >20 μM |
| Ex. 19 | ++ | >200 μM |

Key

| | |
|---|---|
| + | >1 to 5 μM |
| ++ | 0.1 to 1 μM |
| +++ | <0.1 μM |

Influenza A IFA Assay

| Test Material | EC50 | CC50 |
|---|---|---|
| Ex. 1 | ++ | >20 μM |
| Ex. 3 | + | >20 μM |
| Ex. 19 | +++ | >200 μM |

Key

| | |
|---|---|
| + | >5 to 10 μM |
| ++ | >1 to 5 μM |
| +++ | 0.1 to 1 μM |
| ++++ | <0.1 μM |

The results demonstrate that the compounds of the invention have strong antiviral activity against the SARS-CoV-2 virus.

### Example 426: Anti-Proliferative Activity

Selected compounds are evaluated for their anti-proliferative activity against human cutaneous T-cell lymphoma and multiple myeloma cell lines.

HuT-78 (T cell lymphoma) cells or JJN3 (multiple myeloma) cells are cultured in RPMI-1640 with 10% FBS and 1% penicillin/streptomycin. 45 μL suspensions of cells are transferred to the wells of 384-well plate for a density of 1,000 cells per well. The plates are incubated overnight at 37° C. under 5% CO₂ atmosphere. Test compounds are dissolved in 100% DMSO at a 2 mM concentration, then diluted 20× in assay medium for to provide a 5% DMSO concentration and 100 μM compound concentration. 5 μL of this compound solution is added to the 45 μL cell suspension in the assay plate for a top plate concentration of 10 μM and 0.5% DMSO concentration. For positive controls, 10 μM APY0201 is added instead of the compound solution. After spinning at 1000 rpm for 1 minutes, the cell plate is placed in the incubator overnight at 37° C. under 5% CO₂ atmosphere. After incubation for six days, the cell plate is equilibrated to room temperature for 20 minutes. 25 μL of CellTiter-Glo (CTG) reagent (Promega Cat No. G7572) is added to each well, and the plate is shaken for 15 minutes at 300 rpm. Luminescence is read on an Envision plate reader.

The results are summarized in the table below:

Anti-Proliferative Assay

| Test Material | Hut78, IC50 | JJN3, IC50 |
|---|---|---|
| Ex. 1 | ++ | +++ |
| Ex. 6 | ++ | +++ |
| Ex. 19 | ++ | +++ |
| Ex. 20 | ++ | +++ |

Key

| | |
|---|---|
| + | >1 to 5 μM |
| ++ | 0.1 to 1 μM |
| +++ | <0.1 μM |

The results demonstrate that the compounds of the invention have strong anti-proliferative activity against cancer cells.

1. A compound of Formula I: 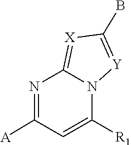
in free or pharmaceutically acceptable salt form, wherein
(i) X is —CH— or —N— (e.g., —CH—);
(ii) Y is —CH— or —N— (e.g., —N—);
(iii) A is an optionally substituted heteroaryl (e.g., 5-membered heteroaryl) or optionally substituted heterocycloalkyl (e.g., 3- to 6-membered heterocycloalkyl);
(iv) B is halo, an optionally substituted aryl, optionally substituted heteroaryl, optionally substituted C₃₋₆cycloalkyl, optionally substituted 3- to 6-membered heterocycloalkyl, optionally substituted 3- to 6-membered heterocycloalkenyl, optionally substituted C₁₋₆alkyl, optionally substituted C₂₋₆alkenyl (e.g., vinyl), —N(Rₐ)—R₂, —O—R₂, —(CO)—R₂, —(CO)—O—R₂, —(CO)—N(Rₐ)—R₂, —O—(CO)—R₂, —N(Rₐ)—(CO)—R₂, —(CO)—N(Rₐ)—(CO)—R₂, N(Rₐ)—(CO)—N(Rₐ)—R₂, optionally substituted —(C₁₋₆alkyl)-(3- to 6-membered heterocycloalkyl), optionally substituted —(C₁₋₆alkyl)-(C₃₋₆cycloalkyl), optionally substituted —(C₁₋₆alkyl)-N(Rₐ)—R₂, optionally substituted —(C₁₋₆alkyl)-O—R₂, optionally substituted —(C₁₋₆alkyl)-(CO)—N(Rₐ)—R₂, optionally substituted —CH₂-(3- to 6-membered heterocycloalkyl), optionally substituted —C(O)-(3- to 6-membered heterocycloalkyl), optionally substituted —C(O)—(C₃₋₆cycloalkyl), optionally substituted —CH₂—(C₃₋₆cycloalkyl), —CH₂—N(Rₐ)—R₂, —CH₂—O—R₂, or —CH₂—(CO)—N(Rₐ)—R₂;
(v) R₁ is an optionally substituted C₁₋₆alkyl, optionally substituted C₃₋₆cycloalkyl, optionally substituted C₁₋₆alkoxy, optionally substituted 3- to 8-membered heterocycloalkyl (e.g., 3- to 7-membered or 3- to 6-membered heterocycloalkyl), —C(O)—R₂, —C(O)O—R₂, —OC(O)—R₂, —C(O)N(Rₐ)—R₂, —N(Rₐ)C(O)—R₂, —N(Rₐ)—R₂, or —O—R₂;
(vi) Rₐ is H, optionally substituted C₁₋₆alkyl, or optionally substituted C₃₋₆cycloalkyl; and
(vii) R₂ is optionally substituted C₁₋₆alkyl, optionally substituted C₃₋₆cycloalkyl, optionally substituted C₁₋₆alkoxy, optionally substituted aryl, optionally substituted heteroaryl, or optionally substituted 3- to 7-membered heterocycloalkyl;
provided that
(a) R₁ is not optionally substituted piperazine when A is unsubstituted furan or thiophene, and B is optionally substituted phenyl;
(b) R₁ is not optionally substituted piperazine when A is unsubstituted furan and B is unsubstituted furan or thiophene;
(c) R₁ is not optionally substituted piperazine, morpholine or pyrrolidine, when A is unsubstituted pyridine and B is unsubstituted phenyl; and
(d) R₁ is not unsubstituted morpholine or pyrrolidine when A is 3-methyl-2-quinoxalinyl and B is unsubstituted 1-pyrrolidinyl or 3-fluoro-1-pyrrolidinyl.

2. The compound according to claim 1, wherein X is —CH— and Y is —N—.

3. The compound according to claim 1, wherein X is —N— and Y is —CH—.

4. The compound according to claim 1, wherein X is —N— and Y is —N—.

5. The compound according to claim 1, wherein A is an optionally substituted heteroaryl.

6. The compound according to claim 5, wherein said heteroaryl is selected from pyridine, pyrimidine, pyridazine, pyrazine, triazine, thiophene, furan, pyrrole, oxazole, imidazole, thiazole, pyrazole, isoxazole, isothiazole, triazole (e.g., 1,2,3-triazole, or 1,2,4-triazole), oxadiazole (e.g., 1,2,3-oxadiazole, or 1,2,4-oxadiazole), thiadiazole (e.g., 1,2,3-thiadiazole, or 1,2,4-thiadiazole), tetrazole (e.g., 1,2,3,4-tetrazole), and indole.

7. The compound according to claim 5, wherein said heteroaryl is selected from pyrrole, oxazole, imidazole, thiazole, pyrazole, isoxazole, isothiazole, indole, benzimidazole, benzoxazole, benzothiazole, indazole, benzisoxazole, and benzisothiazole.

8. The compound according to claim 5, wherein said heteroaryl is pyrazole (e.g., 3-substituted-1-pyrazolyl or 1-substituted-3-pyrazolyl).

9. The compound according to claim 1, wherein said heteroaryl is substituted with one or more groups selected from OH, CN, C₁₋₆alkyl (e.g., methyl), halogen (e.g., F), C₁₋₆alkoxy (e.g., methoxy), hydroxyC₁₋₆alkyl (e.g., 2-hydroxyethyl or 1-hydroxyethyl), C₁₋₆alkyl-C₁₋₆alkoxy (e.g., methoxymethyl or ethoxymethyl), C₁₋₆alkyl-C₁₋₆thioalkyl (e.g., methylthiomethyl), haloC₁₋₆alkyl (e.g., CHF₂ or CF₃), haloC₁₋₆alkoxy (e.g., OCF₃), carboxy (COOH), aryl, heteroaryl, C₃₋₆cycloalkyl, 3- to 10-membered heterocycloalkyl, and 3- to 10-membered heterocycloalkenyl, wherein said alkyl, alkoxy, thioalkyl, aryl, heteroaryl, cycloalkyl, heterocycloalkyl, and heterocycloalkenyl, are each optionally independently substituted with one or more groups selected from OH, CN, C₁₋₆alkyl (e.g., methyl or t-butyl), halogen (e.g., F or Br), C₁₋₆alkoxy (e.g., methoxy or ethoxy), haloC₁₋₆alkyl (e.g., CF₃), haloC₁₋₆alkoxy (e.g., OCF₃), carboxy (COOH), C₃₋₆cycloalkyl, and 5- or 6-membered heterocycloalkyl.

10. The compound according to claim 9, wherein said heteroaryl is substituted with aryl (e.g., phenyl) or heteroaryl (e.g., pyridyl or pyrimidinyl), wherein said aryl or heteroaryl is optionally substituted with one or more groups selected from OH, CN, C₁₋₆alkyl (e.g., methyl or t-butyl), halogen (e.g., F or Br), C₁₋₆alkoxy (e.g., methoxy or ethoxy), hydroxyC₁₋₆alkyl (e.g., 2-hydroxyethyl or 1-hydroxyethyl), C₁₋₆alkyl-C₁₋₆alkoxy (e.g., methoxymethyl or ethoxymethyl), C₁₋₆alkyl-C₁₋₆thioalkyl (e.g., methylthiomethyl), haloC₁₋₆alkoxy (e.g., OCF₃), and haloC₁₋₆alkyl (e.g., CHF₂ or CF₃).

11. The compound according to claim 9, wherein said heteroaryl is substituted with phenyl substituted with one, two or three groups independently selected from CN, C₁₋₆alkyl (e.g., methyl or t-butyl), halogen (e.g., F or Br), C₁₋₆alkoxy (e.g., methoxy or ethoxy), hydroxyC₁₋₆alkyl (e.g., 2-hydroxyethyl or 1-hydroxyethyl), C₁₋₆alkyl-C₁₋₆alkoxy (e.g., methoxymethyl or ethoxymethyl), haloC₁₋₆alkoxy (e.g., OCF₃), and haloC₁₋₆alkyl (e.g., CF₃).

12. The compound according to claim 1, wherein B is selected from optionally substituted pyridine and pyrimidine, optionally wherein B is unsubstituted pyridine, e.g., 2-pyridinyl, 3-pyridinyl, or 4-pyridinyl.

13. (canceled)

14. The compound according to claim 1, wherein B is selected from morpholine, piperidine, 1,2,3,6-tetrahydropyridinyl, piperazine, tetrahydropyran, pyrrolidine, tetrahydrofuran, oxetane, azetidine, oxirane, and aziridine, each optionally substituted (e.g., with C₁₋₆alkyl (e.g., N-substituted)).

15. The compound according to claim 1, wherein R₁ is C₁₋₆alkyl, C₃₋₆cycloalkyl, or C₁₋₆alkoxy, each substituted with an optionally substituted 3- to 8-membered heterocycloalkyl.

16. The compound according to claim 1, wherein R₁ is —C(O)—R₂, —C(O)O—R₂, —OC(O)—R₂, —C(O)N(Rₐ)—R₂, —N(Rₐ)C(O)—R₂, —N(Rₐ)—R₂, or —O—R₂; and wherein R₂ is an optionally substituted 3- to 8-membered heterocycloalkyl.

17. The compound according to claim 1, wherein R₁ is an optionally substituted 3- to 8-membered heterocycloalkyl.

18. The compound according to claim 15, wherein said 3- to 8-membered heterocycloalkyl is selected from aziridine, azetidine (e.g., azetidine-3-one or azetidine-3-spiro-oxetane), oxetane, pyrrolidine (e.g., 3,3-difluoropyrrolidin-1-yl), pyrrolidinone (e.g., 1-pyrrolidin-3-one), tetrahydrofuran, dihydropyran, tetrahydropyran, morpholine, piperidine (e.g., 4,4-difluoropiperidine), piperazine, oxazepane (e.g., 1,4-oxazepane), hexahydro-1H-furo[3,4-c]pyrrole, and oxa-azaspiro[3.3]heptane (e.g., 2-oxa-6-azaspiro[3.3]heptan-6-yl), each optionally substituted.

19. The compound according to claim 18, wherein said heterocycloalkyl is morpholine (e.g., 2-methyl-4-morpholinyl, or 3-methyl-4-morpholinyl, or 4-morpholinyl (i.e., N-morpholinyl)).

20. (canceled)

21. The compound according to claim 1, wherein the compound is selected from:

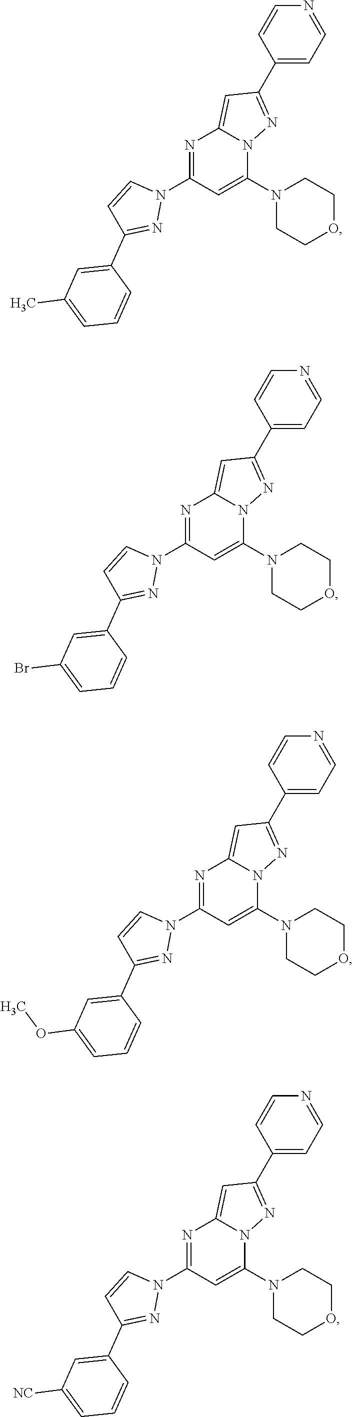 [SMILES: Cc1cccc(-c2ccn(-c3cc(N4CCOCC4)n4nc(-c5ccncc5)cc4n3)n2)c1 ; Brc1cccc(-c2ccn(-c3cc(N4CCOCC4)n4nc(-c5ccncc5)cc4n3)n2)c1 ; COc1cccc(-c2ccn(-c3cc(N4CCOCC4)n4nc(-c5ccncc5)cc4n3)n2)c1 ; N#Cc1cccc(-c2ccn(-c3cc(N4CCOCC4)n4nc(-c5ccncc5)cc4n3)n2)c1]

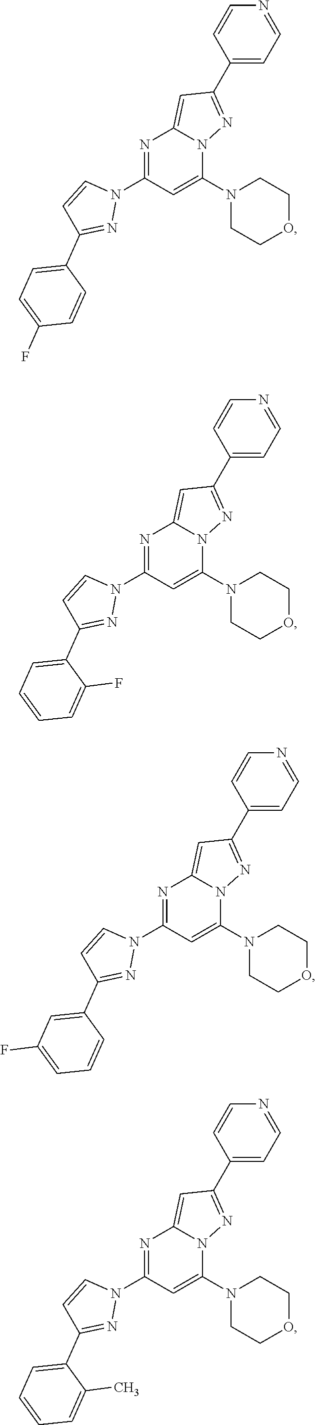 [SMILES: Fc1ccc(-c2ccn(-c3cc(N4CCOCC4)n4nc(-c5ccncc5)cc4n3)n2)cc1 ; Fc1ccccc1-c1ccn(-c2cc(N3CCOCC3)n3nc(-c4ccncc4)cc3n2)n1 ; Fc1cccc(-c2ccn(-c3cc(N4CCOCC4)n4nc(-c5ccncc5)cc4n3)n2)c1 ; Cc1ccccc1-c1ccn(-c2cc(N3CCOCC3)n3nc(-c4ccncc4)cc3n2)n1]

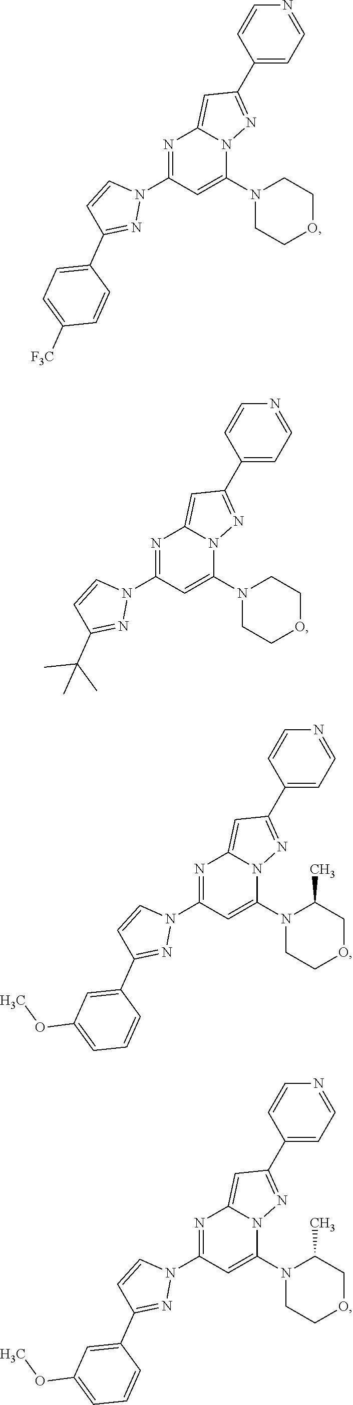 [SMILES: FC(F)(F)c1ccc(-c2ccn(-c3cc(N4CCOCC4)n4nc(-c5ccncc5)cc4n3)n2)cc1 ; CC(C)(C)c1ccn(-c2cc(N3CCOCC3)n3nc(-c4ccncc4)cc3n2)n1 ; COc1cccc(-c2ccn(-c3cc(N4CCOC[C@@H]4C)n4nc(-c5ccncc5)cc4n3)n2)c1 ; COc1cccc(-c2ccn(-c3cc(N4CCOC[C@H]4C)n4nc(-c5ccncc5)cc4n3)n2)c1]

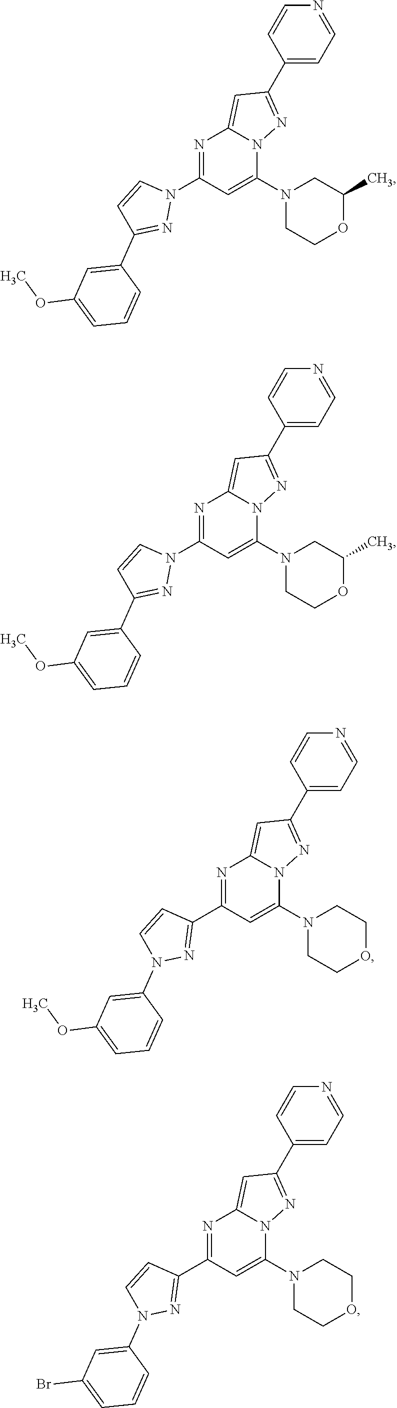 [SMILES: COc1cccc(-c2ccn(-c3cc(N4CCO[C@H](C)C4)n4nc(-c5ccncc5)cc4n3)n2)c1 ; COc1cccc(-c2ccn(-c3cc(N4CCO[C@@H](C)C4)n4nc(-c5ccncc5)cc4n3)n2)c1 ; COc1cccc(-n2ccc(-c3cc(N4CCOCC4)n4nc(-c5ccncc5)cc4n3)n2)c1 ; Brc1cccc(-n2ccc(-c3cc(N4CCOCC4)n4nc(-c5ccncc5)cc4n3)n2)c1]

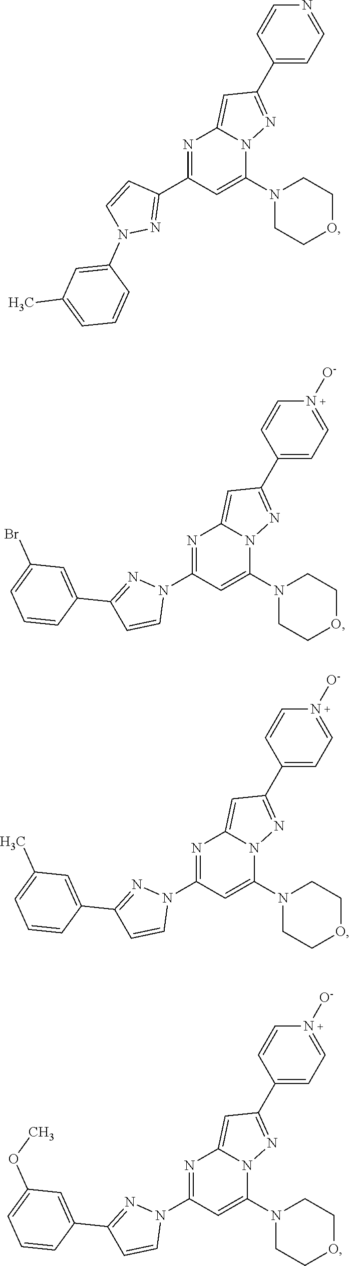

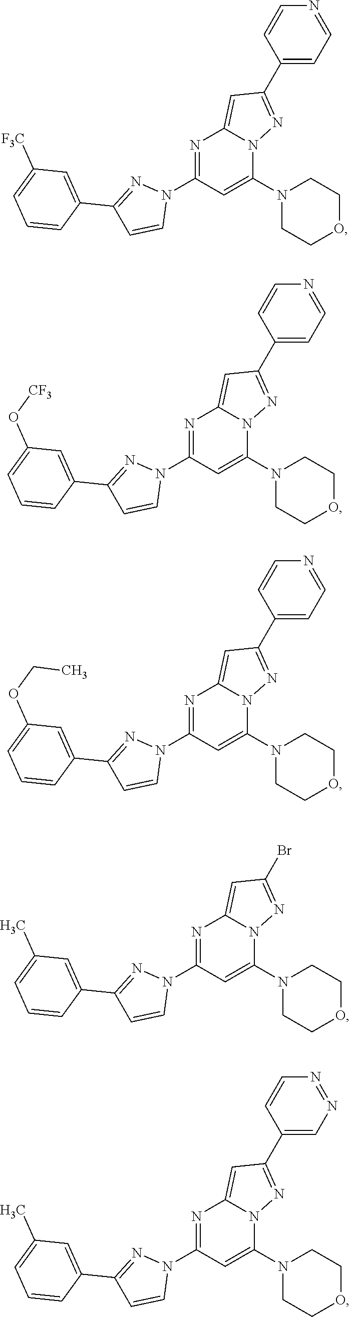

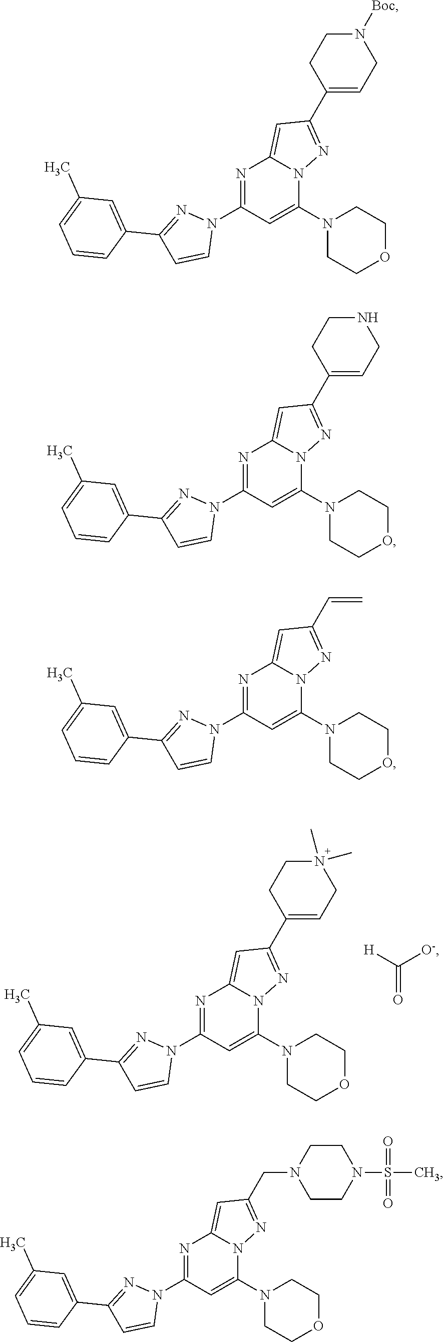

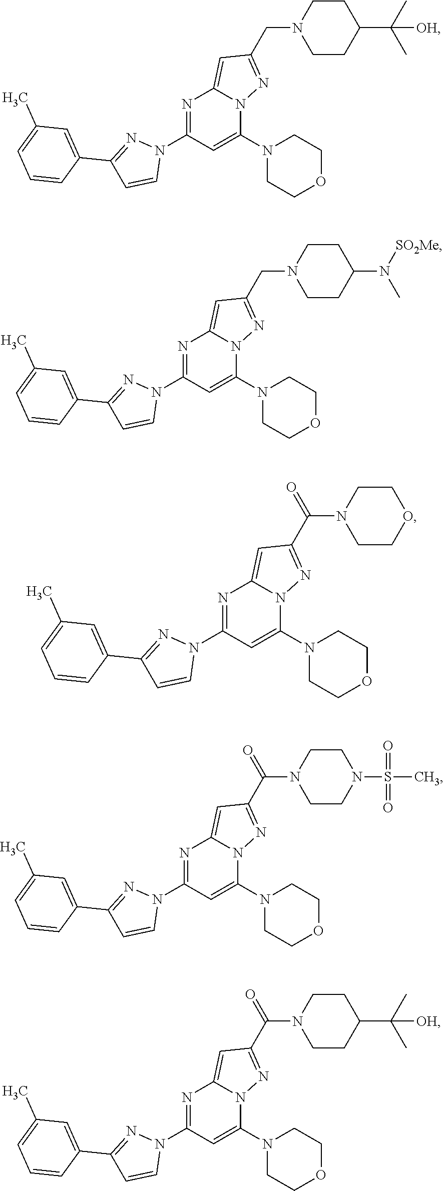 [SMILES: Cc1cccc(-c2ccn(-c3cc(N4CCOCC4)n4nc(CN5CCC(C(C)(C)O)CC5)cc4n3)n2)c1 ; Cc1cccc(-c2ccn(-c3cc(N4CCOCC4)n4nc(CN5CCC(N(C)S(C)(=O)=O)CC5)cc4n3)n2)c1 ; Cc1cccc(-c2ccn(-c3cc(N4CCOCC4)n4nc(C(=O)N5CCOCC5)cc4n3)n2)c1 ; Cc1cccc(-c2ccn(-c3cc(N4CCOCC4)n4nc(C(=O)N5CCN(S(C)(=O)=O)CC5)cc4n3)n2)c1 ; Cc1cccc(-c2ccn(-c3cc(N4CCOCC4)n4nc(C(=O)N5CCC(C(C)(C)O)CC5)cc4n3)n2)c1]

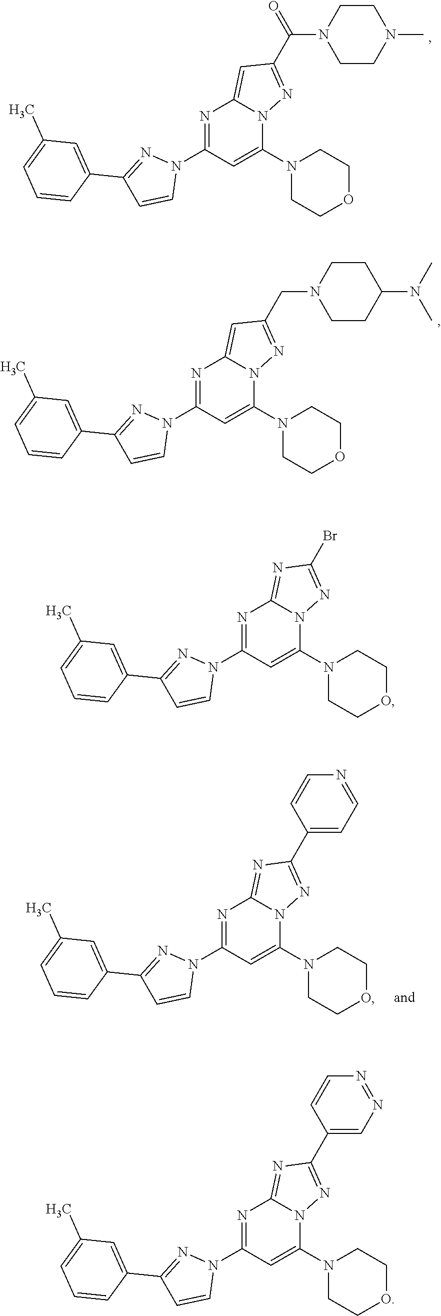 [SMILES: Cc1cccc(-c2ccn(-c3cc(N4CCOCC4)n4nc(C(=O)N5CCN(C)CC5)cc4n3)n2)c1 ; Cc1cccc(-c2ccn(-c3cc(N4CCOCC4)n4nc(CN5CCC(N(C)C)CC5)cc4n3)n2)c1 ; Cc1cccc(-c2ccn(-c3cc(N4CCOCC4)n4nc(Br)nc4n3)n2)c1 ; Cc1cccc(-c2ccn(-c3cc(N4CCOCC4)n4nc(-c5ccncc5)nc4n3)n2)c1 ; Cc1cccc(-c2ccn(-c3cc(N4CCOCC4)n4nc(-c5ccnnc5)nc4n3)n2)c1]

22. A pharmaceutical composition comprising the compound according to claim 1, in free or pharmaceutically acceptable salt form, in admixture with a pharmaceutically acceptable diluents or carrier.

23. A method for the treatment or prophylaxis of a disease or disorder characterized by dysregulation of phosphoinositide-mediated signal transduction pathways or which may be ameliorated by modulating (e.g., inhibiting) PIKFYVE-dependent signaling pathways or by modulating (e.g., inhibiting) endosome formation or trafficking, comprising administering to a patient in need thereof a therapeutically effective amount of the compound according to claim 1, in free or pharmaceutically acceptable salt form, optionally wherein the disease or disorder is a hyperproliferative disease (e.g., cancer, such as non-Hodgkin lymphoma, multiple myeloma, melanoma, liver cancer, glioblastoma, multiple myeloma, prostate cancer or breast cancer), an autoimmune disease (such as Crohn's disease or rheumatoid arthritis), a neurological disease (such as amyotrophic lateral sclerosis (ALS) and fronto-temporal dementia (FTD), and in particular C9FTD/ALS), diabetes or prediabetes, or Francois-Neetens corneal fleck dystrophy, or an infection by an enveloped virus, e.g., Ebola, influenza A, vesicular stomatitis virus, Lassa fever virus, lymphocytic choriomeningitis virus, or a coronavirus (including MERS-CoV, SARS-CoV and SARS-CoV-2).

24. (canceled)

25. (canceled)

26. (canceled)

27. (canceled)

28. (canceled)

29. (canceled)
